SEQUENCE LISTING (327 sequences)

SEQ ID NO: 1 - protein, 233 aa, Homo sapiens
EVQLLESGGGLVQPGGSLRLSCAASGFTFSSYAMSWVRQAPGKGLEWVSTISTGGGYTYFPDSVKGRFTISRDNSKNTLYLQMNSLRAEDTAVYYCARQGDFGDWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 2 - protein, 110 aa, Homo sapiens
EVQLVESGGALIQPGGSLRLSCAASGFTVDSNYIMWVRQAPGKGLEWVSGIYSDGRTYYADSVKGRFTISRENSKNTVYLQMNTLKAEDTALYYCASVAAEVDFWGQGTL

SEQ ID NO: 3 - protein, 126 aa, Homo sapiens
QITLKESGPTLVKPSQTLTLTCSVSGSSLSTSGVGVGWLCQPPGKALEWLALIYWDDDSRYNPSLEGRLTITKDTSKHQVILTMTDMDPVDTATYYCAHRHYYDFWSGYYDWFDPWGQGIPVTVSS

SEQ ID NO: 4 - protein, 123 aa, Homo sapiens
QVQLVESGGGLIQPGGSLRLSCAASGVTFSTYEVNWVRQAPGKGLEWVSYISNSGDTIYYSDSVKGRFSISRDNAKTSLYLQMNSLRVEDTAVYYCATGYYYDRGGNYNGDYWGQGTLVTVSS

SEQ ID NO: 5 - protein, 114 aa, Homo sapiens
QVQLVQSGAEMKNPGASVKVSCKASGYTFTGYYMHWVRQAPGQGLEWMGLVNPNTGDTTYAQKFQGRVTMTRDTSITTAYMELSRLRSDDTAVYYCARLGLDYWGQGTLVTVSS

SEQ ID NO: 6 - protein, 122 aa, Homo sapiens
EVQLLESGGGLVQPGGSLRLSCAASGFTFRNTGMSWVRQAPGKGLEWVSAISGSGGITNLADSVKGRFSISRDNSKSTLYLQMNSLRAEDTAIYYCAKDSTYYYDSNGYTDWGQGTLVTVSS

SEQ ID NO: 7 - protein, 116 aa, Homo sapiens
EVQLVESGGGLVQPGGSLRLSCSASGFTFSGYAMYWVRQAPGKGLEYVSGITSNGGGTYYADSVKGRFTISRDNSKNTLYLQTSSLRAEDTAIYYCVRDNWGFDYWGQGTLVTVSS

SEQ ID NO: 8 - protein, 120 aa, Homo sapiens
QEQLVQSGAEVKKPGASVKVSCKASGYSFIVYYIHWLRQAPGQGLEWMGWINPRTGDTNYAQTFQGRVTMTRDTSISTAYMDLGRLRFDDTAVYYCARDVELRYGTGWFDPWGQGTLVTV

SEQ ID NO: 9 - protein, 123 aa, Homo sapiens
QVQLVESGGGLIQPGGSLRLSCAASGVTFSTYEMNWVRQAPGKGLEWVSYISNSGDTIYYSDSVKGRFSISRDNAKTSLYLQMNSLRVEDTAVYYCATGYYYDRGGNYNGDYWGQGTLVTVSS

SEQ ID NO: 10 - protein, 121 aa, Homo sapiens
QVQLQESGPGLVKPSETLSLTCSVSGGSISTYYWSWIRQPPGKGLEWIGYIYNSGYAYYNPSLKSRVTISLDTSENQFSLKLSSVTAPDTAVYYCAREAVAGTGGWNFDLWGRGTLVTVSS

SEQ ID NO: 11 - protein, 123 aa, Homo sapiens
EVQLLESGVEVKKPGESLRISCKGSGYSFTSYWIGWVRQMPGKGLEWMGIIYPGDSETRYSPSFQGQVAISADKSISTAYLRWSSLKASDTAIYYCASMGYSIVVAGTYFDYWGQGTLVTVSS

SEQ ID NO: 12 - protein, 125 aa, Homo sapiens
QVQLVQSGAEVKKLGASVKVSCKASGYTFTDYYIHWVRQAPGQGLEWMGWINPNSGGTNYAQKFQGRVTVTRDTSINTVYMELSRLRSDDTAVYYCARDHCSGGSCPALGAIDYWGQGTLVTVSS

SEQ ID NO: 13 - protein, 123 aa, Homo sapiens
KVQLVQSGVEVKKPGESLRISCKGSGYSFTSYWIGWVRQMPGKGLEWVGIIYPADSDSRYSPSFQGQVTISADKSITTAYLQWRSLKASDTAMYYCASRSGDYGINDGTFDIWGQGTMVTVSS

SEQ ID NO: 14 - protein, 118 aa, Homo sapiens
QVQLQESGPGLVKPSETLSLICTVSGYSISSGYYWGWIRQPPGKGLEWIGNIYHNENTHYNPSLKSRLTMSVDTSKNQFSLNLSSVPAADTAVYYCARRMRAVAFDYWGQGTLVTVSS

SEQ ID NO: 15 - protein, 121 aa, Homo sapiens
EVQLVESGAEVKKPGSSVKVSCKASGGPFSNYAISWVPQAPGQGLEWMGGIIPIFSTANYAQKFQGRVTITADESTSTAYMELTGLKSDDTAVYYCARGGLKLDFYYMDVWGEGSTVTVSS

SEQ ID NO: 16 - protein, 119 aa, Homo sapiens
EVQLGESGGGLVKPGESLRLSCAASGFTFSNIWMSWVRQAPGKGLEWVGRIKTKTEGETTDYAAPVKGRFTISREDSINTLFLQMNSLEIEDTAVYYCTTDRPLSANGDAFDIWGQGTM

SEQ ID NO: 17 - protein, 127 aa, Homo sapiens
QVQLQESGGGLVQPGGSLRLSCGASGFTFSSYAMSWVRQAPGKGLEWVSAISGSGGSTYYADSVKGRFTISRDNSKNTLYLQMNSLRAEDTAVYYCAKDDSHLVVVPRPTRDAFDIWGQGTMVTVSS

SEQ ID NO: 18 - protein, 124 aa, Homo sapiens
EEHLLESGGGLVQPGGSLRLSCAPSGFSFSSYAMSWVRQAPGKGLEWVSGISSSGGGTFYADSVKGRFAISRDNSNNALYLQMGSLRADDTAVYYCARASYYYHSGYYYYMDVWGKGTTVTVSS

SEQ ID NO: 19 - protein, 119 aa, Homo sapiens
QVQLQEPGGGLVQPGGSLRLSCAASGFTFSSYAMSWVRQAPGKGLEWVSAISGSGGSTYYADSVKGRFTISRDNSKNTLYLQMNSLRAEDTAVYYCAKDQGIAAAPNYWGQGTLVTVSS

SEQ ID NO: 20 - protein, 115 aa, Homo sapiens
EVQLLESGGGLVQPGGSLRLSCAASGFTFSNYAMRWVRQAPGGGLEWVSAISAGSATTYYADSVKGRFTISRDNSKNTLYLEMNSLRAEDTAMYYCARRDPGQGHGMDVWGQGTT

SEQ ID NO: 21 - protein, 125 aa, Homo sapiens
QVQLVESGGGVVQPGRSLRLSCAASGFTFSSLYMHWVRQAPGKGLEWLAVISFDASQYYAAYSVKGRFTISRDNSKNVLSLHMDSLRVEDTAVYYCARGESPSFGVVVSLGMDVWGQGTMVTVSS

SEQ ID NO: 22 - protein, 117 aa, Homo sapiens
QVQLQQSGPGLVKPSQTLSLTCAISGDSISSNSVAWNWIRQSPSRGLEWLGRTYYRSKWFNDYAVSVKSRITINPDTSKNQFSLHLNSVTPEDTGVYFCAREDRAIFHSGMDVWGQG

SEQ ID NO: 23 - protein, 114 aa, Homo sapiens
QVQLQQSGPGLMKPSQTLSLTCAISGDNVSSNSAAWNWIRQSPSRGLEWLGRTYYTSKWYNNYAISVKSRITINPDTSKNQFSLQLNSVTPEDTAVYYCARDRRNGFIDYWGQG

SEQ ID NO: 24 - protein, 121 aa, Homo sapiens
QVQLVQSGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGGGWNDVFDYWGQGTLVTVSS

SEQ ID NO: 25 - protein, 120 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLASIAARFDYWGQGTLVTVSS

SEQ ID NO: 26 - protein, 129 aa, Homo sapiens
QVQLQESGPGLVKPSQTLSLTCTVSGGSISSGSYSWSWIRQPAGKGLEWIGVFHTSGSTNYHPSLKSRITMSVDTSKNQFSLKLSSVTAADTAVYYCTRRGDYESTVGQHNYYYYLDVWGQGTTVTVSS

SEQ ID NO: 27 - protein, 121 aa, Homo sapiens
QVQLMESGGGVVQPGRSLRLSCAASGFAFSSFGMDWVRQSPGNGLEWVAGISYHGSNKYYADSVKGRFTISRDNSKNTLYLQMNSLRAEDTAVYYCAKDPNKLEPGYFDSWGKGTLVTVSS

SEQ ID NO: 28 - protein, 118 aa, Homo sapiens
QVQLQQSGPGLVKPSQTLSLTCAISGDSVSSNSAAWNWIRQSPSRGLEWLGRTYYRSKWYNDYAVSVESRITINPDTSKNQFSLQLNSVTPEDTAVYYCARDRVSGWYRGAFDIWGQG

SEQ ID NO: 29 - protein, 117 aa, Homo sapiens
QVHLVQSGAEVKKPGASVKVSCRVPGYTLTDFSFHWVRQPPGKALEWMGGFDPEKAETVYAQKFQGRVTMTEDTSTDTAYMEPVSLRSDDTAVYYCATDLGWEARLWGQGTQVTVSS

SEQ ID NO: 30 - protein, 127 aa, Homo sapiens
EVQLVESGGGLVQPGGSLRLSCAASGFIFRNYWMHWVRQGPGKGLEWVSRIKNDGTDTTYADSVKGRFTISRDNAKNTLHLQMTSLRVEDTALYYCVIIPGYCSTTSCETFYYMDAWGKGTTVIVSS

SEQ ID NO: 31 - protein, 122 aa, Homo sapiens
EVQLVESGGGLVQPGGSLRLSCAASGFTFSDYAMSWVRQTPEKGLEWVSAIGGNDGRTYYTDSVKGRFTISRDNSKNTLYLHMSSLRVEDTAVYYCAKPGQPYFYGCPSEHWGQGTLVTVSS

SEQ ID NO: 32 - protein, 123 aa, Homo sapiens
QVQLQESGGGSVQPGRSLRLSCTASGFTFGDYAMSWVRQAPGKGLEWVGFIRSKPYGGTTEYAASVKGRFTISRDDSKSIAYLQMNSLKTEDTAVYYCTYGRSGGDYLLLRYWGQGTLVTVSS

SEQ ID NO: 33 - protein, 115 aa, Homo sapiens
QVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARVRRRYSSASKIIFGSGT

SEQ ID NO: 34 - protein, 118 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGYSYDILTYYMDVWGQG

SEQ ID NO: 35 - protein, 122 aa, Homo sapiens
EVQLLESGGGVVQPGRSLRLSCAASGFTFSSYAMHWVRQAPGKGLEWVAVISYDGSNKYYADSVKGRFTISRDNSKNTLYLQMNSLRAEDTAVYYCARDLTLRFLEWLLDYWGQGTLVTVSS

SEQ ID NO: 36 - protein, 76 aa, Homo sapiens
QVQLQESGGGLVKPGGSLRLSCGASGFTFSNAWMSWVRQAPGKGLEWVGRIKSKTDGGTTDYAAPVKGRFTISRDD

SEQ ID NO: 37 - protein, 122 aa, Homo sapiens
QVQLVQSGAEVTRPGSSVKVSCQASGGALTNYAFAWLRQAPGQGLAWVGGILPVYGTPDYADNLKDRVTITSDASTNTAYLELTSLRSEDTAVYYCARDRNSSWYSGAFDVWGQGTMVTVSS

SEQ ID NO: 38 - protein, 122 aa, Homo sapiens
GVQLVESGGGLVKPGGSLRLSCAASGFTFSSYSMNWVRQAPGEGLEWVSSISSSSSYIYYADSVKGRFTISRDNAKNSLYLQMNSLRAEDTAVYYCARCARVGIVGAKGPPWGQGTLVTVSS

SEQ ID NO: 39 - protein, 119 aa, Homo sapiens
EVQLVETGGGVVQPGRSLRPSCAASGFIFSDSAIHWVRQASGKGMEWVSSISSSSSYIYYADSVKGRFTISRDNAKNSLYLQMNSLRAEDTAVYYCAQEGIVGDGMDVWGRGTTVTVSS

SEQ ID NO: 40 - protein, 123 aa, Homo sapiens
QVQLQESGPGLVKPSQTLSLTCTVYGGSFSGYYWSWIRQPPGKGLEWIGEINHSGSTNCNPSLKSRVTISVDTSKNQFSLKLSSVTAADTAVYYCARGGYKWLAQSLRYPPLWGQGTLVTVSS

SEQ ID NO: 41 - protein, 123 aa, Homo sapiens
QVQLVQSGAEVRKPGSSVKVSCKASGGTFSTYAFSWVRHSPGQGLEWMGGITPMFGTARYAQNFQGRVTITADESTRTAYMELSSLRSEDTAVYYCARGWLDPSMATTPFGFWGQGTVVTVSS

SEQ ID NO: 42 - protein, 103 aa, Homo sapiens
QVQLQQSGPGLVKPSQTLSLTCAISGDSVSSNSASWNWIRQSPSRGLEWLGRTYYRSKWYNDYAVSVKSRVTINPDTSKNQFSLQLNSVTPEDTAVYYCARDL

SEQ ID NO: 43 - protein, 120 aa, Homo sapiens
QVQLVQSGPEVKKPGTSVKVSCKASGFTFTSSAVQWVRQARGQRLEWMGWIVVGNGNTNYAQKFQERVTITRDMSTSTAYMELSSLRSEDTAVYYCAADPHYGGDGFDIWGQGTMVTVSS

SEQ ID NO: 44 - protein, 123 aa, Homo sapiens
QVQLVQSGAEVKKPGSSVKVSCKASGGTFDRYTINWVRHSPGQGLEWMGGIIPMFGTPNYAEKFRGRVAISAEKSTTTAYMEMSSLTSEDTAIYYCARGSGEHTNMVVPFDYWGQGTLVTVSS

SEQ ID NO: 45 - protein, 124 aa, Homo sapiens
QVQLVQSGGGLVQPGRSLRLSCTASGFTFGDYAMSWVRQAPGKGLEWVGFIRSKAYGGTTEYAASVKGRFTISRDDSKTIAYLQMNSLKTEDTAVYYCTRDYSSTVTTPPFDYWGQGTLVTVSS

SEQ ID NO: 46 - protein, 123 aa, Homo sapiens
QVQLVESGGGLIQPGGSLRLSCAASGVTFSTYEMNWVRQAPGKGLEWVSYISNSGDTIYYSDSVKGRFGISRDNAKTSLYLQMNSLRVEDTAVYYCATGYYYDRGGNYNGDYWGQGTLVTVSS

SEQ ID NO: 47 - protein, 99 aa, Homo sapiens
EVQLLESGGGLVQPGGSLRLSCAASGFTFNNYAMSWVRQAPGKGLEWVSSISGRGGSTCYADSVKGRFTISRDNYKNTLYLQMNSLRAEDTAVYYCAKY

SEQ ID NO: 48 - protein, 117 aa, Homo sapiens
EMQLVESGGAFVQPGGSLKLSCAASGFNFSDSTIHWVRQASGKSLEWVGHIENKTKNYATIYRASVKGRFTISRDDSKNMAFLQMDSLRPDDTALYYCTPPPEVESLRSWGRGTLVT

SEQ ID NO: 49 - protein, 120 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPDVVVPAADYWGQGTLVTVSS

SEQ ID NO: 50 - protein, 53 aa, Homo sapiens
QVQLQESGGGLVKPGGSLRLSCADLGFTFSSYSMNWVRQAPGKGLEWVSSIST

SEQ ID NO: 51 - protein, 124 aa, Homo sapiens
QVQLQESGPGLVKPSQTLSLTCTVSGGSISSSYFWSWIRQPAGKGLEWIGRIYYSGSTNYNPSLKNRVTMAVDASNNQFSLRLSSVTAADTAVYYCARLAAPIANNWGSTQYWGLAGTTVTVSS

SEQ ID NO: 52 - protein, 127 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREVVGCSSTSCYYYYGMDVWGQGTTVTVSS

SEQ ID NO: 53 - protein, 120 aa, Homo sapiens
QVKLLESGPGLVKPSQTLSLTCAISGDSVSSNSAAWDWIRQSPSRGLEWLGRTYYRSKWYNDYAVSVKSRITINPDTSKNQFSLQLNSVTPEDTGGYYCTKDSGSDFDVWGQGTLVTVSS

SEQ ID NO: 54 - protein, 115 aa, Homo sapiens
QLVQSGAEVKKPGSSVKVSCKASGGTFSSYAISWVRQAPGQGLEWMGGIIPIFGTANYAQKFQGRVTITADESTSTAYMELSSLRSEDTAVYYCARQAHSISILSVYVTTFDYWA

SEQ ID NO: 55 - protein, 130 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARVHVPTGILGGHMVQGVIHYYYGMDVWGQG

SEQ ID NO: 56 - protein, 120 aa, Homo sapiens
QVQLQESGAEVKKSGASVKVSCKASGYTFTSYYMHWVRQAPGQGLEWMGIINPSGGSTSYAQKFQGRVTMTRDTSTSTVYMELSSLRSEDTAVYYCARDSGYDGHGMDVWGRGTLVTVSS

SEQ ID NO: 57 - protein, 118 aa, Homo sapiens
QVQLVQSGGGLVQPGGSLRLSCAASGFTFSTYSMSWVRQAPGKGLEWVANIKQDGNERYYVDSVKGRFTISRDNTKNSLFLQINSLRAEDTAVYYCAREGWISALNGWGQGTLVTVSS

SEQ ID NO: 58 - protein, 121 aa, Homo sapiens
EVQLVESGGGLVQPGGSLRLSCAASGFTFTNYAMSWVRRAPGKGLEWVSTISGSGSSTFYADSVKGRFTISRDDSRNTLYLQLNSLRAEDTAVYYCAKLKSGTTFGAFDIWGQGTMVSVSS

SEQ ID NO: 59 - protein, 123 aa, Homo sapiens
QVRLVESGGGLIQTGGSLRLSCAASGVTFSTYEMSWVRQAPGKGLEWVSYISKGGDTIYYSDSVKGRFIISRDNAKTSLYLQMNSLRVEDTAVYYCATGYYYDSGGNYNGDYWGQGTLVTVSS

SEQ ID NO: 60 - protein, 126 aa, Homo sapiens
QITLKESGPTLVNPTQTLTLTCIVSGSSLSTRGVGVGWLRQPPGKALEWLALIYWDGDARYSPSLEGRLTITKDTSKNQVVLTMTNMEPVDTATYYCAHRHYYDFWSGYYDWFDPWGQGTLVTVSS

SEQ ID NO: 61 - protein, 122 aa, Homo sapiens
EVQLVESGGGFVQPGGSLRLSCAASGFTFSTYWMSWVRQAPGKGLEWVANINQDGSEKYYVDSVKGRFTISRDNAKNSLYLQMNSLRAEDTAIYYCARDQNSVRYYGDQKRINFDYWGQGTL

SEQ ID NO: 62 - protein, 105 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLVVGMV

SEQ ID NO: 63 - protein, 134 aa, Homo sapiens
LVQLQESGPGLVKPSQTLSLTCTVSGASLIRGDYYWSWIRQSPGKGLEWIGYIYSSGTTFYNPSLRSRPAISIDTSKNQFSLKLSSVTAADTAVYYCARANIEVVPAIAINEKHSFYFYSMDVWGQGTTVTVSS

SEQ ID NO: 64 - protein, 120 aa, Homo sapiens
QVQLQQSGPGLVKPSQTLSLTCAISGDSVSSNSAAWNWIRQSPSRGLEWLGRTYYRSKWYNDYAVSVKSRITINPDASKNQFSLQLNSVTPEDTPVYYCARALGYSSGWYANYFDYWGQG

SEQ ID NO: 65 - protein, 126 aa, Homo sapiens
QVQLVQSGGGVVQPGRSLRLSCAASGFTFSNSGMHWVRQAPGKGLEWVAIISYDGSKKYYADSVKGRFTISRDDSKNTLFLQMNSLRLEDTAVYYCAKDGVGIVEIPAAFDWFDPWGQGTLVTVSS

SEQ ID NO: 66 - protein, 114 aa, Homo sapiens
QVQLQQSGPGLVKPSQTLSLTCAISGDTVSSDSAAWDWIRQSPSRGLEWLGRTYYRSKWFNDYALSVKSRLTITPDTSKNQFSLQLNSLTPEDTAMYYCTREFLPSDAFDIWGQ

SEQ ID NO: 67 - protein, 117 aa, Homo sapiens
ELQLVESGGDLVQPGRSLRLSCTGSGFNFGDYAMSWVRQAPGKGLEWVGSIKDEGNGGTTEYAASVKGRFIISRDDSKSVAYLQMNSLTTADTAVYYCTFFGIYDYWGQGTLVTVSS

SEQ ID NO: 68 - protein, 121 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKENYGSGSYSFDPWGQGTLVTVSS

SEQ ID NO: 69 - protein, 126 aa, Homo sapiens
QITLKESGPTLVNPTQTLTLTCIVSGSSLSARGVGVGWLRQPPGKALEWLALIYWDGDRRYSPSLEGRLTITKDTSKNQVVLTMTNMEPVDTATYYCAHRHYHDFWFGYYDWFDPWGPGIPVTVSS

SEQ ID NO: 70 - protein, 126 aa, Homo sapiens
QITLKESGPTLVKPTQTLTLTCTVTGSSLSTSGVGVGWLRQPPGKALEWLALIYWDDDRRYSPSLEGRLTITPDTSKNQVVLTMTNMDPVDTATYYCAHRHYFDFWNGYYDWFDPWGQGTPVTVSS

SEQ ID NO: 71 - protein, 128 aa, Homo sapiens
QTQLVQSGAEVRKPGASVRVSCKASGYTFIDSYIHWIRQAPGHGLEWVGWINPNSGGTNYAPRFQGRVTMTRDASFSTAYMDLRSLRSDDSAVFYCAKSDPFWSDYYNFDYSYTLDVWGQGTTVTVSS

SEQ ID NO: 72 - protein, 121 aa, Homo sapiens
EVQLLESGGDLVQPGGSLRLSCAASGFTFSNYVMSWVRQAPGKGLEWVSSISGSDGGTYYTASVKGRFTISRENSKNTLYLQMNSLRAEDTAVYYCAQNMGDIRLTPLAYWGQGTLVTVSS

SEQ ID NO: 73 - protein, 121 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDEQIRDLVVYASWFDPWGQG

SEQ ID NO: 74 - protein, 121 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDISWGDLEGLDYWGQGTLVTVSS

SEQ ID NO: 75 - protein, 124 aa, Homo sapiens
QVQLQESGPGLVKPSQTLSLTCTVSGGSISSSYYWSWIRQPAGKGLEWIGRIYTSGSTNYNPSLKSRVTMAVDASNNQFSLRLSSVTAADTAVYYCARLAAPIANNWGSTQYWGLAGTTVTVSS

SEQ ID NO: 76 - protein, 125 aa, Homo sapiens
QVQLVQSGGGVVQPGRSLRLSCAASGFTFSSYAMHWVRQAPGKGLEWVAVISYDGSNKYYAESVKGRFTISRDNSKNTLYLRMNSLRTEETAVYYCARDGDVGSYGPGTYYPIDYWGQGTLVSVS

SEQ ID NO: 77 - protein, 124 aa, Homo sapiens
EVQLVESGGGLVEPGGSLRLSCAGAGFTFSRYGLNWVRPAPGKGLEWVSSISTSSSYIYYAASVKGRFTISRDNARNSLSLQMNSLRAEDTAVYYCARSTRDGYSVNGEAFDMWGQGTMVTVSS

SEQ ID NO: 78 - protein, 125 aa, Homo sapiens
QVQLQQSGPGLVKLRGSVSLTCAVSGGAITSSNWWSWVRLSPGKGLEWIGEINHSGSTNHNPSLKSRVTISVDKSKNQFSLKLSSVTAADTAVYYCVSSRHGSGTTRYYYYMDVWGKGTTVTVSS

SEQ ID NO: 79 - protein, 127 aa, Homo sapiens
QVQLQESGPGLVKPSQTLSLTCTVSGGSINSGSYNWSWNRQPAGKGLEWSGGIYTSGSTNYPPSLKSRVTISVDTSKNQFSLKLSSVTGADTAVYYCARLASPAPIANNWGSTKNWGLAGTTVTVSS

SEQ ID NO: 80 - protein, 123 aa, Homo sapiens
QVQLVESGGGLIQPGGSLRLSCAASGVTFSTYEMNWVRQAPGKGLEWVSYISNSGDTIYYSDSVKGRFSISRDNAQTSLYLQMNSLRVEDTAVYYCATGYYYDRGGNYNGDYWGQGTLVTVSS

SEQ ID NO: 81 - protein, 126 aa, Homo sapiens
QVQLQESGPGLVKPSQTLSLTCTVSGGSISSSSSWSWIRQPPGKGLEWIGRLYYSGSTSYNPSLQSRVTMAVDTSKNQFSLRLSSVTAADTAVYYCARGVTPYPMIADWGSTYYWGLAGTTVTVSS

SEQ ID NO: 82 - protein, 126 aa, Homo sapiens
QITLKESGPTLVKPTQTLTLTCTVSGSSLSTSGVGVGWLRQPPGKALEWLALIYWDDDSRYSPSLEGRLTITKDTSKNQVVLTMTDMDPVDTGTYYCAHRHYFDFWNGYYDWFDPWGQGTLVTVSS

SEQ ID NO: 83 - protein, 127 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARRKDSSGWYSGYPDAFDIWGQGTMVTVSS

SEQ ID NO: 84 - protein, 117 aa, Homo sapiens
EMQLVQSGGAFVQPGGSLKLSCAASGFNFSDSTIHWVRQASGKSLEWVGHIEKKSKKYATIYRASVKGRFTISRDDSNNTAFLQMDSLRPDDTALYYCTPPPEVESLRSWGRGTLVT

SEQ ID NO: 85 - protein, 117 aa, Homo sapiens
QVQLVQSGGGLLQPGGSLRLSCAASGFTFSSSWMHWVRQPPGKGLVWVSRINPDGSSTTYADSVKGRFTISRDNAKNTLYLQMNSLSAEATAVYYCARPLSGNGDCWGQGTLVTVSS

SEQ ID NO: 86 - protein, 125 aa, Homo sapiens
EVQLLESGGGLVQPGGSLRLSCAASTASGFTFSTYAMSWVRQAPGKGLEWVSIISGGGGSTYYADSVKGRFTISRDNSKKNTLYLQMNSLRVEDTAVYYCAKVGYDYVWGTYRYTRFDYWGQGTL

SEQ ID NO: 87 - protein, 122 aa, Homo sapiens
QVQLQESGPGLVKPSGTLSLTCAVSGGSIITNTWWSWVRQSPGKGLEWIGEIYHGGNTNYNPSLKSRVTISVDKSTNQFSLKVSSVTAADTAVYYCARGRDTSSWFYYFDFWGQGTLVTVSS

SEQ ID NO: 88 - protein, 121 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAAWIQLSYGMDVWGKGTTVTVSS

SEQ ID NO: 89 - protein, 126 aa, Homo sapiens
QVQLQQWGAGLVKPSETLSLTCAVFGGSFSGYYWSWIRQPPGRGLEWIGEINHSGSTNYKTSLKSRVTISLDTSKNLFSLKLSSVTAADTAVYYCARGLLRGGWNDVDYYYGMDVWGQGTTVTVSS

SEQ ID NO: 90 - protein, 123 aa, Homo sapiens
QVTLKEFEGGVVQPGRSLRLSCAASGSNFSSYGMHWVRQAPGKGLEWVTVISYDGSDKYYADSVKGQFIASRDNSKNSLYLQMNSLRAEDTAVYYCARDGTTRTTATDYMDVWGKGTTVTVSS

SEQ ID NO: 91 - protein, 126 aa, Homo sapiens
QITLKESGPTLVKPTQTLTLTCTVTGSSLSTSGVGVGWLREPPGKALEWLALIYWDDDGRYSPSLEGRLTITKDTSKNQVVLTMTNMDPVDTATYYCAHRHYFDFWNGYYDWFDPWGQGTPVTVSS

SEQ ID NO: 92 - protein, 123 aa, Homo sapiens
QVQLVGSGGGLIQPGGSLRLSCAASGVTFSTYEMNWVRQAPGKGLEWVSYISNSGDTIYYSDSVEGRFSISRDNAKTSLYLQMNSLRVEDTAVYYCATGYYYDRGGNYNGDYWGQGTLVTVSS

SEQ ID NO: 93 - protein, 122 aa, Homo sapiens
EEHLLESGGGLVQPGGSLRLSCAPSGFSFSSYAMSWVRQAPGKGLEWVSGISSSGGGTFYADSVKGRFTISRDNSNNALYLQMDSLRADDTAVYYCARASYYYHSGYYYYMDVWGKGTTVTV

SEQ ID NO: 94 - protein, 114 aa, Homo sapiens
QVQLQQSGPGLVEPSQTLSLTCAISGDSVSSNGAAWNWLRQSPSRGLEWLGRTYSRSKWYKEYAVSVQSRITINSDTSKNQFFLQLNSVTPEDTAVYFCTRGFLRSGMDVWGQG

SEQ ID NO: 95 - protein, 99 aa, Homo sapiens
EVQLVQSGAEVKKPGESLKISCKGSGYSFTSYWIDWVRQMPGKGLEWMGIIYPGDYDTRYSPSFQGQVTFSADKSITTAYLQWSSLKASDTAMYYCARG

SEQ ID NO: 96 - protein, 125 aa, Homo sapiens
EVHLVESGGGLVQPGGSLKLSCAASGFTFSGAAMHWVRQASGKGLEWVGHIRDKANNYATTYAASVKGRFSISRDDSKNTAYLQMNSLRIEDTAVYFCTRNFTSLDSTGNSFGPWGRGTLVTVSS

SEQ ID NO: 97 - protein, 120 aa, Homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKDDSSGSYYFDYWGQGTLVTVSS

SEQ ID NO: 98 - protein, 121 aa, Homo sapiens
KVQLVHSGAEVKEPGESLKISCQVSGYSFTNYWIGWVRQIPGKGLEWMGIIYPVDSDTIYSPSFQGQVTISADKSITTAYLQWSSLKASDTAMYYCARRRDREGYRLFDYWGQGTLVTVSS

SEQ ID NO: 99 - protein, 115 aa, Homo sapiens
QVQLQQSGPGLVKPSQTLSLTCAISGNTVSSDSAAWDWIRQSPSRGLEWLGRTYYRSKWFNDYALSVKSRLTITPDTSKNQFSLQLNSLTPEDTAMYYCTREFLPSDAFDIWGQG

SEQ ID NO: 100 - protein, 133 aa, Homo sapiens
QVQLVQSGAEVKKPGSSVKVSCKASGGTFSRYAISWVRQAPGKGLEWVGGIVPVFGTTNYAQRFQGRVTITADESTSTAYMELSSLRSEDTAVYYCAKTGILGPYSSGWYPNSDYYYYGMDVWGQGTTVTVSS

SEQ ID NO: 101 - protein, 117 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARVGSSGSVNWFDPWGQG

SEQ ID NO: 102 - protein, 123 aa, Homo sapiens
QVQLVQSGADVKKPGESLKISCKGSGYNFSTNWIGWVRQMPGKGLEWVGIIYPADSDSRYSPSFQGQVTISADKSITTAYLQWRSLKASDTAMYYCASRSGDYGINDGTFDIWGQGTMVTVSS

SEQ ID NO: 103 - protein, 119 aa, Homo sapiens
QVTLKESGPVLVKPTETLTLTCTVSGFSLSNARMGVSWIRQPPGKALEWLAHIFSNDEKSYSTSLQSRLTISKDTSKSQVVLTLTNMDPVDTATYYCARIPGEDYYYYYGLDVWGQGTT

SEQ ID NO: 104 - protein, 103 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARER

SEQ ID NO: 105 - protein, 120 aa, Homo sapiens
QVQLQESGGGVVQPGRSLRLSCAASGFTFSNYFMHWVRQAPGKGLEWVAVIWHDGNNKNYADSVKGRFTISRDNSKNMLYLQMSSLRVEDTAVYYCARADWLGSNYFAYWGQGTLVTVSS

SEQ ID NO: 106 - protein, 126 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATALPFCSGGSCYHWYFDLWGRGTLVTVSS

SEQ ID NO: 107 - protein, 125 aa, Homo sapiens
QVQLQESGPGLVKPSQTLSLTCTVSGGSISSSYYWSWIRQPAGKGLEWIGRFIYSGSTSYKPSHQSRVTMAVGTSNNQFSLRLSSVTAADTAVYYCARRAAYAYDYIWGSTQYWGLAGTTVTVSS

SEQ ID NO: 108 - protein, 126 aa, Homo sapiens
QVQLQQSGPGLVKPSQTLSLTCAISGDSVSSNSAAWSWIRQSPSRGLEWLGRTYYRSKWYNDYAVSVKSRITINPDTSKNQFSLQLNSVTPEDTAVYYCARGSIVVVPAALSLVYYYYGMDVWGQG

SEQ ID NO: 109 - protein, 122 aa, Homo sapiens
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARALLRGPLVYYFDYWGQGTLVTVSS

SEQ ID NO: 110 - protein, 98 aa, Homo sapiens
EVQLVESGGGLVKPGGSLRLSCAASGFTFSDSYMNWVRQAPGKGLEWVSSISSSSTIYYADSVKGRFTISRDSAKNSLYLQMNSLRAEDTAVYYCARG

SEQ ID NO: 111 - protein, 123 aa, Homo sapiens
QVQLVQSGAEVKKPGSSVKVSCKASGGTFNRHAISWVRHSPGQGLEWMGGIIPIFGTANYAQKFQGRVTITADESTSTGYMELSSLRSEDTAVYYCAREGYPDTAMVNPFDYWGQGTLVTVSS

SEQ ID NO: 112 - protein, 125 aa, Homo sapiens
QVQLQESGPGLVKPSQTLSLTCTVSGGSINTGSYYWSWIRQPAGKGLEWSGGIHTSGSTNYPPSLKSRVTISVDTSKNQFSLKLSSVTGADTAVYYCARVPGYSWNNWTTKNYWGLAGTTVTVSS

SEQ ID NO: 113 - protein, 126 aa, Homo sapiens
EVQLVQSGAEVKKPGESLKISCKGSGYSFSTYWIAWLRQMPGKGLEWMGIIYAGDSETRYTPSFRGQVTISADKSTSTAYLQWSSLKASDTAMYYCARLEGRGYTGYALPYYFDYWGQGPLVTVSS

SEQ ID NO: 114 - protein, 124 aa, Homo sapiens
QVQLQESGPGLVKPSETLSLTCTISGGSISGDYWSWIRQPPGKGLEWIGHIYYSGNRYYTGTTNYNPSLKSRVTISVDTSRNQFSLRLTSVTAADTAVYYCARQRVSGRDLDYWGQGILVTVSS

SEQ ID NO: 115 - protein, 126 aa, Homo sapiens
QVHLVQSGAEVKKPGASLKVSCKVSGYTLTELSIHWVRQAPGKGLEWMGGFDREDGEAINAQKFQGRVTMTEDTSTDTFYMKLSSLRSEDTAVYFCAAGEALDCGGDCFFVAFNYWGQGTVVTISS

SEQ ID NO: 116 - protein, 124 aa, Homo sapiens
QVQLVQSGAEVKKPGASVKVSCKASGYTFTSYAMHWVRQAPGQRLECMGWINAGNGNTKYSQSFQGRVTITRDTSASTAYMELSSLRSEDTAVYYCARDPWGLTVTTWNWFDPWGQGTLVTVSS

SEQ ID NO: 117 - protein, 136 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGRTRVSVSTLYDSSGYYDFSGYYGMDVWGQGTTVTVSS

SEQ ID NO: 118 - protein, 129 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGADLGYCSGGSCYSFTMDVWGKGTTVTVSS

SEQ ID NO: 119 - protein, 125 aa, Homo sapiens
EMQLVESGGGLVQPGESLKLSCAASGFSFSGSTIHWVRLASGRGLEWVGRSRSKADNFMTSYAPSIKGKFIISRDDSSNMLYLQMNNLKTEDTAVYFCTRNFTSLDSTGNSFGPWGQGTLVTVSS

SEQ ID NO: 120 - protein, 121 aa, Homo sapiens
EEHLLESGGGLVQPGGSLRLSCAPSGFSFSSYAMSWVRQAPGKGLEWVANINHDDRRHYYLQSVKGRFTISRDNGRNLLYLEMNSLKVEDTAVYYCSRVIPGFPLDRFDIWGQGTMVTVSS

SEQ ID NO: 121 - protein, 123 aa, Homo sapiens
QVQLVESGGGLIQPGGSLRLSCAASGVTFSTYEMNWVRQAPGKGLEWVSYISNSGDTIYYSDSVKGRFSISRDNAKTSLYLQMNSLRVEDTAVYYCATGYYYDRGGNCNGDYWGQGTLVTVSS

SEQ ID NO: 122 - protein, 115 aa, Homo sapiens
QVQLVQSGGGLVQPGESLKLSCIASGFMFNIYNMTWVRQVPGKGLEWVSFISSRSDVIYYADSVKGRFTVSRHNANNSLYLQMNSLRVEDTAVYYCATNLTGDYWGQGTLVTVSS

SEQ ID NO: 123 - protein, 122 aa, Homo sapiens
EEHLVESGGDLVQPGGSLRLCCVASGFTFSNFAMNWVRQAPGQGLEWVSSISISGTIKYSDSVKGRFVVSRDNVKRSVFLQMNSLRGEDTAIYFCARNGSSADSPDYGFDVWGQGTTVTVTS

SEQ ID NO: 124 - protein, 119 aa, Homo sapiens
EVQLVQSRAAARKPRASVRVSCKASGYTFTNNALHWVRQAPGQSLEWMGWINTGNGITKYSQRFRDRVTITRDTSASTVYMEVHSLTPGDTAVYFCARWGDFYYYMDVWGQGTLVTVSS

SEQ ID NO: 125 - protein, 123 aa, Homo sapiens
QVQLVESGGGLIQPGGSLRLSCAAPGVTFSTYEMNWVRQAPGKGLEWVSYISNSGDTIYYSDSVKGRFSISRDNAKTSLYLQMNSLRVEDTAVYYCATGYYYDRGGNYNGDYWGQGTLVTVSS

SEQ ID NO: 126 - protein, 118 aa, Homo sapiens
QVQLQESGPGLVKPSETLSLICTVSGYSISSGYYWGWIRQPPGKGLEWIGNIYHNENTHYNPSLKSRLTMSVDTSKNQFSLNLSSVTAADTAIYYCARRMRAVAFDYWGQGTLVTVSS

SEQ ID NO: 127 - protein, 117 aa, Homo sapiens
QVQLQESGGGFVQPGGSLRLSCTGSGLSFSAYWIHWVRQAPGKGLVWVARLNSDGKKKNYAESVRGRFTASRDNAKNTVYLQMNSLRAEDTAVYYCVRENGNWFDSWGQGTLVTVSS

SEQ ID NO: 128 - protein, 119 aa, Homo sapiens
QVQLQESGPGLVKPSETLSLTCTVSGGSISSHGFFWVWIRRPPGKGLEWIGSVYYTGTTYYNSSLKSRVSISVDTSKNQFSLRLSSVTAADTAVYYCARRGDYRYFVPWGQGTLVTVSS

SEQ ID NO: 129 - protein, 119 aa, Homo sapiens
EEHLLESGGGLVQPGGSLRLSCAPSGFSFSSYAMSWVRQAPGKGLEWVSGISSSWGGTFYPDSVKGRFTISRNNSNNALYLQMDSLRADDTAVYYCARASYYYHSGYYYYMDVWGKGTT

SEQ ID NO: 130 - protein, 126 aa, Homo sapiens
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTRHVNDFWSGYPTLYYYGMDVWGQGPTV

SEQ ID NO: 131 - protein, 125 aa, Homo sapiens
QVHLVESGGGVVQPGRSLRLSCAASGFAFSTYGIHWVRQAPGKGLEWVAVISYDGVYKDYADSMKGRFTISRDNSKNTLYLQMNSLRPEDTAVYYCAKNLDNYGSGSYFYGMDVWGQGTTVSVSS

SEQ ID NO: 132 - protein, 99 aa, Homo sapiens
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARG

SEQ ID NO: 133 - protein, 125 aa, Homo sapiens
EVQLVQSGAEVKKPGASVKVSCKASGYSFISYGISWVRQAPGQGLEWMGWISAYNGNTNYAQKLQGRVTMTTDTSTSTAYMELRSLRSDDTAVYYCARSGRVGIFGVAGYYMDVWGKGTTVTVSS

SEQ ID NO: 134 - protein, 129 aa, Homo sapiens
QITLKESGPTLVKPTQTLTLTCTFSGFSLSTTAGGVGWIRQPPGKALEWLALIYWDDDKRYSPSLKSRLTITKDTSKNQVMLTMTNMDPVDTATYYCAHMPLFLGYYYGSGSYDWFDPWGRGTLVTVSS

SEQ ID NO: 135 - protein, 127 aa, Homo sapiens
EVQLVESGGGLVQPGGSLRLSCAASGFTFSSYAMSWVRQAPGKGLEWVSAISGSDGSTYYADSVKGRFTISRDNSKNTLYLQMNSLRAEDTAVYYCAKDRTPRNIVATKGMDAFDIWGQGTMVTVSS

SEQ ID NO: 136 - protein, 116 aa, Homo sapiens
QVTLKESGGELVQPGGSPRLSCSASGFTFSSLAMHWVRQAPGRGLEYVSISNGDGTSTTYADSVKGRFTTSRDNSKNTMYLQMNSLRPEDTAVYYCVRDVYGMDVWGRGTLVTVSS

SEQ ID NO: 137 - protein, 76 aa, Homo sapiens
EVQLVESGGGLVKPGGSLRLSRAASGFTFSDYYMNWVRQAPGKGLEWGSSITSSSTIYYADSVKGRFTISRDNAKN

SEQ ID NO: 138 - protein, 126 aa, Homo sapiens
QVQLQESGPGLVKPSQTLSLTCTVSGGSISSSYYWSWIRRPAGKGLEWIGRLFISGSTSYNPSLQSRVTMAVDASNNQFSLRLSSVTAADTAVYYCARGGAPYDYIAAWGSTYYWGLAGTTVTVSS

SEQ ID NO: 139 - protein, 124 aa, Homo sapiens
QVQLQESGPGLVKPSQTLSLTCTVSGGSISSRYYWSWIRQPAGKGLEWSGGIHTSGSTNYPPSLKSRVTISVDTSKNQFSLKLSSVTGADTAVYYCARVPGYSWNNWTTKNYWGLAGTTVTVSS

SEQ ID NO: 140 - protein, 124 aa, Homo sapiens
EVQLLESGGGLVQPGGSLRLSCAASGFTFGSYAFNWVRQAPGKGPEWISGISGSGESAHYADSVKGRFTISRDNSKKTLYLEMNSVTLEDTGVYYCATPILLPRSGRYSVRDYWGQGTLVTVSS

SEQ ID NO: 141 - protein, 98 aa, Homo sapiens
QVQLVQSGAEVKKPGESLKISYKGSGYSFTTYWIGWVRQMPGKGLEWMGIIFPGDSNTRYSPSFQGQVTISADKSINTAYLQWSSLKASDTAMYYCAV

SEQ ID NO: 142 - protein, 117 aa, Homo sapiens
QVQLQQSGPGLVKPSQTLSLTCAISGDSISSNSVAWNWIRQSPSRGLEWLGRTYCRSKWFNDYAVSVKSRITINPDTSKNQFSLHLNSVTPEDTGVYFCAREDRAIFHSGMDVWGQG

SEQ ID NO: 143 - protein, 126 aa, Homo sapiens
EVQLLESGGGLVQPGGSLRLSCAASGFTVNNYGMSWVRQAPGKGLEWVSTISASGVTTYYADSVKGRFTYSRDTSKNTLNLQMTSLRAEDTAVYYCAKDLRSYDPSGYYNDGFDIWGQGTMVTVSS

SEQ ID NO: 144 - protein, 124 aa, Homo sapiens
QVQLVQSGGGVVQPGRSLRLSCAASGFTFSGYAIHWVRQAPGKGLEWVAVISYDGSNKYYADSVKGRFTISRDNSKNTLYLQMNSLRAEDTAVYYCARSWDCSGDNCFQNIDIWGHGTMVTVSS

SEQ ID NO: 145 - protein, 128 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARARSVTVFGVVIWAVLNWFDPWGQGTLVTVSS

SEQ ID NO: 146 - protein, 120 aa, Homo sapiens
QVQLQESGGGLVKPGGSLRLSCAASGFTFSSYSMNWVRQAPGKGLEWVSSISSSSSYIYYADSVKGRFTISRDNAKNSLYLQMNSLRAEDTAVYYCARVHSGYDPYFDYWGQGTLVTVSS

SEQ ID NO: 147 - protein, 123 aa, Homo sapiens
QVQLVQSGGGLVQPGGSLRLSCAASGFTFSSYWMSWVRQAPGKGLEWVANIKQDGTDKYYVDSVKGRFTISRDNVKNSAYLEMNSLRVEDTAVYYCVRFPVKYSGHPDPLQYWGQGTLVTVSS

SEQ ID NO: 148 - protein, 51 aa, Homo sapiens
EVELVESGGGLVKPGESLTLSCEVSGFAFSKAWMTWVRQLQGRGWSGLAIL

SEQ ID NO: 149 - protein, 124 aa, Homo sapiens
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKDHGQWLVRGSQTGDYWGQGTLVTVSS

SEQ ID NO: 150 - protein, 121 aa, Homo sapiens
QVQLQESGPGLVKPSETLSLTCTVSGGYISSSSYYWGWTRQPPGKGLEWIGSIYYSGSTYYNPSLKSRVTLSVDTSKNQFSLKLSSVTAADTGVYYCSRLSGGYYSDFDYWSQGTLVTVSS

SEQ ID NO: 151 - protein, 121 aa, Homo sapiens
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARDPMGIYSGYEDVWGKGTTVTVSS

SEQ ID NO: 152 - protein, 110 aa, Homo sapiens
GPGLVKPSETLSLTCTVSGGSISSSSYYWGWIRQPPGKGLEWIGSIYYSGSTYYNPSLKSRVTISVDTSRNQFSLKLSSVTAADTVVYYCARHPSMVVVADFDYWGQGTL

SEQ ID NO: 153 - protein, 116 aa, Homo sapiens
QVQLQESGPGLVKPSETLSLTCTVSGDSISSSYWSWIRQAPGRGLEWIGYISSNGAPSTTPPSKSRVTMSLDTSRNQLSLKLTSVIAADTALYYCAGFLGAAFDYWGQGTLVTVSS

SEQ ID NO: 154 - protein, 125 aa, Homo sapiens
EMQLVESGGGLVQPGESLKLSCAASGFSFSGSTIHWVRQASGRGLEWVGRSRSKADNFMTSYAPSIKGKFIISRDDSSNMLYLQMNNLKTEDTAVYFCTRNFTSLDSTGNSFGPWGQGTLVTVSS

SEQ ID NO: 155 - protein, 102 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARV

SEQ ID NO: 156 - protein, 99 aa, Homo sapiens
EVQLVESGGGLVKPGGSLRLSCAASGFTFSDYYMNWVRQAPGKGLEWVSSISSSSTIYYADSVKGRFTISRDNAKNSLYLQMNSLRAEDTAVYYCARDP

SEQ ID NO: 157 - protein, 121 aa, Homo sapiens
QVQLVQSGAEVKKPGASVKVSCKASGYTFTSYGISWVRRAPGQGLEWMGWISAYNGNRNYAQKVQGRVTMTTDTSTSTAYMELRSLRSDDTAVYYCARDSGFGERGWIDYWGQGTLVTVSS

SEQ ID NO: 158 - protein, 124 aa, Homo sapiens
EEHLLESGGGLVQPGGSLRLSCAPSGFSFSSYAMSWVRQAPGKGLEWVSGISSSGGGTFYADSVKGRFTISRDNSNNALYLQMDSLRADDTAVYYCARASYYYHSGYYYYMDVWGKGTTVTVSS

SEQ ID NO: 159 - protein, 125 aa, Homo sapiens
QVQLQESGGGLVQPGGSLRLSCAASGFTFSSYAMSWVRQAPGKKLEWVSTFSGGRDTTYYADSVRGRFTISRDNSRNTLYLQMNSLRADDTAVYFCSKGSRGTCYGAYCYYFDHWGQGALVTVSS

SEQ ID NO: 160 - protein, 122 aa, Homo sapiens
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGERKRGEFDYWGQGTLVTVSS

SEQ ID NO: 161 - protein, 123 aa, Homo sapiens
EVQLVESGGGLVQPGGSLRLSCAASGFTFTDYWMHWVRQLPGKGLVWVARINGDGSSATYTDSVQGRFTVSRDNAQNTLYLQMNSLRAEDTAVYYCARDHRHCSGGSCLVDYWGQGSLVTVSS

SEQ ID NO: 162 - protein, 117 aa, Homo sapiens
EMQLVESGGAFVQPGGSLRLSCAASGFNFSDSTIHWVRQASGKSLEWVGHIEEKSKKYATIFRASVKGRFIISRDDSKNTAFLQMDSLRPDDTALYYCTPPPEVESLRSWGRGTLVT

SEQ ID NO: 163 - protein, 126 aa, Homo sapiens
QITLKESGPTLVKPTQTLTLTCTVSGSSLSTSGVGVGWLRQPPGKALEWLALIYWDDDSRYSPSLEGRLTITKDTSKNQVVLTMTDMDPVDTGTYYCAHRHYYDFWSGYYDWFDPWGQGIPVTVSS

SEQ ID NO: 164 - protein, 121 aa, Homo sapiens
QVQLVESGGGVVQPGRSLRLSCAASGFTFSSFGMHWVRQAPGKGLEWVATIAFHGDIKFYADSVRGRFTISRDNSNNTLYLQMNSLRVDDTAAYYCAKDCRDWGGPAQFGHWGKGTLVTVS

SEQ ID NO: 165 - protein, 118 aa, Homo sapiens
QVQLQESGPGLVKPSETLSLICTVSGYSISSGYYWGWIRQPPGKGLEWIGNIYHNENTHYNPSLKSRLTMSVDTSKNQFSLNLSSVTAADTAVYYCARRMPAVAFDYWGQGTLVTVSS

SEQ ID NO: 166 - protein, 125 aa, Homo sapiens
QVQLQESGPGLVKPSQTLSLTCTVSGGSINSGSYSWSWIRQPAGKGLEWIGRIYTSGSTNYNPSLKSRVTMAVATSKSQFSLRLSAVTAADTAVYYCARLAAPIAYNWGSTQYWGLAGTTVTVSS

SEQ ID NO: 167 - protein, 134 aa, Homo sapiens
QVQLQESGPGLVKPSQTLSLTCTVSGASIIRGDYYWSWIRQSPGKGLEWIGYIYSSGTTFYNPSLRSRPAISIDTSKNQFSLKLSSVTAADTAVYYCARANIEVVPAIAINEKHSFYFYSMDVWGQGTTVTVSS

SEQ ID NO: 168 - protein, 127 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGKGITMTSYCYYYGMDVWGQGTTVTVSS

SEQ ID NO: 169 - protein, 123 aa, Homo sapiens
QMQLVQSGVEVKKPGESLRISCKGSGYSFTSYWIGWVRQMPGKGLEWMGIIYPGDSETRYSPSFQGQVTISADKSISTAYLRWSSLKASDTAIYYCASMGYSIVVAGTYFDYWGQGTLVTVSS

SEQ ID NO: 170 - protein, 114 aa, Homo sapiens
QVQLQQSGPGLVEPSQTLSLTCAISGDSVSSNGAAWNWLRQSPSRGLEWPGRTYSRSKWYKEYAVSVQSRITINSDTSKNQFFLQLNSVTPEDTAVYFCTRGFLRSGMDVWGQG

SEQ ID NO: 171 - protein, 118 aa, Homo sapiens
QVQLQQSGPGLVKPSQTLSLTCAISGDSVSSNSAAWNWIRQSPSRGLEWLGRTYYRSKWYNDYAVSVESRITINPDTSKNQFSLQLNSVTPEDTAVYYCARDRGSGWYRGAFDIWGQG

SEQ ID NO: 172 - protein, 118 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREPSGEQLVQNFDYWGQG

SEQ ID NO: 173 - protein, 125 aa, Homo sapiens; identical to SEQ ID NO: 154

SEQ ID NO: 174 - protein, 123 aa, Homo sapiens; identical to SEQ ID NO: 9

SEQ ID NO: 175 - protein, 121 aa, Homo sapiens
LVQLQESGPGLVKPSETLSLTCIVSGASIRSYYWSWIRQPPGKGLEWIAYIYNSGSTNYNPSLRSRVTISLDTSKNQFSLKLSSVTAADTAVYYCASSDFGYTAYDEIWAYHFDHWGQGTL

SEQ ID NO: 176 - protein, 121 aa, Homo sapiens
QVQLQESGGGLVQPGGSLRLSCAASGFTFSSYAMSWVRQAPGKGLEWVSAISGGGGSTYSADSVKGRFTISRDNSKNTLYLQMNSLRAEDTAVYYCARPSTSGTTLGWFDPWGQGTLVTVS

SEQ ID NO: 177 - protein, 123 aa, Homo sapiens
QVQLVESGGGLIQPGGSLRLSCAASGVTFSTYEMNWVRQAPGKGLEWVSYISKGGDTIYYSDSVKGRSIISRDNAKTSLYLQMNSLRVEDTAVYYCATGYYYDSGGNYNGDYWGQGTLVTVSS

SEQ ID NO: 178 - protein, 117 aa, Homo sapiens
EMQLVESGGAFVQPGGSLKLSCAASGFNFSDSTIHWVRQASGKSLEWVGHIENKTNNYATVYRASVKGRFNISRDDSKNMTFLQMDSLRPDDTALYYCTPPPEVESLRSWGRGTLVT

SEQ ID NO: 179 - protein, 120 aa, Homo sapiens
QVQLQESGPGLVKPSQTLSLTCTVSGGSISSSYYWSWIRQPAGKGLEWMGSSYYNPSLKSRVTISVDTSKNQFSLKLSSVTAADTAVYYCARRAAPIAYDYIWGSTQYWGLAGTTVTVSS

SEQ ID NO: 180 - protein, 101 aa, Homo sapiens
EVQLLESGGGLVQPGGSLRLSCAASGFTFSSYAMSWVRQAPGKGLEWVSAIDGGSFNTYYADSVKGRFTISRDNSKNTLYLQMNSLRAEDTARYYCGGGTN

SEQ ID NO: 181 - protein, 126 aa, Homo sapiens
QITLKESGPTLVKPSQTLTLTCSVSGSSLSTSGVGVGWLCQPPGKALEWLALIYWDDDARYNPSLEGTLTITKDTSKHQVILTMTDMDPVDTATYYCAHRHYYDFWSGYYDWFDPWGQGTLVTVSS

SEQ ID NO: 182 - protein, 123 aa, Homo sapiens
QVRLVESGGGLIQTGGSLRLSCAASGVTFSTYEMNWVRQAPGKGLEWVSYISKGGDTIYYSDSVKGRFIISRDNAKTSLYLQMNSLRVEDTAVYYCATGYYYDSGGNYNGDYWGQGTLVTVSS

SEQ ID NO: 183 - protein, 123 aa, Homo sapiens
QVQLQESGAEVRKPGASVKVSCKASGYTLTTYYMHWVRQAPGQGLEWMGLINPSGGSGGNIHKFQGRLTMTRDTSTSTVYMEMSSLRSEDTAVYFCARELVHGSTWLKAFDIWGQGTMVTVSS

SEQ ID NO: 184 - protein, 118 aa, Homo sapiens
QVQLQESGGGLVQPGGSLRLSCAASGFTFSDHYMDWVRQAPGKGLEWVGQTRNEANSYAIKYAASVKGRFTISRDDSKNSVYLQMNSLQSEDTAVYYCLVWFSGRSYWGQGTLVTVSS

SEQ ID NO: 185 - protein, 121 aa, Homo sapiens
QVQLVESGGGLVKPGGSLRLSCAASGFIFSDYYMNWIRQAPGKGLEWISYISGSSTFTNYPDSVKGRFTISRDNDRNSLYLQMSSLRSEDTAVYYCARGSAPGIGAAGDFWGQGILVTVSS

SEQ ID NO: 186 - protein, 126 aa, Homo sapiens
QITLKESGPTLVNPTQTLTLTCIVSGSSLSARGVGVGWLRQPPGKALEWLALIYWDGDAGYSPSLEGRLTITKDTSKNQVVLTMTNMEPVDTATYYCAHRHYHDFWFGYYDWFDPWGPGTLVTVSS

SEQ ID NO: 187 - protein, 103 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREQ

SEQ ID NO: 188 - protein, 124 aa, Homo sapiens
QVQLQQSGPGLVKPSQTLSLTCAISGDSVSSNSVSWNWIRQSPSRGLEWLGRTYYRSKWYNDSAVSVKSRITINPDTSKNQFSLQLNSVTPEDTAVYYCVRARPGTTGGGMDVWGQGTTVTVSS

SEQ ID NO: 189 - protein, 125 aa, Homo sapiens
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDRPYDFWSGYKDAFDIWGQGTMVTVSS

SEQ ID NO: 190 - protein, 113 aa, Homo sapiens
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVPWTFGGGTKLEIKR

SEQ ID NO: 191 - protein, 108 aa, Homo sapiens
DIQMTQSPSSLSASVGDRVTITCQASQDISNYLIWYQQKPGKAPKLLIYDASNLETGVPSRFSGSGSGTDFTFTISSLQPEDIATYYCQQYHNLPPYTFGPGTKLEIK

SEQ ID NO: 192 - protein, 113 aa, Homo sapiens
DILVTQSPGSLAVSLGERATINCKSSQSILYSSNNKNYLAWYQQKAGQPPKLLISWASTRESGVPDRFSVSGSGADFTLTISSLQAEDVAVYYCQQYYNTPRTFGQGTKLEIK

SEQ ID NO: 193 - protein, 112 aa, Homo sapiens
DVVMTQSPLSLPVTLGQPASISCRSSQSLVYSDGNTHLNWFQQRPGQSPRRLIYKVSNRDSGVPDRFSGSGSGTDFTLKISRVEAEDVGVYYCMQGTHWPYTFGQGTKLEIK

SEQ ID NO: 194 - protein, 106 aa, Homo sapiens
DIQMTQSPSSLSASVGDRVTITCQTSQDISKYLAWYQQKPGKVPKLLIYAASTLQGVPSRFSGGGSGTDFTFTISSLQPEDIATYYCQQYDTLPLTFGGGTKVEIK

SEQ ID NO: 195 - protein, 107 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 196 - protein, 113 aa, Homo sapiens
DIVMTQSPDSLAVSLGERATINCKSSQSVLYSFNNNDCLAWYQQKPGQPPKLLIHWAFSREAGVPDRFSGSGSGTDFTLTISSLQAEDVAVYYCHQYCTAPLAFGGGTKVEIK

SEQ ID NO: 197 - protein, 108 aa, Homo sapiens
EIVLTQSPATLSLSPGERGTLSCRASQSVSSYLAWYQQKPGQAPRLLIYDASIRATDIPARFSGSGSGTDFTLTISSLEPDDFAVYYCQQSSTWPPYTFGQGTKLEIK

SEQ ID NO: 198 - protein, 101 aa, Homo sapiens
EIVMTQSPVNLSMSAGERVTLSCRASQSVGSNIAWYQQKPGQAPRLFIYDASKTATGISARFSGSGSGTDFTLTITSLQSEDFAVYYCQQYHNPNTLLARG

SEQ ID NO: 199 - protein, 108 aa, Homo sapiens
DIQMTQSPGTLSLSPGERATLSCRASQSVSSSYLAWYQQKPGQAPRLIIYGASSRATGIPDRFSGSGSGTDFTLTISRLEPEDFAVYYCQQYGSSPRTFGQGTKVEIK

SEQ ID NO: 200 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 201 - protein, 108 aa, Homo sapiens
DIVMTQSPGTLSLSPGERATLSCRASQSVSNNYLAWYQQKPGQAPRLLIYAASSRATGIPDRFSGSGSGTDFTLTISRLEPEDFAVYYCQQYGSSLRTFGQGTKVDIK

SEQ ID NO: 202 - protein, 112 aa, Homo sapiens
ETQMTQSPDSLAVSLGERATINCKSSQSVLYSPNSKNYLAWYQQKPGQPPKLLIYWASTRESGVPDRFSGSGSGTDFTLTISSLQAEDVAVYYCQQYLSSRTFGPGTKVEIK

SEQ ID NO: 203 - protein, 106 aa, Homo sapiens
EIVLTQSPATLSLSPGERATLSCRASQGVSSYLAWYQQKPGQAPRLLIYDASNRATGIPARFSGSGSGTDFTLTISSLEPEDFAAYYCQQRSNTPTFGGGTKVEIK

SEQ ID NO: 204 - protein, 107 aa, Homo sapiens
DIQMTQSPSSLSASVGDRVTITCRASQDITNCLTWYQQKPGKAPKLLIYDASNLETGVPSRFSGSGSGTDFTFTISSLQPEDIATYYCQQYDSLPITFGQGTKVEVK

SEQ ID NO: 205 - protein, 110 aa, Homo sapiens
DIVMTQSPDSLAVSLGERATINCNASQSVLYSSNNKNYLAWYQQKPGQPPKLLIYWASTRESGVPDRFSGSGSGTDFTLTISSLQAEDVAVYYCQQYYSTLRGRSAKGPR

SEQ ID NO: 206 - protein, 107 aa, Homo sapiens
DIEMTQSPSSLSASVGDRVTITCRASQSISSYLNWYQRKPGKAPKLLIYAASSLQSGVPSRFSAGVSGTDFTLTISSLQSEDFATYYCQQSYSPPYTFGQGTTLEIK

SEQ ID NO: 207 - protein, 107 aa, Homo sapiens
DIEMTQSPSTLSASVGDRVTITCRASQSISSWLAWYQQKPGKAPKLLIYKASRLESGVPSRFSGSGSGTDFTLTISSLQPDDFATYYCQQYNSYSRTFGQGTKVEIK

SEQ ID NO: 208 - protein, 106 aa, Homo sapiens
EIVLTQSPLSLPVTPGQPATFSCKSSQSEGKTYLAWLQQKPGQSPQLLIYDVSNLQCGVPDRFSGSGRDFTLKISRVEAEDFGVYYCMQGTHWSPTFGQGTKVEIK

SEQ ID NO: 209 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPQTFGQGTKVEIK

SEQ ID NO: 210 - protein, 107 aa, Homo sapiens
ELVLTQSPATLSLSPGERATLSCRASQSVSSNLAWYQQKPGQAPRLLIYGASTRATGIPARFSGSGSGTEFTLTISSLQSEDFAVYYCQQGLRAPFTFGQGTKVEIK

SEQ ID NO: 211 - protein, 108 aa, Homo sapiens
EIVMTQSPATLSVSPGERATLSCRASQSVNSNLAWYQQKPGQAPRLLIYGASTRATGIPVRISGSGSGTEFTLTISSLQSEDFAVYYCQEYNNWPLWTFGQGTKVEIK

SEQ ID NO: 212 - protein, 107 aa, Homo sapiens
DIVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 213 - protein, 113 aa, Homo sapiens
ESLVTQSPDSLPVSLGERATINCKSSQNVLFSFNNKEALAWYQQKPGQPPKLLIYWASTRESGVPDRFSGSGSGTDFTLTISSLQPEDVATYYCQKYNSAPFTFGGGTKVEIK

SEQ ID NO: 214 - protein, 107 aa, Homo sapiens
DIQMTQSPSSLSASVGDRVTITCQASQDIAKFLNWYQQRPGKVPKLLIYDASNLAIGVPSRFSGSGSGTDFTFTISSLQPEDIATYYCQHYDDFPISFGPGTRLETK

SEQ ID NO: 215 - protein, 112 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYDTIPTFGGGTKVEIK

SEQ ID NO: 216 - protein, 113 aa, Homo sapiens
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 217 - protein, 108 aa, Homo sapiens; identical to SEQ ID NO: 191

SEQ ID NO: 218 - protein, 112 aa, Homo sapiens
DIVMTQTPLSSPVTLGQPASISCRSSQSLVHSDGNTYLSWLQQRPGQPPRLLLYKISNRFSGVPDRFSGSGAGTDFTLEINRVEAEDVGVYYCMQAKEFPRTFGQGTKLEIK

SEQ ID NO: 219 - protein, 107 aa, Homo sapiens
DIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIK

SEQ ID NO: 220 - protein, 107 aa, Homo sapiens
DIQMTQSPSSLSASVGDRVTITCQASQDISNYLNWYQQKPGKAPKLLIYDASNLETGVPSRISGSGSGTDFTFTISSLQPEDIATYYCQQYDNLPLTFGGGTKVEIK

SEQ ID NO: 221 - protein, 108 aa, Homo sapiens; identical to SEQ ID NO: 191

SEQ ID NO: 222 - protein, 106 aa, Homo sapiens
GIQMTQSPSTLSASIGDRVSITCRANQSVNIWLAWYQQRPGRAPKLLIYGASSLESGVSPRFSGRGFGTEFTLTISSLQPGDFATYFCQQYDSDSSFGQGTKLEMR

SEQ ID NO: 223 - protein, 107 aa, Homo sapiens
DIQMTQSPSSLSASVRDRVRITCRASQGIRNDLGWYQQKPGKAPKRLIYAASTLQSGVPSRFSGSGSGTEFTLTISSLQPEDSATYYCLQHNSYPRTFGQGTKVEIK

SEQ ID NO: 224 - protein, 107 aa, Homo sapiens
DIEMTQSPPSLSASVGDRVTITCRASQSINSYLNWYQQKPGKAPKLLIYAASSLESGVPSRFSGSESGTDFTLTISSLQPEDFATYYCQQSYSSPSAFGQGTKVEIK

SEQ ID NO: 225 - protein, 108 aa, Homo sapiens
EIVMTQSPATLSVSPGERATLSCRASQSVSNNLAWYQQKPGQPPRLLIYGASTRATGIPARFSGSGSGTEFTLTISRLQSEDFAVYYCQQYNNWPPWTFGQGTRVEIK

SEQ ID NO: 226 - protein, 107 aa, Homo sapiens
ELVLTQSPGTLSVSPGERATLSCRASQSVSSNLAWYQQKPGQAPRLLIYGASTRPTGIPARFSGSGSGTEFTLTISSLQSEDFATYYCQHPRRLPLTFGQGTKVEIK

SEQ ID NO: 227 - protein, 108 aa, Homo sapiens; identical to SEQ ID NO: 191

SEQ ID NO: 228 - protein, 112 aa, Homo sapiens
DVVMTQSPLSLPVTLGQPASISCRSSQSLVYSDRNTYLNWFQQRPGQSPRRLIYKVSNRDSGVPDRFSGSGSGTDFTLKISRVEAEDVGVYYCMQGTHWSWTFGQGTKVEIK

SEQ ID NO: 229 - protein, 107 aa, Homo sapiens
DIQITHSPSSLSASVGDRVTITCRASQGISNYLAWFQQKPGKAPKSLLYNPSSLQSGVPSRFSGSGSGTDFTLTINNLQPEDFATYYCQQLLSYPRTFGQGTKLEIK

SEQ ID NO: 230 - protein, 108 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPPSTFGQGTKVEIK

SEQ ID NO: 231 - protein, 106 aa, Homo sapiens
  misc_feature (63)..(63): Xaa can be any naturally occurring amino acid
DIQMTQSPSTLSASVGDTVTITCRASQNINYWLAWYQQKPGKAPTLLIFKASIVQTGVPSRFXGXGSGTKFTLTISGLQPEDXATYHCQEYXSSSWTFGRGTTVGR

SEQ ID NO: 232 - protein, 107 aa, Homo sapiens
ELQMTQSPSSLSASVGDRATVSCQASQSIYNYLNWYQQKPGKAPKFLTYRASSLQRGMPSQFSGSGYGRDFTLTVSSLQPDDFATYYCQQGVRLPYTFGQGTKVEIK

SEQ ID NO: 233 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRWTFGQGTKVEIK

SEQ ID NO: 234 - protein, 108 aa, Homo sapiens
ELVLTQSPGTLSLSPGERATLSCGASQSVSSSYLAWYQQKPGLAPRLLIYDASSRATGIPDRFSGSGSGTDFTLTISRLEPEDFAAYYCQHRNQVPFTFGQGTKVEIK

SEQ ID NO: 235 - protein, 108 aa, Homo sapiens; identical to SEQ ID NO: 191

SEQ ID NO: 236 - protein, 108 aa, Homo sapiens; identical to SEQ ID NO: 191

SEQ ID NO: 237 - protein, 108 aa, Homo sapiens
EIVLTQSPGTLSLSPGERATLSCRASQSVSRSQLAWYQQSPGQAPRLLIYDASSRATGIPDRFSGSGSGTDFTLTISRLEPEDFAVYYCQQCSSSPWTFGQGTKVEIK

SEQ ID NO: 238 - protein, 107 aa, Homo sapiens
DIQLTQSPSFLSASVGDRVTITCRASQDIRNYLTWYQQKPGKAPKVLIYPASNLETGVPSRFSGSGSGTEFTLTIISLQPEDFATYYCQQLISYPSTFGGGTKVEIK

SEQ ID NO: 239 - protein, 112 aa, Homo sapiens
DIVMTQTPLSLPVTLGQPASISCRSSRGLVHSDGNTYLNWFQQRPGQSPRRLIYKVSNRDSGVPDRFSGSGSGADFTLKISRVEAEDVGVYYCMQSIHWPWTFGQGTKVEIK

SEQ ID NO: 240 - protein, 108 aa, Homo sapiens
EIVMTQSPGTLSLSPGERATLSCRASQSVSSSYLAWYQQKPGQAPRLLIYGASSRATGVPDRFSGSGSGTDFTLTISRLEPEDFAVYYCQQYRSSPYTFGQGTKLEIK

SEQ ID NO: 241 - protein, 107 aa, Homo sapiens
  misc_feature (63)..(63): Xaa can be any naturally occurring amino acid
DIQMTQSPSTLSASVGDTVTITCRASQNINYWLAWYQQKPGKAPTLLIFKASIVQTGVPSRFXGSGSGTKFTLTISGLQPEDFATYHCQEYHSSSWTFGQGTTVERK

SEQ ID NO: 242 - protein, 107 aa, Homo sapiens
DIQMTQSPSSLSASVGDRVTITCQASQDIAKFLNWYQQRPGKVPKLLIYDASNLAIGVPSRFSGSGSGTDFTFTISSLQPEDIATYYCRHYDDFPISFGPGTRLETK

SEQ ID NO: 243 - protein, 108 aa, Homo sapiens
DILMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 244 - protein, 112 aa, Homo sapiens
DIVMTQSPLSSPVTLGQPASISCRSSQSLVHSDGSTYLSWLQQRPGQPPRLLLHKISNRFSGVPDRFSGSGAGTDFTLKISRVEAEDVGVYYCMQATQLPLTFGGGTKVEIK

SEQ ID NO: 245 - protein, 106 aa, Homo sapiens
DIVMTQSPSTLSASVGDRVTITCRASQSISNWLAWYQQKPGKAPKLLIYYASTLETGVPPRFSGSGSGTEFALTISSLQPDDFATYYCQQYDSYSTFGQGTKVEVK

SEQ ID NO: 246 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 247 - protein, 107 aa, Homo sapiens
DIVMTQSLSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIK

SEQ ID NO: 248 - protein, 107 aa, Homo sapiens
DIQMTQSPSTLSASVGDTVTITCRASQNINYWLAWYQQKPGKAPTLLIFKASIVQTGVPSRFSGSGSGTKFTLTISGLQPEDFATYHCQEYHSSSWTFGQGTTVERK

SEQ ID NO: 249 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGQGTKVEIK

SEQ ID NO: 250 - protein, 108 aa, Homo sapiens; identical to SEQ ID NO: 191

SEQ ID NO: 251 - protein, 108 aa, Homo sapiens; identical to SEQ ID NO: 237

SEQ ID NO: 252 - protein, 109 aa, Homo sapiens
EIVLTQSPGTLSLSPGERATLSCRAGQSVSSSYLASYQQKPGQAPRLLIYGASSRATGIPDRFSGSGSGTDFTLTISRLEPEDFAVYYCQQYGSSSSATFGQGTKVEIK

SEQ ID NO: 253 - protein, 113 aa, Homo sapiens
DIVMSQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELK

SEQ ID NO: 254 - protein, 109 aa, Homo sapiens
DIQMTQSPSSLSASVGDRVTSTCQASQDISNYLNWYQQKPGKAPKLLIYDASNLETGVPSRFSGSGSGTDFTFTISSLQPEDIATYYCQQYDNLPPGVTFGPGTKVDIK

SEQ ID NO: 255 - protein, 106 aa, Homo sapiens
DIQMTQSPSSLSASAGDRVSFTCRTSQNIGNYLSWYQQKPGRAPKLLIYDASTLQDGVPARFSGSGSGTDFTLTISSLQPEDFATYYCQQTSSVPITFGQGTRLEM

SEQ ID NO: 256 - protein, 107 aa, Homo sapiens
DIQMTQSPSSLSASVGDRVTITCRASQSISNYLNWYQQKPGKAPKLLIYAASSLQSGVTSRFSGSGSGTDFTLTISSLQPEDSATYYCQQSYSTLITFGQGTRLEIK

SEQ ID NO: 257 - protein, 112 aa, Homo sapiens; identical to SEQ ID NO: 209

SEQ ID NO: 258 - protein, 108 aa, Homo sapiens
EIVLTQSPGTLSLSPGERATLSCRASQSVSRSQLAWYQQSPGQAPRLLIYDASSRATGIPDRFSGSGSGTDFTLTISRLEPEDFAVYYCQQCSSSPWMFGQGTKVEIK

SEQ ID NO: 259 - protein, 108 aa, Homo sapiens
ELVLTQSPGTLSLSPGERATLSCRASQSVSSSYLAWYQQKPGQAPRLLIYGASSRATGIPDRFSGSGSGTDFTLTISRLEPEDFAVYYCQQYNHVPGTFGQGTKVEIK

SEQ ID NO: 260 - protein, 113 aa, Homo sapiens; identical to SEQ ID NO: 253

SEQ ID NO: 261 - protein, 108 aa, Homo sapiens
EIVLTQSPGTLSLSQGERVTLSCRASQTINSHYLAWYQQKPGQAPRLLIYDASSRATGVPDRFSGSGSGTDFILTISGLEPEDFAVYYCQQYGTSPLTFGGGTKVEIK

SEQ ID NO: 262 - protein, 113 aa, Homo sapiens; identical to SEQ ID NO: 249

SEQ ID NO: 263 - protein, 107 aa, Homo sapiens
EIVLTQSPATLSLSPGQRATLSCRASQSVSSYLAWYQQKPGQAPRLLIYDASNRATGIPARFSGSGSGTDFTLTISSLEPEDFAVYYCQQRSNWPYTFGQGTKLEIK

SEQ ID NO: 264 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTWTFGQGTKVEIK

SEQ ID NO: 265 - protein, 106 aa, Homo sapiens
DIQMTQSPSSLSASVGDRVTITCRASQSISTWLAWYQQKPGKAPKLLIQTASRLESGVPSRFSGSGSETDFTLTISSLQPEDFATYYCQQYNSLWTFGQGTKVEIK

SEQ ID NO: 266 - protein, 107 aa, Homo sapiens
DIVMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 267 - protein, 113 aa, Homo sapiens
DIVMTQSPDSLVVSLGERATINCKSSQSLLYSSNNKTYLAWYQQKPGQPPKLLIKWASTRESGVPDRFSGSGSGTDFTLTISSLQAEDVAVFYCQQYYTSPLTFGGGTKVEIK

SEQ ID NO: 268 - protein, 97 aa, Homo sapiens
EIVLTQSPATLSLSPGERATLSCGASQSVSSSYLAWYQQKPRLAPRLLIYDASSRATGIPDRFSGSGSGTDFTLTISRLEPEDFAVYYCQQYGSSRP

SEQ ID NO: 269 - protein, 107 aa, Homo sapiens; identical to SEQ ID NO: 248

SEQ ID NO: 270 - protein, 106 aa, Homo sapiens
EIVMTQSPATLSVSPGERVTLSCRASQTIDKNLDWIQKKPGQAPRLLIYRASTRATGVPARFSGSGSGAEFTLTISSLQSEDSAVYYCLQHSFWRTFGQGTNLEIK

SEQ ID NO: 271 - protein, 106 aa, Homo sapiens
AIRMTQSPFSLSAAAGDRVTITCWASQGISGYLASYQQPAKAPKFFIYDAPILQSGVPSRFSGSGSGTECTLTISSLQPEDFATYHCQQYFSIQFTFGGGTKVEIK

SEQ ID NO: 272 - protein, 113 aa, Homo sapiens
DIVMTQTPLSLSVTPGQPASISCKSSQSLLHSDGKTYLYWYLQKPGQSPQLLIYEVFNRFSGVPDRFSGSGSGTDFTLKISRVEAEDVGVYYCMQSIQLPRWTFGQGTKVEIK

SEQ ID NO: 273 - protein, 107 aa, Homo sapiens
  misc_feature (42)..(42): Xaa can be any naturally occurring amino acid
DIQMTQSPSTLSASVGDTVTITCRASHNINYWLAWYQQKPGXAPTLLIFKASILQTGVPSRFXGSGSGTKFTLTISGLQPEDFATYHCQXYHSSSWTFGQGTTVERK

SEQ ID NO: 274 - protein, 108 aa, Homo sapiens
EVVLTQLPGTLSLSPGERATLSCRASQSVSSTYLAWYHQKPGQAPRLLIYGASSRATGIPDRFSGSGSGTDFTLTISRLEPEDFAVYYCQQYGSSPVTFGGGTKLEIK

SEQ ID NO: 275 - protein, 107 aa, Homo sapiens
IVLTQSPGTLSLSPGERATLSCRASQSVSSNELAWYQQKPGQAPRLLISGASRRATGIPDRFSGSGSGTDFTLTISRLEPEDFAMYYCQQYGSTPRTFGQGTKVEIK

SEQ ID NO: 276 - protein, 107 aa, Homo sapiens
DIQMTQSPSSLSASVGDRVTITCRASQSIGSFLHWYQQKPGKGPKLLISAASSLQSGVPSRFSGSGSGTDFTLTISSLQPEDFATYYCQQSFSTLYTFGQGTKLEIK

SEQ ID NO: 277 - protein, 106 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPAHFRRR

SEQ ID NO: 278 - protein, 109 aa, Homo sapiens
KIVLTQSPGTLSLSPGERATLSCRASQSVSGDYLAWYQQKPGQAPRLLVYGASSRATGIPDRISGSGSGTDFILTISRLEPEDFAVYYCQQYGGSLPITFGGGTKVEIK

SEQ ID NO: 279 - protein, 108 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 280 - protein, 112 aa, Homo sapiens
DIVMTQSPLSLPVTLGQPASISCRSSQGLVYSDGDTYLNWFQQRPGQSPRRLIYKVSNRDSGVPDRFSGGGSGTDFTLKISRVEAEDVGVYYCMQGTHWPYTFGQGTKLEIK

SEQ ID NO: 281 - protein, 107 aa, Homo sapiens; identical to SEQ ID NO: 214

SEQ ID NO: 282 - protein, 107 aa, Homo sapiens
ELQMTQSPSSLSASVGGRVTITCRASQGISNNLNWYQQKPGKTPKLLIYAASSFQSGVPSRFSGSGSGTDFTLTISSLQPEDFATYYCQQPLSSPHTFGQGTKVEIK

SEQ ID NO: 283 - protein, 107 aa, Homo sapiens; identical to SEQ ID NO: 214

SEQ ID NO: 284 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPLTFGGGTKVEIK

SEQ ID NO: 285 - protein, 107 aa, Homo sapiens
  misc_feature (90)..(90): Xaa can be any naturally occurring amino acid
DIQMTQSPSTLSASVGDTVTITCRASHNINYWLAWYQQKPGKAPTLLIFKASIVQTGVPSRFSGSGSGTKFTLTIIGLQPEDFATYHCQXYHSSSWTFGQGTTVERK

SEQ ID NO: 286 - protein, 107 aa, Homo sapiens
DIQMTQSPSSLSASVGDRVTITCRASQSISNYLSWYQQKPGKAPKLLIYAASSLQSGVTSRFSGSGSGTDFTLTISSLQPEDSATYYCQQSYSTLITFGQGTRLEIK

SEQ ID NO: 287 - protein, 107 aa, Homo sapiens
DIQMTQSPSSLSASVGDRVTITCRASQSISSYLNWYQQKLGKAPKLLIYSASSLQSGVPSTFSGSGSGTDFTLTISSLQPEDFAIYYCQQSYSTPWTFGQGTKVEIK

SEQ ID NO: 288 - protein, 107 aa, Homo sapiens
DIQMTQSPSSLSASVGDRVTITCQASQDITNYLTWYQQKPGKAPKLLIFDASNLETGVPSRFSGSGSGTDFTFTISSLQPEDIATYYCQQYDSLPYTFGQGTKVEVK

SEQ ID NO: 289 - protein, 107 aa, Homo sapiens
DIQLTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIK

SEQ ID NO: 290 - protein, 111 aa, Homo sapiens
DIVMTQSPLSLPVTLGQPASISCRSSQGLVYSDGDTYLNWFQQRPGQSPRRLIYKVSNRDSGVPDRFSGGGSGTDFTLKISRVEAEDVGVYYCCRSHWPYTFGQGTKLEIK

SEQ ID NO: 291 - protein, 107 aa, Homo sapiens
DIQMTQSPSSLSASVGDRVTITCRAGHNITNFLSWYQQKPGKAPTLLIYAVSNLQVGVPSRFSGSGSGAEFTLTISSLQPEDFATYYCQQNYNFSFTFGGGTKVDNK

SEQ ID NO: 292 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 294 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 295 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 296 - protein, 98 aa, Mus musculus
EVKLVESGGGLVKPGGSLKLSCAASGFTFSSYAMSWVRQTPEKRLEWVATISTGGGYTYFPDSVKGRFTISRDNAKNALYLQMKSLRSEDTAMYYCAR

SEQ ID NO: 297 - protein, 98 aa, Mus musculus
EVQLVESGGGLVKPGGSLKLSCAASGFTFSSYAMSWVRQTPEKRLEWVATISDGGSYTYYPDNVKGRFTISRDNAKNNLYLQMSHLKSEDTAMYYCAR

SEQ ID NO: 298 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 299 - protein, 98 aa, Homo sapiens
EVQLVESGGGLVKPGGSLKLSCAASGFTFSSYAMSWVRQTPEKRLEWVATISDGGGYTYYPDSVKGRFTISRDNAKNALYLQMKSLRSEDTAMYYCAR

SEQ ID NO: 300 - protein, 100 aa, Mus musculus
DVLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVP

SEQ ID NO: 301 - protein, 100 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVP

SEQ ID NO: 302 - protein, 100 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWP

SEQ ID NO: 303 - protein, 100 aa, Homo sapiens; identical to SEQ ID NO: 301

SEQ ID NO: 304 - protein, 448 aa, Homo sapiens
EVQLVESGGGLVQPGGSLILSCAASGFTFSSYAMTWVRQAPGKGLEWVCAISGSGSTYYADSVKGRFTCSRDNSKNTLYLQMNSLRAEDTAVYYCARRGLRYNYFDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 453 aa, Homo sapiens
EVQLVESGGGLVQPGGSLILSCAASGFTFTTYNMNWVRQAPGKGLEWVCVISTGGSNTYYADSVKGRFTCSRDNSKNTLYLQMNSLRAEDTAVYYCARDGGYSSSTLYAMDAWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 458 aa, Homo sapiens
EVQLVESGGGLVQPGGSLILSCAASGFTFSNHWMNWVRQAPGKGLEWVCAAINEDGSEKYYVGSVKVRFTCSRDNSKNTLYLQMNSLRAEDTAVYYCARDYYDILTDYYIHYWYFDLWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 447 aa, Homo sapiens
EVQLVESGGGLVQPGGSLILSCAASGYTFTHYGMNWVRQAPGKGLEWVCWINTYTGEPTYADDFKERFTCSRDNSKNTLYLQMNSLRAEDTAVYYCARERGDAMDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 451 aa, Homo sapiens
EVQLVESGGGLVQPGGSLILSCAASGYSITSGYSWNWVRQAPGKGLEWVCSITYDGSTNYADSVKGRFTCSRDNSKNTLYLQMNSLRAEDTAVYYCARGSHYFGHWHFAVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 449 aa, Homo sapiens
EVQLVESGGGLVQPGGSLILSCAASGFSLTNYGVHWVRQAPGKGLEWVCVIWSGGNTDYNTPFTSRFTCSRDNSKNTLYLQMNSLRAEDTAVYYCARALTYYDYEFAYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 452 aa, Homo sapiens
EVQLVESGGGLVQPGGSLILSCAASGFTFSSYAMSWVRQAPGKGLEWVCGITGSGGSTYYADSVKGRFTCSRDNSKNTLYLQMNSLRAEDTAVYYCARDPGTTVIMSWFDPWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 450 aa, Homo sapiens
EVQLVESGGGLVQPGGSLILSCAASGFNIKDTYIHWVRQAPGKGLEWVCRIYPTNGYTRYADSVKGRFTCSRDNSKNTLYLQMNSLRAEDTAVYYCARWGGDGFYAMDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 445 aa, Homo sapiens
EVQLVESGGGLVQPGGSLILSCAASGFNIKDTYIHWVRQAPGKGLEWVCRIDPAKINTKYDPKFQGRFTCSRDNSKNTLYLQMNSLRAEDTAVYYCARYPIFDYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 127 aa, Homo sapiens
DVVLTQSPLSLAVLLGQPASISCRASQSVSSSYLAWYQQRPGQSPRLLIYGASSRATGVPDRFSGSGSGTDFTLKISRVEAEDVGVYFCQQYGRSLEYTFGGGTKLEIKRTVAAPSVFIFPPSDEQL

SEQ ID NO: 314 - protein, 125 aa, Homo sapiens
DVVLTQSPLSLAVLLGQPASISCRASKNIYNALAWYQQRPGQSPRLLIYNTDRLHTGVPDRFSGSGSGTDFTLKISRVEAEDVGVYFCQHYFDYPRTFGGGTKLEIKRTVAAPSVFIFPPSDEQL

SEQ ID NO: 315 - protein, 126 aa, Homo sapiens
DVVLTQSPLSLAVLLGQPASISCRASQSVSSSYLAWYQQRPGQSPRLLIYGASSRATGVPDRFSGSGSGTDFTLKISRVEAEDVGVYFCQQYGSSPLTFGGGTKLEIKRTVAAPSVFIFPPSDEQL

SEQ ID NO: 316 - protein, 123 aa, Homo sapiens
DVVLTQSPLSLAVLLGQPASISCTASQSVSNDVVWYQQRPGQSPRLLIYSAFNRYTGVPDRFSGSGSGTDFTLKISRVEAEDVGVYFCQQDYNSPRTFGGGTKLEIKRTVAAPSVFIFPPSDE

SEQ ID NO: 317 - protein, 127 aa, Homo sapiens
DVVLTQSPLSLAVLLGQPASISCRASQSVDYDGDSYMNWYQQRPGQSPRLLIYAASYLESGVPDRFSGSGSGTDFTLKISRVEAEDVGVYFCQQSHEDPYTFGGGTKLEIKRTVAAPSVFIFPPSDE

SEQ ID NO: 318 - protein, 123 aa, Homo sapiens
DVVLTQSPLSLAVLLGQPASISCRASQSIGTNIHWYQQRPGQSPRLLIYYASESISGVPDRFSGSGSGTDFTLKISRVEAEDVGVYFCQQNNNWPTTFGGGTKLEIKRTVAAPSVFIFPPSDE

SEQ ID NO: 319 - protein, 124 aa, Homo sapiens
DVVLTQSPLSLAVLLGQPASISCRASQSVRGRYLAWYQQRPGQSPRLLIYGASSRATGVPDRFSGSGSGTDFTLKISRVEAEDVGVYFCQQYGSSPRTFGGGTKLEIKRTVAAPSVFIFPPSDE

SEQ ID NO: 320 - protein, 123 aa, Homo sapiens
DVVLTQSPLSLAVLLGQPASISCRASQDVNTAVAWYQQRPGQSPRLLIYSASFLYSGVPDRFSGSGSGTDFTLKISRVEAEDVGVYFCQQHYTTPPTFGGGTKLEIKRTVAAPSVFIFPPSDE

SEQ ID NO: 321 - protein, 123 aa, Homo sapiens
DVVLTQSPLSLAVLLGQPASISCSASQGIRNYLNWYQQRPGQSPRLLIYYTSSLHSGVPDRFSGSGSGTDFTLKISRVEAEDVGVYFCQQYSNFPWTFGGGTKLEIKRTVAAPSVFIFPPSDE

SEQ ID NO: 322 - protein, 450 aa, Homo sapiens
EVQLVESGGGLVQPGGSLILSCAASGFTFSSYAMSWVRQAPGKGLEWVCTISTGGGYTYFPDSVKGRFTCSRDNSKNTLYLQMNSLRAEDTAVYYCARQGDFGDWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 450 aa, Homo sapiens
EVQLVESGGGLVQPGGSLILSCAASGFTFSSYAMSWVRQAPGKGLEWVSTISTGGGYTYFPDSVKGRFTISRDNSKNTLYLQMNSLRAEDTAVYYCARQGDFGDWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 447 aa, Homo sapiens
EVQLVESGGGLVQPGGSLILSCAASGFNIKDTFMHWVRQAPGKGLEWVCRIDPANGNTEYDPKFQGRFTCSRDNSKNTLYLQMNSLRAEDTAVYYCARGGELGFPYWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 113 aa, Homo sapiens
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTKLEIKR

SEQ ID NO: 326 - protein, 113 aa, Homo sapiens
DVVLTQSPLSLAVLLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTKLEIKR

SEQ ID NO: 327 - protein, 108 aa, Homo sapiens
DVVLTQSPLSLAVLLGQPASISCRASGNIHNYLAWYQQRPGQSPRLLIYNAKTLADGVPDRFSGSGSGTDFTLKISRVEAEDVGVYFCQHFWSTPWTFGGGTKLEIKR

(19) United States
(12) Patent Application Publication
(10) Pub. No.: US 2014/0079691 A1
(43) Pub. Date: Mar. 20, 2014

(54) THERMOSTABLE ANTIBODY FRAMEWORK REGIONS

(71) Applicant: Anaptysbio, Inc., San Diego, CA (US)

(72) Inventors: Audrey MCCONNELL, San Diego, CA (US); David J. King, Encinitas, CA (US); Peter M. Bowers, San Diego, CA (US)

(21) Appl. No.: 14/031,782

(22) Filed: Sep. 19, 2013

Related U.S. Application Data
(60) Provisional application No. 61/703,569, filed on Sep. 20, 2012.

(51) Int. Cl. C07K 16/10

(52) U.S. Cl. CPC C07K 16/10
USPC 424/133.1; 530/387.3; 530/391.1; 536/23.53; 435/320.1; 514/44 R; 435/252.3; 435/252.31; 435/252.33; 435/252.35; 435/252.34; 435/254.2; 435/328; 435/254.23; 435/254.21

(57) ABSTRACT

The invention provides isolated amino acid sequences comprising the framework regions of an immunoglobulin heavy chain or light chain polypeptide, wherein certain amino acid residues of the framework regions are replaced with different amino acid residues that confer increased thermostability in vitro or in vivo. The invention also provides an isolated amino acid sequence of the constant region of an immunoglobulin heavy chain polypeptide wherein certain amino acid residues of the constant region are replaced with different amino acid residues that confer increased thermostability in vitro or in vivo.

### STATEMENT REGARDING FEDERALLY SPONSORED RESEARCH AND DEVELOPMENT

This invention was made with Government support under Contract Number N10PC20129 awarded by the Defense Advanced Research Projects Agency Antibody Technology Program. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 385,171 Byte ASCII (Text) file named “714198_ST25.TXT,” created on Sep. 18, 2013.

### BACKGROUND OF THE INVENTION

Antibodies are able to recognize a wide variety of antigens with extremely high specificity, making them ideal tools for a broad range of therapeutic, diagnostic, and industrial applications. Antibodies used for therapeutic purposes must have optimal pharmaceutical properties and desirably a long serum half-life, both of which are facilitated by thermal stability and resistance to aggregation (see, e.g., Willuda, et al., Cancer Res., 59: 5758-5767 (1999); and Carter et al., Curr. Opin. Biotechnol., 8: 449-454 (1997)). Antibodies used for industrial applications should retain their function following exposure to high temperatures, organic solvents, and other stresses not found in the in vivo environment (see, e.g., Dooley et al., Biotechnol. Appl. Biochem. 28(Pt 1): 77-83 (1998)). Antibody-based biosensors, for example, provide the most reliable detection capability across a broad range of targets, but require high stability and a long shelf life in order to be practically useful (see, e.g., Conroy et al., Semin. Cell. Dev. Biol., 20: 10-26 (2009)). Few antibodies, however currently possess these ideal biophysical properties. As such, recent research has focused on understanding and improving the stability of antibodies and antibody fragments (see, e.g., Caravella, et al., Curr. Comput. Aided. Drug. Des., Epublication in advance of print (Apr. 6, 2010); Ewert et al., J. Mol. Biol., 325: 531-53 (2003); Garber et al., Biochem. Biophys. Res. Commun., 355: 751-757 (2007); Jordan et al., Proteins, 77: 832-841 (2009); Monsellier et al., J. Mol. Biol., 362: 580-93 (2006); and Rothlisberger et al., J. Mol. Biol., 347: 773-789 (2005)).

Numerous knowledge-based, structure-based, and computational design-based approaches to engineering antibody stability have been described (see, e.g., Monsellier et al., J. Mol. Biol., 362: 580-593 (2006); and Worn et al., J. Mol. Biol., 305: 989-1010 (2001)). In addition, in silico approaches have been employed to engineer antibody-like molecules with enhanced thermal and chemical stability (see, e.g., Jordan et al., Proteins, 77: 832-41 (2009)), to reduce aggregation propensity of IgG constant domains (see, e.g., Chemamsetty et al., Proc. Natl. Acad. Sci. USA, 106: 11937-11942 (2009)), and to design antibodies with higher affinity for a given antigen (see, e.g., Farady et al., Bioorg. Med. Chem. Lett., 19: 3744-3747 (2009); and Clark et al., Protein Sci., 15: 949-960 (2006)). These thermostabilization methods, however, have yet to be employed in the more complex, full-length immunoglobulin context. In addition, each of these approaches involves the introduction of mutations that have the potential to disrupt antigen binding.

There remains a need for highly thermostable antibody framework amino acid sequences, as well as methods of generating such framework amino acid sequences. The invention provides such amino acids and methods.

### BRIEF SUMMARY OF THE INVENTION

The invention provides an isolated amino acid sequence which comprises the framework regions of an immunoglobulin heavy chain variable region polypeptide of any one of SEQ ID NO: 1-SEQ ID NO: 189, except that each of two or more of residues 5, 19, 49, 50, 51, 64, 68, 69, 70, 71, 72, 73, and 75 thereof is replaced with a different amino acid residue.

The invention also provides an isolated amino acid sequence which comprises the framework regions of an immunoglobulin light chain variable region polypeptide of any one of SEQ ID NO: 190-SEQ ID NO: 291, except that each of two or more of residues 4, 12, and 14 thereof is replaced with a different amino acid residue.

The invention provides an isolated amino acid sequence comprising the constant region of an immunoglobulin heavy chain polypeptide comprising of any one of SEQ ID NO: 292-SEQ ID NO: 295, except that each of residues 12 and 104 thereof is replaced with a different amino acid residue.

### BRIEF DESCRIPTION OF THE SEVERAL VIEWS OF THE DRAWINGS

FIG. 1 is a sequence alignment of (A) V_{H} and (B) V_{L} domains from a mouse anti-MS2 scFv as compared to the closest mouse and human germline variable regions. CDR1 and CDR2 sequences are shaded; CDR3 was excluded from the alignment. The scFv light chain is 99% germline.

FIGS. 2A-2D are graphs which depict experimental data illustrating the thermal unfolding and kinetic measurements of the CDR-grafted anti-MS2 IgG and the starting anti-MS2 scFv. FIG. 2A depicts data illustrating differential scanning calorimetry (DSC) analysis of the initial CDR-grafted antibody, APE443. FIG. 2B depicts data illustrating DSC analysis of the anti-MS2 scFv. Fitted peaks are shown with a dashed line and original thermograms are shown with a solid line. FIG. 2C depicts data illustrating that APE443 exhibited some loss in affinity for MS2, with a K_{H} of 170 nM (kₐ=6.8×10⁴ M⁻¹ s⁻¹, k_{d}=1.1×10⁻² s⁻¹). FIG. 2D depicts data from a Biacore sensogram showing that the anti-MS2 scFv had a K_{D} equal to 29 nM (kₐ=2.8×10⁵ M⁻¹ s⁻¹, k_{d}=8.0×10⁻³ s⁻¹). APE443 binds MS2 antigen with a K_{D} of 170 nM (Kₐ=6.8×10⁴M⁻¹ s⁻¹, k_{d}=1.1×10⁻² s⁻¹).

FIG. 3A is a diagram which depicts a method of increasing the thermostability of an antibody in accordance with the invention.

FIG. 3B is a structural model of the APE443 variable domain with the light chain in black and the heavy chain in gray. Key interface residues that were mutated back to the specificity donor sequence are indicated. FIG. 3C is a graph which depicts experimental data illustrating the improved stability of V_{H}/V_{L}-optimized APE556. FIG. 3D is a graph which depicts experimental data illustrating the restoration of wild-type affinity to APE556 (K_{D}=2 nM; kₐ=2.1×10⁵ M⁻¹ s⁻¹, k_{d}=5.5×10⁻³ s⁻¹).

FIG. 4A is a structural model of the APE556 antibody, in which the new disulfide bond connecting S49C and 169C is indicated. The structural model was generated using the RosettaDesign backrub application. FIG. 4B is a graph which depicts experimental data from DSC thermograms of progressively stabilized anti-MS2 antibody variants. Addition of the new disulfide bond to APE565 increased the Fab Tₘ by 5.9° C. relative to APE556. FIGS. 4C and 4D are graphs which depict experimental data illustrating a thermostability comparison of C_{H}2 variants. FIG. 4C displays the thermostability of the APE556 antibody variant, which exhibits a typical IgG1 C_{H}2 Tₘ of 69.4° C. by DSC. FIG. 4D displays the thermostability of the APE713 antibody variant, which shows that the addition of the C₁₂-C₁₀₄ disulfide bond increases the C_{H}2 Tₘ by 8.7° C.

FIG. 5A is a graph which depicts experimental data illustrating the progression of stabilization from the starting anti-MS2 scFv through the most stable construct, APE979.

FIG. 5B is a graph which depicts experimental data from a DSC thermogram of APE979, which demonstrates an increased Tₘ in histidine buffer, pH 7.0 (right panel), relative to PBS, pH 7.4 (left panel). The Fab and C_{H}2 domains were stabilized such that all three melting transitions overlapped under a single peak. FIG. 5C is a graph which depicts experimental data illustrating the antigen-binding activity of stabilized anti-MS2 antibody variants after a one-hour thermal challenge at the indicated temperature. Antigen binding was measured by Biacore T200. The scFv and APE443 antibody variants exhibited complete loss in antigen binding at the lowest temperature (70° C.), while stabilized APE979 retained greater than 60% activity after one hour at 89° C.

FIGS. 6A-6F are graphs which depict experimental data illustrating the MS2 affinity progression starting from the initial CDR-grafted antibody (APE443, FIG. 6A), and incorporating mutations from AID-induced SHM and library screening (APE1051-APE830, FIGS. 6B-6E) to arrive at the final mature antibody (APE850, FIG. 6F).

FIG. 7A is a structural model of the APE1027 Fab, in which the stabilizing mutations and affinity-improving mutations are indicated. FIG. 7B is a graph which depicts experimental data from DSC analysis of APE1027, which shows that stabilization is fully retained. FIG. 7C is a graph which depicts experimental data illustrating improved MS2 binding by the APE1027 antibody variant (K_{D}=880 pM, kₐ=9.7×10⁴ M⁻¹ s⁻¹, k_{d}=8.5×10⁻⁵ s⁻¹).

FIGS. 8A and 8B are graphs which depict experimental data illustrating the thermal unfolding and kinetic measurements of the anti-HA33 parental antibody and stabilized variants thereof. FIG. 8A depicts the three unfolding transitions, representing the Fab, CH2, and CH3 domains of the APE1136 starting Fab, the APE1148 chimeric IgG, and the APE1146 stable CDR-grafted IgG. FIG. 8B depicts a direct comparison of variable domain Tₘ values relative to the starting APE1136 anti-HA33 Fab.

FIG. 9A is a graph which depicts the results of a FACS analysis to determine IgG expression and binding affinity of the anti-HA33 antibodies described in Example 6. FIG. 9B is a graph which depicts experimental data illustrating the affects of various somatic hypermutation events on the antigen-binding affinity of the anti-HA33 antibodies described in Example 6. FIG. 9C is a graph which depicts experimental data illustrating the affinity of a mature anti-HA33 antibody containing all five enriching mutations produced by affinity maturation as described in Example 6.

FIG. 10 is a graph which depicts experimental data illustrating the Tₘ values for the stabilized therapeutically relevant antibodies described in Example 7.

FIGS. 11A-11E are graphs which depict experimental data comparing the Tₘ values for stabilized versions of Herceptin (FIG. 11A), Denosumab (FIG. 11B), an anti-TNFα antibody (FIG. 11C), Cetuximab (FIG. 11D), and Omalizumab (FIG. 11E) as compared to the parental versions of the antibodies.

FIG. 12 is a graph which depicts experimental data illustrating the change in Tₘ values for stable framework-CDR grafted versions of Denosumab, Herceptin, Omalizumab, Cetuximab, and an anti-TNFα antibody as compared to the parental version of the antibody.

FIG. 13A is a graph which depicts experimental data illustrating the change in Tₘ values for a stable framework-CDR grafted anti-ricin antibody as compared to the parental version of the antibody. FIG. 13B are graphs which depict experimental data illustrating the results of an ELISA assay showing that the stabilized anti-ricin antibody maintained full ricin binding activity after heating for 1 hour at 70° C., while the parental antibody lost all activity after heating for 40 minutes at 70° C.

### DETAILED DESCRIPTION OF THE INVENTION

The invention is predicated, at least in part, on a method of generating highly thermostable, high-affinity antibodies utilizing a combinatorial approach and in vitro affinity maturation. The invention provides an isolated amino acid sequence comprising the framework regions of an immunoglobulin heavy chain variable region polypeptide or the framework regions of an immunoglobulin light chain variable region polypeptide. This invention also provides an isolated amino acid sequence comprising a constant region of an immunoglobulin heavy chain variable region polypeptide. The term “immunoglobulin” or “antibody,” as used herein, refers to a protein that is found in blood or other bodily fluids of vertebrates, which is used by the immune system to identify and neutralize foreign objects, such as bacteria and viruses. In a preferred embodiment, an immunoglobulin or antibody is a protein that comprises at least one complementarity determining region, or CDR. The CDRs form the “hypervariable region” of an antibody, which is responsible for antigen binding (discussed further below). A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)). The amino acid sequences of numerous variable regions of human immunoglobulin heavy and light chain polypeptides, including the framework regions, have been identified and are publicly available from, for example, the National Center for Biotechnology's (NCBI) GenBank database. Examples of amino acid sequences of immunoglobulin heavy chain variable region polypeptides include SEQ ID NO: 1-SEQ ID NO: 189, while examples of amino acid sequences of immunoglobulin light chain variable region polypeptides include SEQ ID NO: 190-SEQ ID NO: 291.

The framework regions are connected by three complementarity determining regions (CDRs). As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, they can influence the orientation of the variable regions. The constant regions also exhibit various effector functions, such as participation in antibody-dependent cellular toxicity via interactions with effector molecules and cells.

The invention provides an isolated amino acid sequence which comprises the framework regions of an immunoglobulin heavy chain variable region polypeptide of any one of SEQ ID NO: 1-SEQ ID NO: 189, except that each of two or more residues within any one of the framework regions of SEQ ID NO: 1-SEQ ID NO: 189 is replaced with a different amino acid residue, i.e., an amino acid that differs from the native amino acid in that position. The replacement amino acid residue can be the same or different in each replacement position. For example, the amino acid residue of a first position can be replaced with a first different amino acid residue, and the amino acid residue of a second position can be replaced with a second different amino acid residue, wherein the first and second different amino acid residues are the same or different. The amino acid replacements can occur in any one of the four framework regions of SEQ ID NO: 1-SEQ ID NO: 189. In this respect, the amino acid replacements can occur in FR1, FR2, FR3, and/or FR4. Each of at least two amino acid residues within the framework regions of any one of SEQ ID NO: 1-SEQ ID NO: 189 is replaced with a different amino acid residue, but any number of amino acid residues of SEQ ID NO: 1-SEQ ID NO: 189 can be replaced with a different amino acid residue, so long as the amino acid replacements improve the stability of the inventive isolated amino acid sequence. Preferably, each of at least two amino acid residues (e.g., each of 3 or more, 5 or more, or 8 or more amino acid residues), but less than 20 amino acid residues (e.g., 18 or less, 15 or less, 12 or less, or 10 or less amino acid residues) of SEQ ID NO: 1-SEQ ID NO: 189 are replaced with a different amino acid residue. For example, each of as many as ten amino acid residues within the framework regions of any one of SEQ ID NO: 1-SEQ ID NO: 189 can be replaced with a different amino acid residue. In this respect, the isolated amino acid sequence can comprise the framework regions of an immunoglobulin heavy chain variable region polypeptide of any one of SEQ ID NO: 1-SEQ ID NO: 189, except that each of 2, 3, 4, 5, 6, 7, 8, 9, or 10 residues is replaced with a different amino acid residue.

The inventive isolated amino acid sequence comprises the framework regions of an immunoglobulin heavy chain variable region polypeptide of any one of SEQ ID NO: 1-SEQ ID NO: 189, except that each of two or more of residues 5, 19, 49, 50, 51, 64, 68, 69, 70, 71, 72, 73, and 75 of SEQ ID NO: 1-SEQ ID NO: 189 is replaced with a different amino acid residue. Each of amino acid residues 5, 19, 49, 50, 51, 64, 68, 69, 70, 71, 72, 73, and 75 of SEQ ID NO: 1-SEQ ID NO: 189 can be replaced with any suitable amino acid residue that can be the same or different in each position. An amino acid “replacement” or “substitution” refers to the replacement of one amino acid at a given position or residue by another amino acid at the same position or residue within a polypeptide sequence.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In one embodiment, the isolated amino acid sequence comprises the framework regions of an immunoglobulin heavy chain variable region polypeptide of any one of SEQ ID NO: 1-SEQ ID NO: 189, wherein (a) residue 5 of any one of SEQ ID NO: 1-SEQ ID NO: 189 is replaced with a valine (V) residue, (b) residue 19 of any one of SEQ ID NO: 1-SEQ ID NO: 189 is replaced with an isoleucine (I) residue, (c) residue 49 of any one of SEQ ID NO: 1-SEQ ID NO: 189 is replaced with a cysteine (C) residue, (d) residue 50 of any one of SEQ ID NO: 1-SEQ ID NO: 189 is replaced with a cysteine (C) residue, (e) residue 51 of any one of SEQ ID NO: 1-SEQ ID NO: 189 is replaced with a cysteine (C) residue, (f) residue 64 of any one of SEQ ID NO: 1-SEQ ID NO: 189 is replaced with a cysteine (C) residue, (g) residue 68 of any one of SEQ ID NO: 1-SEQ ID NO: 189 is replaced with a cysteine (C) residue, (h) residue 69 of any one of SEQ ID NO: 1-SEQ ID NO: 189 is replaced with a cysteine (C) residue, (i) residue 70 of any one of SEQ ID NO: 1-SEQ ID NO: 189 is replaced with a cysteine (C) residue, (j) residue 71 of any one of SEQ ID NO: 1-SEQ ID NO: 189 is replaced with a cysteine (C) residue, (k) residue 72 of any one of SEQ ID NO: 1-SEQ ID NO: 189 is replaced with a cysteine (C) residue, (l) residue 73 of any one of SEQ ID NO: 1-SEQ ID NO: 189 is replaced with a cysteine (C) residue, (m) residue 75 of any one of SEQ ID NO: 1-SEQ ID NO: 189 is replaced with a cysteine (C) residue, or any combination of two or more of the foregoing replacements.

In a preferred embodiment, the isolated amino acid sequence comprises the framework regions of an immunoglobulin heavy chain variable region polypeptide of any one of SEQ ID NO: 1-SEQ ID NO: 189, wherein (a) residue 5 of any one of SEQ ID NO: 1-SEQ ID NO: 189 is replaced with a valine (V) residue, (b) residue 19 of any one of SEQ ID NO: 1-SEQ ID NO: 189 is replaced with an isoleucine (I) residue, (c) residue 49 of any one of SEQ ID NO: 1-SEQ ID NO: 189 is replaced with a cysteine (C) residue, and (d) residue 69 of any one of SEQ ID NO: 1-SEQ ID NO: 189 is replaced with a cysteine (C) residue.

The invention also provides an isolated amino acid sequence which comprises the framework regions of an immunoglobulin light chain variable region polypeptide of any one of SEQ ID NO: 190-SEQ ID NO: 291, except that each of two or more residues within any one of the framework regions of SEQ ID NO: 190-SEQ ID NO: 291 is replaced with a different amino acid residue, i.e., an amino acid that differs from the native amino acid in that position. The replacement amino acid residue can be the same or different in each replacement position. For example, the amino acid residue of a first position can be replaced with a first different amino acid residue, and the amino acid residue of a second position can be replaced with a second different amino acid residue, wherein the first and second different amino acid residues are the same or different. The amino acid replacements can occur in any one of the four framework regions of SEQ ID NO: 190-SEQ ID NO: 291. In this respect, the amino acid replacements can occur in FR1, FR2, FR3, and/or FR4. At least two amino acid residues within the framework regions of any one of SEQ ID NO: 190-SEQ ID NO: 291 is replaced with a different amino acid residue, but any number of amino acid residues of SEQ ID NO: 190-SEQ ID NO: 291 can be replaced with a different amino acid residue, so long as the amino acid replacements improve the stability of the inventive isolated amino acid sequence. Preferably, each of at least two amino acid residues (e.g., each of 3 or more, 5 or more, or 8 or more amino acid residues), but less than 20 amino acid residues (e.g., 18 or less, 15 or less, 12 or less, or 10 or less amino acid residues), of SEQ ID NO: 190-SEQ ID NO: 291 are replaced with a different amino acid residue. For example, each of as many as ten amino acid residues within the framework regions of any one of SEQ ID NO: 190-SEQ ID NO: 291 can be replaced with a different amino acid residue. In this respect, the isolated amino acid sequence can comprise a framework region of an immunoglobulin light chain variable region polypeptide of any one of SEQ ID NO: 190-SEQ ID NO: 291, except that each of 2, 3, 4, 5, 6, 7, 8, 9, or 10 residues is replaced with a different amino acid residue.

The isolated amino acid sequence comprises the framework regions of an immunoglobulin light chain variable region polypeptide of any one of SEQ ID NO: 190-SEQ ID NO: 291, except that each of two or more of residues 4, 12, and 14 thereof is replaced with a different amino acid residue. In one embodiment, the isolated amino acid sequence comprises a framework region of an immunoglobulin light chain variable region polypeptide of any one of SEQ ID NO: 190-SEQ ID NO: 291, wherein (a) residue 4 of any one of SEQ ID NO: 190-SEQ ID NO: 291 is replaced with a leucine (L) residue, (b) residue 12 of any one of SEQ ID NO: 190-SEQ ID NO: 291 is replaced with an alanine (A) residue, (c) residue 14 of any one of SEQ ID NO: 190-SEQ ID NO: 291 is replaced with a leucine (L) residue, or any combination of two or more of the foregoing replacements.

In a preferred embodiment, the isolated amino acid sequence comprises a framework region of an immunoglobulin light chain variable region polypeptide of any one of SEQ ID NO: 190-SEQ ID NO: 291, wherein (a) residue 4 of any one of SEQ ID NO: 190-SEQ ID NO: 291 is replaced with a leucine (L) residue, (b) residue 12 of any one of SEQ ID NO: 190-SEQ ID NO: 291 is replaced with an alanine (A) residue, and (c) residue 14 of any one of SEQ ID NO: 190-SEQ ID NO: 291 is replaced with a leucine (L) residue.

The invention provides an isolated amino acid sequence comprising the constant region of an immunoglobulin heavy chain polypeptide comprising of any one of SEQ ID NO: 292-SEQ ID NO: 295, except that each of residues 12 and 104 of SEQ ID NO: 292-SEQ ID NO: 295 is replaced with a different amino acid residue, i.e., an amino acid that differs from the native amino acid in that position. The replacement amino acid residue can be the same or different in each replacement position. For example, the amino acid residue of a first position can be replaced with a first different amino acid residue, and the amino acid residue of a second position can be replaced with a second different amino acid residue, wherein the first and second different amino acid residues are the same or different. As discussed above, the constant region of an immunoglobulin heavy chain polypeptide is located at the C-terminus. The constant region determines the isotype, or class, of antibody, and is identical in all antibodies of the same isotype. The five major antibody isotypes are IgM, IgD, IgG, IgA, and IgE, and their heavy chains are denoted by the corresponding Greek letter (i.e., μ, δ, γ, α, and ε, respectively). Heavy chains γ, α, and δ have a constant region composed of three tandem Ig domains and a hinge region for added flexibility (see, e.g., Woof et al., Nat. Rev. Immunol., 4(2): 89-99 (2004)), while heavy chains μ and ε have a constant region composed of four immunoglobulin domains (see, e.g., Janeway et al., supra).

In one embodiment, the isolated amino acid sequence comprises the constant region of an immunoglobulin heavy chain polypeptide comprising any one of SEQ ID NO: 292-SEQ ID NO: 295, wherein (a) residue 12 is replaced with a cysteine (C) residue, or (b) residue 104 is replaced with a cysteine (C) residue. In a preferred embodiment, the isolated amino acid sequence comprises the constant region of an immunoglobulin heavy chain polypeptide comprising any one of SEQ ID NO: 292-SEQ ID NO: 295, wherein (a) residue 12 is replaced with a cysteine (C) residue, and (b) residue 104 is replaced with a cysteine (C) residue.

The invention provides an isolated antigen-binding agent comprising the inventive isolated amino acid sequences described herein. By “antigen-binding agent” is meant a molecule, preferably a proteinaceous molecule, that specifically binds to an antigen of interest. Preferably, the antigen-binding agent is an antibody or a fragment (e.g., immunogenic fragment) thereof. The isolated antigen-binding agent of the invention comprises the inventive isolated amino acid sequence comprising the framework regions of an immunoglobulin heavy chain variable region polypeptide, the inventive isolated amino acid sequence comprising the framework regions of an immunoglobulin light chain variable region polypeptide, and/or the inventive isolated amino acid sequence comprising the constant region of an immunoglobulin heavy chain polypeptide. In one embodiment, the isolated antigen-binding agent comprises the inventive amino acid sequences comprising the framework regions of an immunoglobulin heavy chain variable region polypeptide or the inventive amino acid sequence comprising the framework regions of an immunoglobulin light chain variable region polypeptide. In another embodiment, the isolated antigen-binding agent comprises the inventive amino acid sequence comprising the framework regions of an immunoglobulin heavy chain variable region polypeptide, the inventive amino acid sequence comprising the framework regions of an immunoglobulin light chain variable region polypeptide, and inventive amino acid sequence comprising the constant region of an immunoglobulin heavy chain polypeptide.

The invention is not limited to an isolated antigen-binding agent comprising an immunoglobulin heavy chain polypeptide or light chain polypeptide having replacements of the specific amino acid residues disclosed herein. Indeed, any amino acid residue of the framework regions of the inventive amino acid sequences encoding an immunoglobulin heavy chain variable region and/or light chain variable region, as well as any amino acid residue of the inventive amino acid sequence comprising the constant region of an immunoglobulin heavy chain polypeptide, can be replaced, in any combination, with a different amino acid residue, so long as the stability of the antigen-binding agent is enhanced or improved as a result of the amino acid replacements without concomitant loss of biological activity. The “biological activity” of an antigen-binding agent refers to, for example, binding affinity for a particular epitope, neutralization or inhibition of antigen activity in vivo (e.g., IC₅₀), pharmacokinetics, and cross-reactivity (e.g., with non-human homologs or orthologs of the antigen, or with other proteins or tissues). Other biological properties or characteristics of an antigen-binding agent recognized in the art include, for example, avidity, selectivity, solubility, folding, immunotoxicity, expression, formulation, and catalytic activity. The aforementioned properties or characteristics can be observed, measured, and/or assessed using standard techniques including, but not limited to, ELISA, competitive ELISA, BIACORE or KINEXA surface plasmon resonance analysis, in vitro or in vivo neutralization assays, receptor binding assays, cytokine or growth factor production and/or secretion assays, and signal transduction and immunohistochemistry assays. The stability of proteins such as immunoglobulins is discussed further herein.

The terms “inhibit” or “neutralize,” as used herein with respect to the activity of an antigen-binding agent, refer to the ability to substantially antagonize, prohibit, prevent, restrain, slow, disrupt, eliminate, stop, or reverse the progression or severity of, for example, the biological activity of an antigen, or a disease or condition associated with the antigen. The isolated antigen-binding agent of the invention preferably inhibits or neutralizes the activity of an antigen of interest by at least about 20%, about 30%, about 40%, about 50%, about 60%, about 70%, about 80%, about 90%, about 95%, about 100%, or a range defined by any two of the foregoing values.

The isolated antigen-binding agent of the invention can be a whole antibody, as described herein, or an antibody fragment. The terms “fragment of an antibody,” “antibody fragment,” or “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The isolated antigen-binding agent can contain any antigen-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, and (iii) a Fv fragment consisting of the V_{L} and V_{H} domains of a single arm of an antibody.

In embodiments where the isolated antigen-binding agent comprises a fragment of the immunoglobulin heavy chain or light chain polypeptide, the fragment can be of any size so long as the fragment binds to, and preferably inhibits the activity of, the antigen. In this respect, a fragment of the immunoglobulin heavy chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids. Similarly, a fragment of the immunoglobulin light chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids.

When the antigen-binding agent is an antibody or antibody fragment, the antibody or antibody fragment comprises a constant region (F_{c}) of any suitable class. Preferably, the antibody or antibody fragment comprises a constant region that is based upon wild type IgG1, IgG2, or IgG4 antibodies, or variants thereof.

The antigen-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

The isolated antigen-binding agent also can be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The isolated antigen-binding agent can be, or can be obtained from, a human antibody, a non-human antibody, or a chimeric antibody. By “chimeric” is meant an antibody or fragment thereof comprising both human and non-human regions. Non-human antibodies include antibodies isolated from any non-human animal, such as, for example, a rodent (e.g., a mouse or rat). While the inventive amino acid sequences comprise the framework regions of human heavy or light chain polypeptides, the inventive antigen-binding agent can comprise regions from a non-human antibody. For example, the inventive antigen-binding agent can comprise (1) a heavy chain polypeptide comprising the inventive amino acid sequence, (2) a light chain polypeptide comprising the inventive amino acid sequence, and (3) one or more CDRs obtained from a non-human antibody. In another embodiment, the inventive antigen-binding agent can comprise (1) a heavy chain polypeptide comprising the inventive amino acid sequence, (2) a light chain polypeptide obtained from a non-human antibody, and (3) one or more CDRs obtained from a non-human antibody. In another embodiment, the inventive antigen-binding agent can comprise (1) a heavy chain polypeptide obtained from a non-human antibody, (2) a light chain polypeptide comprising the inventive amino acid sequence, and (3) one or more CDRs obtained from a non-human antibody. These scenarios may be useful, e.g., for the humanization of an antibody.

A human antibody, a non-human antibody, or a chimeric antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)).

The invention also provides one or more isolated nucleic acid sequences that encode the aforementioned isolated amino acid sequences comprising the framework regions of an immunoglobulin heavy chain polypeptide, the framework regions of an immunoglobulin light chain polypeptide, and/or a constant region of an immunoglobulin heavy chain polypeptide, as well as one or more isolated nucleic acid sequences that encode the aforementioned inventive isolated antigen-binding agent.

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The invention further provides a vector comprising (a) a nucleic acid sequence encoding an inventive isolated amino acid sequence comprising the framework regions of an immunoglobulin heavy chain polypeptide, the framework regions of an immunoglobulin light chain polypeptide, and/or a constant region of an immunoglobulin heavy chain polypeptide, or (b) one or more nucleic acid sequences encoding the inventive antigen-binding agent. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

In addition to the nucleic acid sequence encoding the inventive amino acid sequences or the inventive antigen-binding agent, the vector preferably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150:1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.), and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXI™ from Promega (Madison, Wis.).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, Calif.), and the retroviral vectors pFB-ERV plus pCFB-EGSH from Stratagene (La Jolla, Calif.).

Nucleic acid sequences encoding the inventive amino acid sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously or sequentially.

The vector(s) comprising the nucleic acid(s) encoding the inventive amino acid sequences can be introduced into a host cell that is capable of expressing the polypeptides encoded thereby, including any suitable prokaryotic or eukaryotic cell. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

Preferably, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, Calif.).

Preferably, mammalian cells are utilized in the invention. A number of suitable mammalian host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

Most preferably, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS(CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

A nucleic acid sequence encoding the inventive amino acid sequence may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell. Biol., 7: 2031-2034 (1987)). Phage or viral vectors can be introduced into host cells, after growth of infectious particles in suitable packaging cells, many of which are commercially available.

The invention provides a composition comprising the inventive isolated amino acid sequences, the inventive antigen-binding agent, or the inventive vector comprising a nucleic acid sequence encoding any of the foregoing. Preferably, the composition is a pharmaceutically acceptable (e.g., physiologically acceptable) composition, which comprises a carrier, preferably a pharmaceutically acceptable (e.g., physiologically acceptable) carrier, and the inventive amino acid sequences, antigen-binding agent, or vector. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

The invention provides a method of improving the antigen-binding activity of the inventive isolated amino acid sequences described herein, as well as the inventive isolated antigen-binding agent described herein, which comprises subjecting a nucleic acid sequence encoding the inventive amino acid sequence or the inventive antigen-binding agent to somatic hypermutation (SHM). As used herein, “somatic hypermutation” or “SHM” refers to the mutation of a polynucleotide sequence which can be initiated by, or associated with, the action of activation-induced cytidine deaminase (AID), which includes members of the AID/APOBEC family of RNA/DNA editing cytidine deaminases that are capable of mediating the deamination of cytosine to uracil within a DNA sequence (see, e.g., Conticello et al., Mol. Biol. Evol., 22: 367-377 (2005), and U.S. Pat. No. 6,815,194). SHM can also be initiated by, or associated with the action of, e.g., uracil glycosylase and/or error prone polymerases on a polynucleotide sequence of interest. SHM is intended to include mutagenesis that occurs as a consequence of the error prone repair of an initial DNA lesion, including mutagenesis mediated by the mismatch repair machinery and related enzymes.

In certain embodiments of the invention, AID can be endogenous to the cells described herein which express the inventive amino acid sequences. Alternatively, a nucleic acid encoding AID may be provided to cells which do, or which do not, contain an endogenous AID protein. The exogenously provided AID can be a wild-type AID, which refers to a naturally occurring amino acid sequence of an AID protein. Suitable wild-type AID proteins include all vertebrate forms of AID, including, for example, primate, rodent, avian, and bony fish. Representative examples of wild-type AID amino acid sequences are disclosed in, for example, U.S. Pat. Nos. 6,815,194; 7,083,966; and 7,314,621, and International Patent Application Publication WO 2010/113039. The use of AID in SHM systems is described in detail in, for example, U.S. Patent Application Publication 2009/0075378 A1 and International Patent Application Publications WO 2008/103474 and WO 2008/103475.

In other embodiments, the exogenously provided AID can be an “AID mutant” or a “mutant of AID.” As used herein, an “AID mutant” or a “mutant of AID” refers to an AID amino acid sequence that differs from a wild-type AID amino acid sequence by at least one amino acid. Preferably, an AID mutant is a “functional mutant of AID” or a “functional AID mutant,” which refers to a mutant AID protein which retains all or part of the biological activity of a wild-type AID, or which exhibits increased biological activity as compared to a wild-type AID protein. Suitable mutant AID proteins which exhibit increased biological activity as compared to a wild-type AID protein are described in, for example, Wang et al., Nat. Struct. Mol. Biol., 16(7): 769-76 (2009), and International Patent Application Publication WO 2010/113039.

In still other embodiments, SHM can be initiated by, or associated with the action of, an “AID homolog.” The term “AID homolog” refers to the enzymes of the Apobec family and include, for example, Apobec-1, Apobec3C, or Apobec3G (described, for example, in Jarmuz et al., Genomics, 79: 285-296 (2002)). The term “AID activity” includes activity mediated by AID and AID homologs.

There are a variety of nucleic acid sequences, such as genetic elements, that one of ordinary skill in the art would prefer to not undergo SHM in order to maintain overall system integrity. Examples of such nucleic acid sequences include (i) selectable markers, (ii) reporter genes, (iii) genetic regulatory signals, (iv) enzymes or accessory factors used for high level enhanced SHM, or its regulation or measurement (e.g., AID or a functional AID mutant, pol eta, transcription factors, and MSH2), (v) signal transduction components (e.g., kinases, receptors, and transcription factors), and (vi) domains or sub domains of proteins (e.g., nuclear localization signals, transmembrane domains, catalytic domains, protein-protein interaction domains, and other protein family conserved motifs, domains, and sub-domains).

The invention also provides a method of improving the antigen-binding activity of the inventive amino acid sequences, as well as the inventive isolated antigen-binding agent, which comprises deleting 1-10 amino acid residues (e.g., 1, 2, 3, 4, 5, 6, 7, 8, 9, or 10 residues, or a range defined by any two of the foregoing values) from the inventive amino acid sequence or the inventive isolated antigen-binding agent. The deletion of one or more amino acid residues can occur as a result of a deletion mutation introduced into a nucleic acid sequence encoding the inventive amino acid sequence or the inventive isolated antigen-binding agent. The term “deletion mutation,” as used herein, refers to the removal or loss of one or more nucleotides from a nucleic acid sequence, and is also referred to in the art as a “gene deletion,” a “deficiency,” or a “deletion.” A deletion mutation can be introduced at any suitable location of the nucleic acid sequence encoding the inventive amino acid sequences. For example, a deletion mutation can be introduced into the region of the nucleic acid sequence that encodes the variable region or the constant region of an immunoglobulin heavy or light chain. Preferably, the deletion mutation is introduced into the region of the nucleic acid sequence that encodes the variable region of an immunoglobulin heavy or light chain polypeptide.

The aforementioned amino acid replacements can occur by any suitable method known in the art, but preferably are generated using methods for improving the stability of the inventive isolated amino acid sequence in vitro and/or in vivo. The term “stability,” as used herein, refers to the ability of a protein to retain its structural conformation and/or its activity when subjected to physical and/or chemical manipulations. Such physical and/or chemical manipulations include, for example, exposure to high or low temperatures (i.e., thermostability), exposure to organic solvents, immunoglobulin aggregation, and other stresses not normally present in the in vivo environment (see, e.g., Willuda et al., supra, Carter et al., supra, and Dooley et al., supra).

In one embodiment, the amino acid replacements are generated using a method (or combination of methods) for improving the thermostability of the amino acid sequence in vitro and/or in vivo. Any suitable method for improving protein or antibody thermostability can be used in the context of the invention. One example of such a method is CDR grafting. Grafting CDRs of defined specificity onto known stable framework regions has been demonstrated to improve antibody stability (see, e.g., Jung et al., Protein Eng., 10: 959-966 (1997); and Jung et al., J. Mol. Biol., 294: 163-180 (1999)). However, CDR grafting frequently results in a loss of antigen-binding affinity, especially when CDRs are grafted into distantly related framework regions (see, e.g., Jones et al., Nature, 321: 522-525 (1986); Queen et al., Proc. Natl. Acad. Sci. USA, 86: 10029-10033 (1989); and Honegger et al., Protein Eng. Des. Sel., 22: 121-134 (2009)), resulting in the need for extensive affinity maturation to restore antigen binding activity.

Another method for improving antibody stability is consensus design, which utilizes the natural variation present within antibody variable domain sequences to identify non-canonical residues within a candidate antibody. The introduction of consensus residues into structurally equivalent positions in a candidate antibody has been demonstrated to improve the stability of immunoglobulin (IgG) variable regions (see, e.g., Steipe et al., J. Mol. Biol., 240: 188-192 (1994); and Chowdhury et al., J. Mol. Biol., 281: 917-928 (1998)), and has been applied more broadly to stabilize non-IgG proteins (see, e.g., Steipe, B., Methods Enzymol., 388: 176-186 (2004)). Introducing non-native disulfide bonds is another method for stabilizing proteins (see, e.g., Matsumura et al., Proc. Natl. Acad. Sci. USA, 86: 6562-6566 (1989); and Trivedi et al., Curr. Protein Pept. Sci., 10: 614-625 (2009)), inasmuch as native antibodies achieve much of their intrinsic stability through highly conserved intra-domain disulfide bonds which occur in folded domains (see, e.g., Frisch et al., Fold. Des., 1: 431-440 (1996); and Goto et al., J. Biochem., 86: 1433-1441 (1979)). Inter-domain disulfide bonds have been introduced between V_{H} and V_{L} domains to enhance the stability of single-chain Fv (scFv) antibody fragments (see, e.g., Reiter et al., Protein Eng., 7: 697-704 (1994)); and Young et al., FEBS Lett., 377: 135-139 (1995)). In one embodiment, the amino acid replacements in the inventive isolated amino acid sequence are generated by introducing an intra-domain disulfide bond that has been identified within a Camelidae-derived V_{HH} antibody fragment (see Saerens et al., J. Mol. Biol., 377: 478-488 (2008)). This Camelidae intra-domain disulfide bond has been shown to provide additional stability when transferred onto other V_{HH} antibodies without adversely affecting antigen binding (Saerens et al., supra). In addition, a disulfide bond linking N- and C-terminal β-strands of an isolated C_{H}2 constant domain has been shown to significantly increase its stability in both human and mouse (see, e.g., Gong et al., J. Biol. Chem., 284: 14203-14210 (2009)), and can be used in the context of the invention.

In addition to the knowledge-based methods for improving protein stability described above, structure-based, computational design methods for the stabilization of proteins can be used to introduce the amino acid replacements in the inventive isolated amino acid sequences. Such structure-based, computational design methods have been used in the de novo redesign of natural protein domains (described in, e.g., Dahiyat et al., Science, 278: 82-87 (1997)), the thermodynamic stabilization of natural protein domains (described in, e.g., Dantas et al. J. Mol. Biol., 332: 449-460 (2003)), and the creation of extremely stable novel protein structures (described in, e.g., Dantas et al., J. Mol., Biol., 366: 1209-1221 (2007); and Kuhlman et al., Science, 302: 1364-1368 (2003)). In silico approaches also have been used in the art to engineer antibody-like molecules with enhanced thermal and chemical stability (see, e.g., Jordan et al., Proteins, 77: 932-841 (2009)), to reduce the aggregation propensity of IgG constant domains (see, e.g., Chemamsetty et al., Proc. Natl. Acad. Sci. USA, 106: 11937-11942 (2009)), and to design antibodies with higher affinity for a given antigen (see, e.g., Farady et al., Bioorg. Med. Chem. Lett., 19: 3744-3747 (2009); and Clark et al., Protein Sci., 15: 949-960 (2006)).

In the context of the invention, stability of the inventive isolated amino acid sequences can be measured using any suitable assay known in the art, such as, for example, measuring serum half-life, differential scanning calorimetry, thermal shift assays, and pulse-chase assays. Other methods of measuring protein stability in vivo and in vitro that can be used in the context of the invention are described in, for example, Protein Stability and Folding, B. A. Shirley (ed.), Human Press, Totowa, N.J. (1995); Protein Structure, Stability, and Interactions (Methods in Molecular Biology), Shiver J. W. (ed.), Humana Press, New York, N.Y. (2010); and Ignatova, Microb. Cell Fact., 4: 23 (2005).

The stability of the inventive amino acid sequences can be measured in terms of the transition mid-point value (Tₘ), which is the temperature where 50% of the amino acid sequence is in its native confirmation, and the other 50% is denatured. In general, the higher the Tₘ, the more stable the protein. In one embodiment of the invention, the inventive isolated amino acid sequences comprise a transition mid-point value (Tₘ) in vitro of about 70-100° C. For example, the inventive isolated amino acid sequences can comprise a Tₘ in vitro of about 70-80° C. (e.g., 71° C., 75° C., or 79° C.), about 80-90° C. (e.g., about 81° C., 85° C., or 89° C.), or about 90-100° C. (e.g., about 91° C., about 95° C., or about 99° C.).

The amino acid replacements in the inventive isolated amino acid sequence can be generated using any one of the above-described methods for improving protein (e.g., antibody stability). Preferably, however, the amino acid replacements in the inventive isolated amino acid sequence are generated using a combination of the above-described methods for improving protein stability. In this respect, the invention provides a method of producing the inventive isolated amino acid sequence which comprises the framework regions of an immunoglobulin heavy chain variable region polypeptide, which method comprises providing an amino acid sequence which comprises an unmodified framework region of an immunoglobulin heavy chain variable region, and subjecting the amino acid sequence to one or more of the following: (a) grafting one or more non-native complementarity determining regions (CDR) into the amino acid sequence, (b) introducing one or more non-native disulfide bonds into the amino acid sequence, (c) introducing one or more non-native consensus amino acid residues into the amino acid sequence, or (d) introducing one or more stabilizing amino acid residues into the amino acid sequence, whereby a thermostable framework region of an immunoglobulin heavy chain variable region is produced. The invention also provides a method of producing the inventive isolated amino acid sequence which comprises a framework region of an immunoglobulin light chain variable region polypeptide, which method comprises providing an amino acid sequence which comprises an unmodified framework region of an immunoglobulin light chain variable region, and subjecting the amino acid sequence to one or more of the following: (a) grafting one or more non-native complementarity determining regions (CDR) into the amino acid sequence, (b) introducing one or more non-native disulfide bonds into the amino acid sequence, (c) introducing one or more non-native consensus amino acid residues into the amino acid sequence, or (d) introducing one or more stabilizing amino acid residues into the amino acid sequence, whereby a thermostable framework region of an immunoglobulin light chain variable region is produced. The invention further comprises a method of producing the inventive isolated amino acid sequence which comprises a constant region of an immunoglobulin heavy chain polypeptide, which method comprises (a) grafting one or more non-native complementarity determining regions (CDR) into the amino acid sequence, (b) introducing one or more non-native disulfide bonds into the amino acid sequence, (c) introducing one or more non-native consensus amino acid residues into the amino acid sequence, or (d) introducing one or more stabilizing amino acid residues into the amino acid sequence

In a preferred embodiment, the inventive amino acid sequences are produced by providing an amino acid sequence comprising an unmodified framework region of an immunoglobulin heavy or light chain variable region and (a) grafting one or more non-native complementarity determining regions (CDR) into the amino acid sequence, (b) introducing one or more non-native disulfide bonds into the amino acid sequence, (c) introducing one or more non-native consensus amino acid residues into the amino acid sequence, and (d) introducing one or more stabilizing amino acid residues into the amino acid sequence.

In one embodiment, the aforementioned method of producing the inventive amino acid sequences further comprises subjecting a nucleic acid sequence encoding the thermostable framework region of an immunoglobulin heavy chain variable region, an immunoglobulin light chain region, or a constant region of a heavy chain polypeptide to somatic hypermutation (SHM). It is believed that subjecting thermostable framework regions, such as those described herein, to affinity maturation restores or improves the antigen binding-activity of an immunoglobulin heavy or light chain polypeptide that can be lost as a result of protein stabilization methods. The various aspects of SHM described above in connection with the aforementioned inventive amino acid sequences also apply to the aforementioned inventive method.

Following affinity maturation of the inventive amino acid sequences, a particular immunoglobulin heavy chain polypeptide or light chain polypeptide having a desired antigen affinity can be selected using any one of a variety of methods known in the art. For example, display technologies such as phage, yeast, and ribosome display can be used in the invention. Such display technologies are based on the in vitro selection of antibody fragments from libraries and overcome limitations of immune tolerance or epitope dominance in vivo (see, e.g., Hoogenboom, Nat. Biotech., 23: 1105-1116 (2005)). In a preferred embodiment, mammalian display technologies are used in the context of the invention to select an appropriate immunoglobulin heavy and/or light chain polypeptide. Mammalian cell expression systems offer several potential advantages for antibody generation (e.g., therapeutic antibodies) including the ability to co-select for key manufacturing properties such as high-level expression and stability, while displaying functional glycosylated IgGs on the cell surface. Mammalian cell display methods are further described in, e.g., Lanzavecchia et al., Curr. Opin. Biot., 18(6): 523-528 (2007); Beerli et al., Proc. Natl. Acad. Sci. USA, 105(38): 14336-14341 (2008); Kwakkenbos et al., Nat. Med., 16: 123-128 (2010); Zhou et al., mAbs, 2(5): 508-518 (2010); and Bowers et al., Proc. Natl. Acad. Sci. USA, 108(51): 20455-20460 (2011)).

The following examples further illustrate the invention but, of course, should not be construed as in any way limiting its scope.

### EXAMPLE 1

This example demonstrates a method of grafting CDRs from a mouse antibody onto a stable human framework region.

With the goal of generating a broadly useful IgG scaffold, stable basis V_{H} and V_{L} domains were selected as a starting point for CDR grafting. Previous studies have demonstrated that V_{H}3 is the most stable family of human V_{H} domains (Ewert et al., J. Mol. Biol., 325: 531-553 (2003)), and that V_{H}3-23 is one of the most commonly utilized human germline heavy chain variable regions (Glanville et al., Proc. Natl. Acad. Sci. USA, 106: 10216-20221 (2009)). Although there is less variation among the V_{L} domains, Vκ1, Vκ2, and Vκ3 domains are among the most stable of the eight human V_{L} domain subgroups (Ewert et al., supra).

An alignment of a mouse single-chain Fv (scFv) fragment targeting the MS2 bacteriophage coat protein (anti-MS2 scFv) with known human V_{H} domains and V_{L} domains showed that the human V_{H} and V_{L} regions with highest homology to the anti-MS2 scFv were the V_{H}3 and Vκ2 families. The anti-MS2 scFv was produced by panning a library generated from mice immunized with MS2 phage, and was obtained from the U.S. Army's Edgewood Chemical Biological Center (ECBC) as part of the Defense Advance Research Projects Agency (DARPA) Antibody Technology Program. Originally isolated as a Fab, the antibody fragment was converted to the scFv format with a (Gly4Ser)₃ linker between V_{H} and V_{L} domains, expressed transiently in HEK293 c-18 cells, and purified using standard his-tag affinity purification methodologies.

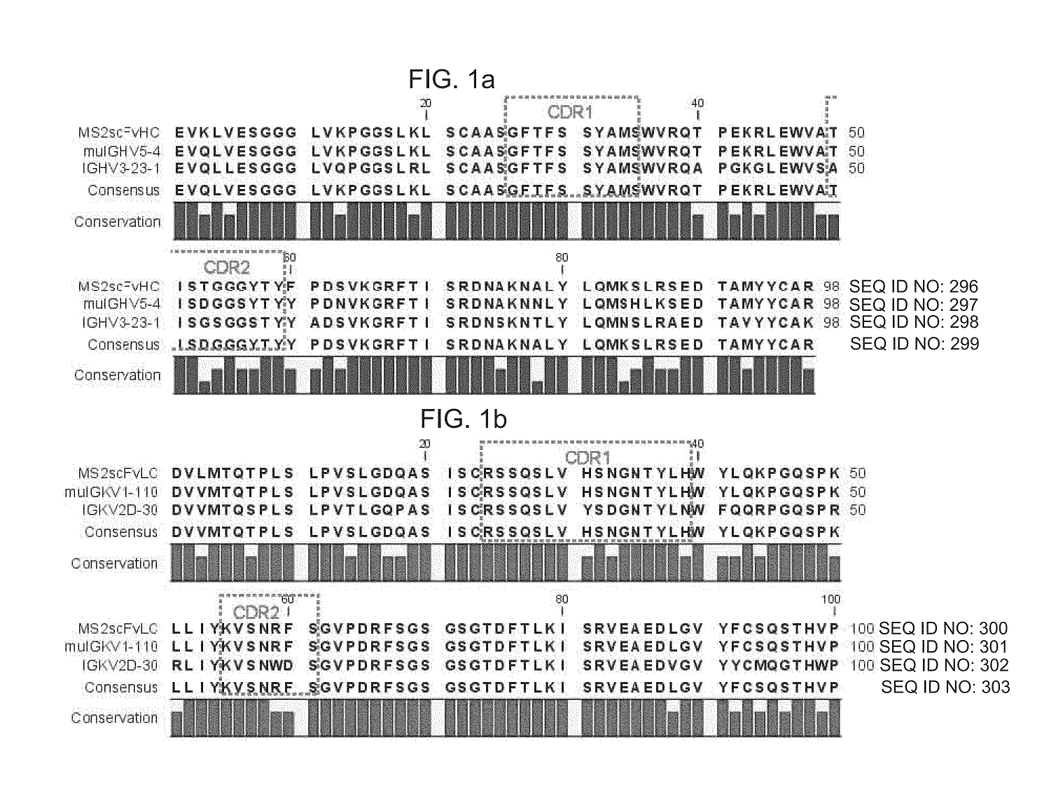

Based on the alignment, which is illustrated in FIGS. 1A and 1B, human germline variable regions hV_{H}3-23 and hVκ2D-30, each of which share 80% amino acid identity to the scFv, excluding the CDR3, were selected as the starting point for grafting and stabilization. CDR1, CDR2 and CDR3 of the mouse anti-MS2 scFv were grafted into the hV_{H}3-23 and hVκ2D-30 frameworks, and were formatted as full-length immunoglobulin, denoted APE443, using the stable human constant regions IgG1z kappa (Garber et al., Biochem. Biophys. Res. Commun., 355:751-757 (2007); and Demarest et al., J. Mol. Biol., 335: 41-48 (2004)).

Full-length human IgG antibody variants also were expressed transiently in HEK293 c-18 cells, purified using a protein A/G agarose resin (Thermo Scientific, Waltham, Mass.), washed with 6 column-volumes of 1×PBS, pH 7.4, and eluted with 100 mM glycine, pH 3.0, followed by buffer exchange into 1×PBS, pH 7.4. All mutagenesis was carried out using the QuickChange II site-directed mutagenesis kit (Agilent Technologies, Santa Clara, Calif.).

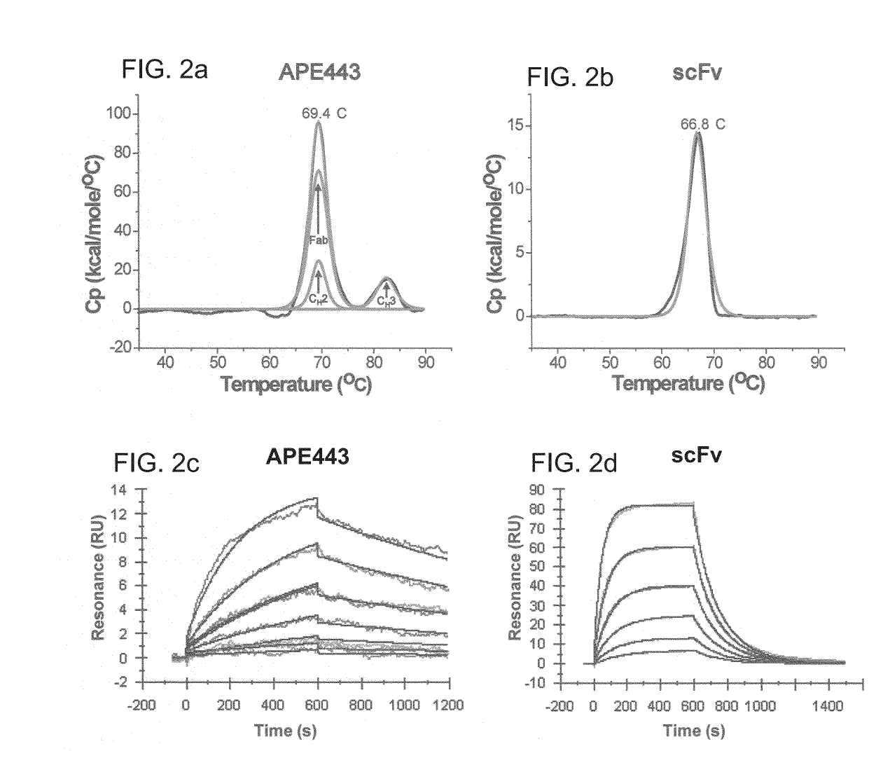

The thermal unfolding profiles of the anti-MS2 antibody variants were measured by differential scanning calorimetry (DSC) using the VP-Capillary DSC system (GE Healthcare, Waukesha, Wis.). All antibodies were tested in phosphate buffered saline (PBS), pH 7.4 at protein concentrations ranging from 0.7-1.0 mg/ml at a scan rate of 1° C./minute. DSC analysis of the initial grafted anti-MS2 scFv exhibited a typical IgG curve with three unfolding transitions, as shown in FIG. 2A. A direct comparison of variable domains revealed a modest 2.6° C. improvement in transition mid-point values (Tₘ) relative to the starting anti-MS2 scFv, as shown in FIGS. 2A and 2B.

Binding affinity measurements for the antibody variants were obtained by surface immobilization of antigen. Specifically, kinetic analysis was performed using a Biacore T200 (GE Healthcare, Waukesha, Wis.). For scFv fragments, approximately 200 response units (RU) of MS2 were immobilized (CM5 chip), and samples were tested in a concentration range from approximately 10-fold above the K_{D} to approximately 10-fold below the K_{D}. For full-length IgGs, a capture assay was used to allow accurate assessment of antibody affinity. Antibody was captured on a surface of approximately 3,000 RU of mouse anti-human IgG Fc, and antigen was flowed over the captured IgG surface again using a range from 10-fold above to 10-fold below the K_{D} in each case. Surfaces were regenerated using 3 M MgCl₂. Association and dissociation kinetic values (kₐ and k_{d}) were determined from a best fit of the data with the 1:1 Langmuir global fitting procedure to sensorgrams using the Biacore T200 Evaluation Software, version 1.0.

Although the K_{D} values were not directly comparable, the kinetic analysis suggested that grafting MS2 CDRs into the human V-regions resulted in a modest loss of MS2 antigen-binding affinity relative to the originating scFv, with K_{D} values of 84 and 29 nM, respectively, as shown in FIGS. 2C and 2D.

The results of this example confirm the production of an amino acid sequence comprising a stable human framework region in accordance with the invention.

### EXAMPLE 2

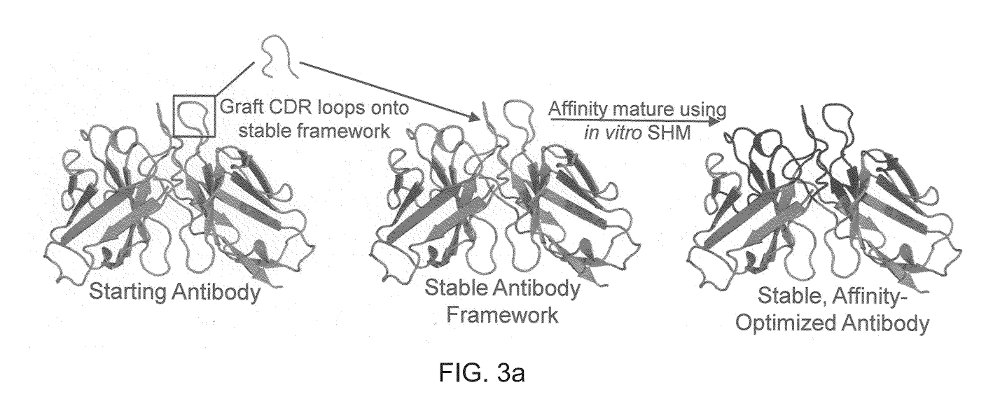

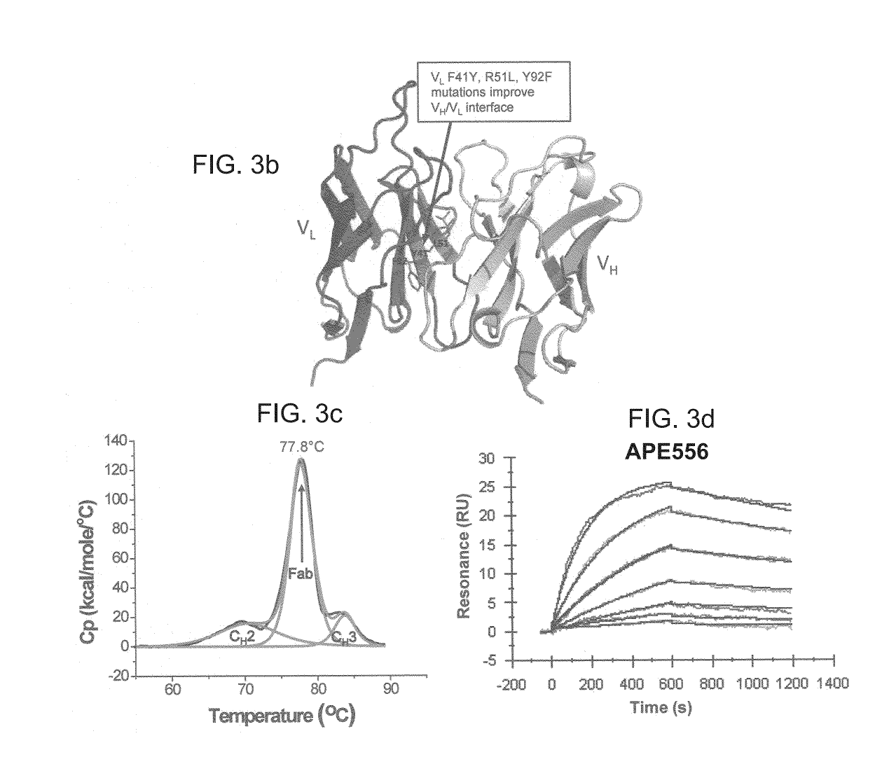

This example demonstrates methods of increasing the thermostability of a scFv antibody fragment. The overall strategy for improving antibody stability and affinity is depicted in FIG. 3A.

### Improving the V_{H}/V_{L} Heterodimer Interface

The interface between heavy and light chain variable domains can significantly impact both the stability and affinity of an antibody (Ewert et al., supra). Three interface residues were identified in the V_{L} that differed between the specificity donor (i.e., the anti-MS2 scFv described in Example 1) and acceptor (hVκ2D-30, described in Example 1) using the method outlined in Ewert et al., supra. Residues F36Y, R46L, and Y87F, were changed back to the original scFv sequence, as shown in FIG. 3B, and this modified anti-MS2 scFv was denoted APE556.

The thermal unfolding profile of APE556 was measured using differential scanning calorimetry (DSC) using the VP-Capillary DSC system (GE Healthcare). Antibodies were tested in phosphate buffered saline (PBS), pH 7.4, at protein concentrations ranging from 0.7-1.0 mg/ml at a scan rate of 1° C./minute. Samples were heated from 20-90° C. or 20-110° C. Data analysis was performed using Origin 7 software (OriginLab, Northampton, Mass.). Transition mid-point values (Tₘ) were determined from the thermogram data using the non-two-state model which employs the Levenberg-Marquardt non-linear least-square method. Total calorimetric heat change values (AH) were determined by calculating the total area under a given antibody thermogram.

DSC analysis of the APE556 scFv showed an 8.4° C. increase in Fab Tₘ over the initial graft, as illustrated in FIG. 3C. Improvement of the V_{H}/V_{L} interface restored MS2 binding affinity back to the wild-type K_{D}, as illustrated in FIG. 3D.

### Disulfide Bond Engineering—V_{H} Stabilization

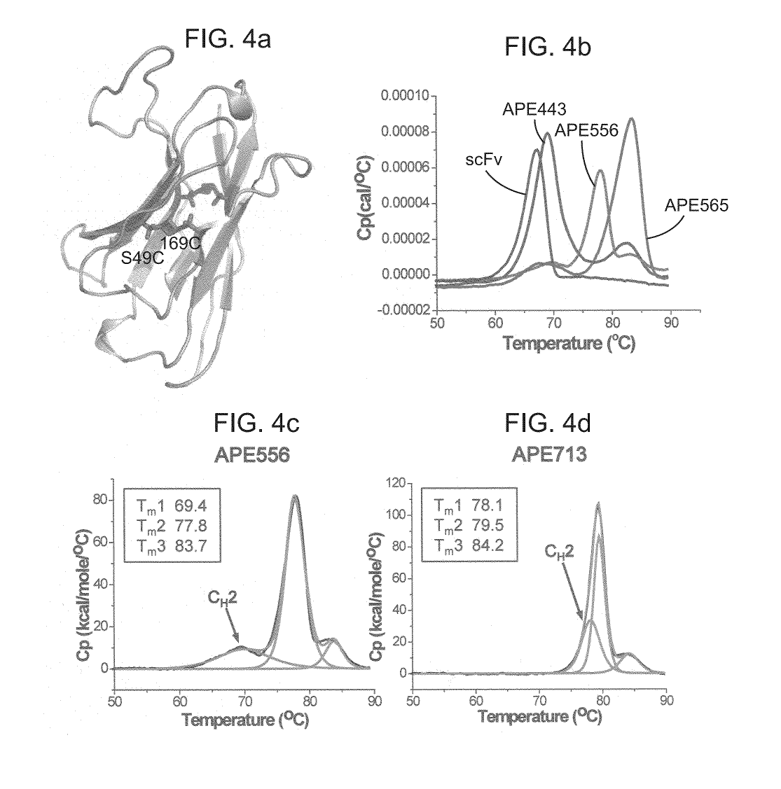

A naturally occurring, nonconserved disulfide bond within a single-domain antibody fragment derived from a Camelidae-specific heavy chain antibody (V_{HH}) has been shown to have a stabilizing effect when transferred to other V_{HH} fragments (Saerens et al., supra). To determine whether such a disulfide bond could be similarly stabilizing in the human IgG context, the amino acid sequence of this single-domain antibody was aligned with the MS2 antibody V_{H}. Homologous positions S49 and 169 were identified as candidates for disulfide bond insertion. These framework residues occur on opposing β-strands, similar to the conserved intra-V_{H} disulfide bond that connects framework residues C22 and C92, and is buried within the hydrophobic core of the V-region fold, as illustrated in FIG. 4A.

Computational methods were employed to assess whether the residues at positions 49 and 69 of APE556 (described above) were likely to accommodate a disulfide bond. The Disulfide by Design algorithm confirmed the appropriate geometry for intra-domain disulfide bond formation between residues S49 and 169 (see Dombkowski, A. A., Bioinformatics, 19: 1852-1853 (2003)). Furthermore, RosettaDesign (Rosetta Design Group, LLC, University of Washington) predicted cysteine (C) substitutions at these positions would form a disulfide bond within the V_{H}, resulting in a significant energy improvement with no impact on CDR loop conformation. Together these analyses suggested that addition of this disulfide bond was likely to stabilize the MS2 antibody without negatively impacting antigen binding. A construct containing S49C and 169C V_{H} substitutions was made in the context of APE556 (see Table 1 below), and analyzed by DSC. This modification resulted in a 5.9° C. improvement in Fab Tₘ, such that the Fab and CH3 domains unfolded in a single melting transition, as shown in FIG. 4B. Biacore binding analysis confirmed complete retention of MS2 antigen binding affinity and revealed a 4-fold further improvement in K_{D} over APE556, as shown in Table 1.

TABLE I
Biophysical properties of stability-engineered anti-MS2 antibodies

| | V_{H} | V_{L} | C_{H}2 | Fab Tₘ | ΔFab Tₘ | C_{H}2 Tₘ | K_{D} |
|---|---|---|---|---|---|---|---|
| Antibody | mutations ^{a} | mutations ^{a} | mutations ^{a} | (° C.)^{b} | (° C.)^{c} | (° C.) | (nM) |
| scFv | WT | WT | | 66.8 | | n/a | 29 |
| APE443 | CDR graft into | CDR graft into | | 69.4 | 2.2 | 69.4 | 84 |
| | hV_{H}3-23 | hV_{K}2D-30 | | | | | |
| APE556 | — | F41Y, R51L, | | 77.8 | 11.0 | 69.4 | 27 |
| | | Y92F | | | | | |
| APE565 | S49C, I69C | F41Y, R51L, | | 83.7 | 16.9 | 67.6 | 7 |
| | | Y92F | | | | | |
| APE713 | — | — | L12C, K104C | 78.1 | 11.3 | 79.5 | n.d. |
| APE1032 | L5V, R19I, | P12A, T14L, | | 87.5 | 20.7 | 68.2 | n.d. |
| | S49C, I69C | F41Y, R51L, | | | | | |
| | | Y92F | | | | | |
| APE1025 | L5V, R19I, | M4L, P12A, | | 89.6 | 22.8 | 68.2 | 7 |
| | S49C, I69C | T14L, F41Y, | | | | | |
| | | R51L, Y92F | | | | | |
| APE979 | L5V, R19I, | M4L, P12A, | L12C, K104C | 90.3 | 23.5 | 84.5 | 2.5 |
| | S49C, I69C | T14L, F41Y, | | | | | |
| | | R51L, Y92F | | | | | |
| APE1051 | A23V | F41Y, R51L, | | n.d. | | n.d. | 15.1 |
| | | Y92F | | | | | |
| APE1052 | — | F41Y, R51L, | | n.d. | | n.d. | 4.2 |
| | | Y92F, Q27E, | | | | | |
| | | S27eT, H93R | | | | | |
| APE849 | F59S | F41Y, R51L, | | n.d. | | n.d. | 2.0 |
| | | Y92F, Q27E, | | | | | |
| | | S27eT, H93R | | | | | |
| APE830 | A23V | F41Y, R51L, | | n.d. | | n.d. | 1.6 |
| | | Y92F, Q27E, | | | | | |
| | | S27eT, H93R | | | | | |
| APE850 | A23V, F59S | F41Y, R51L, | | n.d. | | n.d. | 985 pM |
| | | Y92F, Q27E, | | | | | |
| | | S27eT, H93R | | | | | |
| APE1027 | L5V, R19I, | M4L, P12A, | L12C, K104C | 90.1 | 23.3 | 83.7 | 880 pM |
| | A23V, S49C, | T14L, QW27E, | | | | | |
| | F59S, I69C | S27eT, F41Y, | | | | | |
| | | R51L, Y92F, | | | | | |
| | | H93R | | | | | |

^{a} “X#Z” denotes that the amino acid residue(s) X at position # has (have) been replaced with amino acid residue(s) Z
^{b}Tₘ values determined by DSC
^{c}ΔFab Tₘ values are calculated relative to the scFv

### Disulfide Bond Engineering—Stabilization of the C_{H}2 Constant Domain

The IgG1 C_{H}2 is the least stable domain of the MS2 antibody, typically unfolding with a Tₘ in the 68-69° C. range. In order to improve C_{H}2 stability, and to assess the impact of C_{H}2 stabilization on the thermostability of neighboring domains, a disulfide bond was introduced into the α-MS2 C_{H}2. This disulfide bond incorporated L12C and K104C substitutions, which were previously shown to be stabilizing in the context of an isolated C_{H}2 domain (see Gong et al., J. Biol. Chem., 284: 14203-14210 (2009)). DSC analysis demonstrated an 8.7° C. increase in C_{H}2 Tₘ upon addition of this disulfide bond (compare Tₘ1, FIG. 4C). Stabilizing the C_{H}2 domain resulted in a 1.7° C. and 0.5° C. increase in Fab and C_{H}3 Tₘ, respectively.

### Computational Design

V_{H} and V_{L} sequences from the CDR-grafted anti-MS2 antibody were aligned to antibody structures in the RCSB Protein Databank (PDB) in order to identify high-resolution, homologous structures for use in a computational design process. Two structures were chosen for the V_{H}: PDB ID 3 KDM and 2VXS, each of which is 97% identical to the MS2 antibody V_{H}, excluding the CDRs. Three homologous light chain structures were similarly chosen: PDB ID 1T66, 1HPO, and 2H1P. These structures served as inputs for computational design.

The Rosetta suite of protein design software (Rosetta Design Group, LLC, University of Washington) was used as described (see Kuhlman et al., Proc. Natl. Acad. Sci. USA, 97: 10383-10388 (2000)) to further improve the stability of the anti-MS2 V_{H} and V_{L} domains using the homologous structures identified above. Potentially stabilizing amino acid substitutions were sampled in an iterative Metropolis Monte Carlo search, utilizing the backbone coordinates from each model structure and side-chain rotamer conformations taken from the Dunbrack backbone-dependent rotamer library, as described in Dunbrack et al., Protein Sci., 6: 1661-1681 (1997). CDR residues were excluded from the search in order to identify potentially stabilizing mutations more likely to be broadly applicable across multiple specificities, and to minimize impact on antigen binding. Native cysteine (C) residues crucial to variable domain stability were also excluded from the design. The remaining 65% of V_{H} and V_{L} framework residues were allowed to change to all amino acids except C.

Round 1 of each design allowed the remaining residues to change to all amino acids except C, searching a limited side chain conformational database containing rotameric models varied only around the first chi angle. A total of 100 independent runs were performed generating 100 sequences each. The second round limited the search only to those amino acids chosen during the first round. A larger rotamer library was used in this round that included chi-2 angle rotations. One hundred sequences were generated in each design round. Sequences that produced the lowest energy were analyzed, and the most frequently observed mutations giving the greatest energy improvement were chosen for testing in the context of the anti-MS2 antibody. On average, 52% of the residues subject to redesign were mutated from the wild type sequence, and these results were similar to those previously reported (see, e.g., Dantas et al., J. Mol. Biol., 332: 449-460 (2003); Korkegian et al., Science, 308: 857-860 (2005); and Kuhlman et al., supra).

Mutations identified by computational design were chosen for testing based on a combined criteria including design score, frequency of a particular mutation in multiple design runs, and mutations that promoted optimal packing within the hydrophobic core (see, e.g., Korkegian et al., supra). Site-directed mutagenesis was used to generate a total of ten heavy chain variants containing one or more amino acid substitutions. Two of the heavy chain mutations, L5V (+0.6° C.) and R19I (+0.3° C.), were found to be stabilizing when assessed by Thermofluor assay (ProteoStat Thermal Shift Stability Assay, Enzo Life Sciences, Farmingdale, N.Y.). Of the light chain mutations tested, the P12A, T14L double-mutant was the most stabilizing, giving a 1.5° C. increase in Fab Tₘ. The remaining mutations tested had either a neutral or negative impact on stability.

The four stabilizing mutations identified by computational design were combined into a single antibody construct, denoted APE1032, in the context of the aforementioned intra-V_{H} disulfide bond. These new mutations gave an additive 3.8° C. improvement in Fab Tₘ by DSC (see Table 1). This Tₘ increase was 1.4° C. greater than was expected based on previous analysis of the individual mutations.

### Consensus Design

An approach to consensus design for antibody stability is to compare a given variable region sequence to the consensus sequence for the most stable variable region family. While V_{H}3 is both the most common and the most stable of V_{H} domains, this is not true for Vκ2 (see, e.g., Knappik et al., J. Mol. Biol., 296, 57-86 (2000)). A comparison of Vκ2D-30 to the consensus sequence of the most stable V_{L} domain, Vκ3, identified residue M4 as different from the consensus L4. Additionally, mutation of residue 4 in the light chain from Met to Leu has been shown to be stabilizing in multiple antibody contexts (see, e.g., Benhar et al., J. Biol. Chem., 270: 23373-23380 (1995)). This residue is part of the hydrophobic core of the antibody where internal Met-to-Leu substitutions are known to improve hydrophobic core packing (see, e.g., Gassner et al., Proc. Natl. Acad. Sci. USA, 93: 12155-12158 (1996)). The M4L substitution was incorporated into the stabilized MS2 antibody, giving a further 2.1° C. improvement in Tₘ. The resulting antibody was denoted APE1025 (see Table 1).

This results of this example confirm the production of stable human antibody framework regions in accordance with the invention.

### EXAMPLE 3

This example demonstrates a method of producing stable human antibody framework regions using a combination of methods in accordance with the invention.

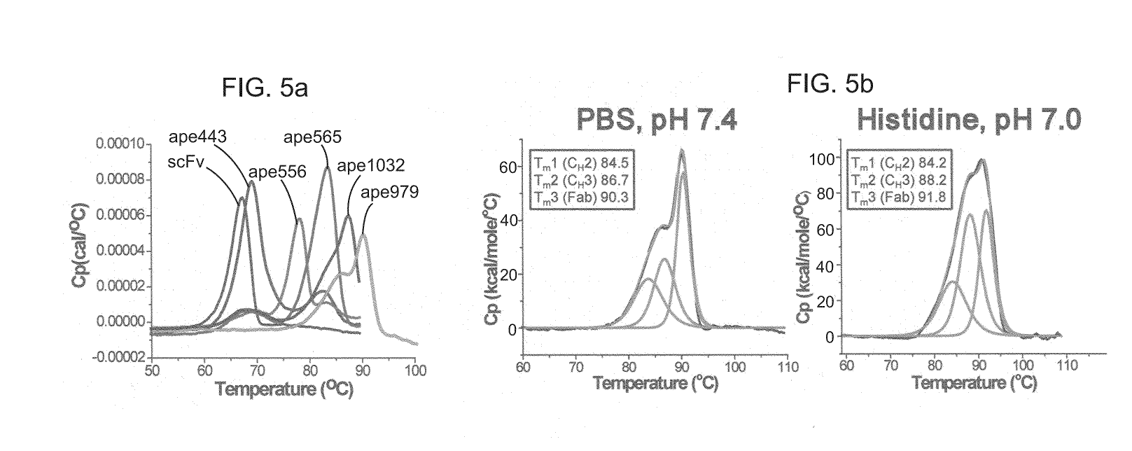

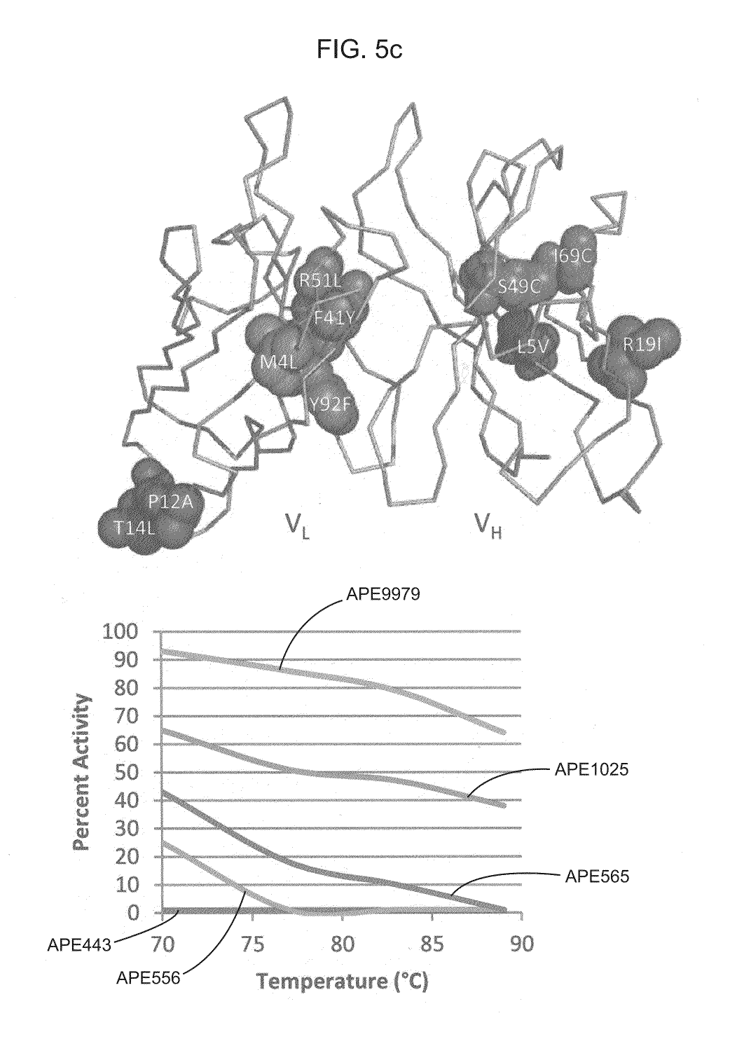

An anti-MS2 antibody fragment, denoted APE979, was generated to test the impact of combining the stabilizing amino acid changes described in Example 2 into a single antibody molecule. In this respect, a stabilized Fab domain was generating by using a combination of the methods described in Example 2, and the stabilized C_{H}2 domain described in Example 2 was introduced into the context of the stabilized Fab domain. This combination increased the Tₘ of the stabilized C_{H}2 to 84.5° C., which is a 15.1° C. improvement relative to the initial CDR-grafted antibody, as shown in FIG. 5B (as compared to FIG. 4C). The combined antibody additionally exhibited a 0.7° C. and 4.3° C. increase in Fab and C_{H}3 melting temperatures, respectively, upon incorporation of the stabilized C_{H}2 into APE1025. The total calorimetric heat change of unfolding (ΔH) for this antibody (ΔH=3.97×10⁵ kcal) was reduced by approximately 40% relative to the original CDR-grafted construct, APE443 (ΔH=6.65×10⁵ kcal). This is indicative of an increase in cooperativity of thermal unfolding for all three antibody domains. Final Tₘ improvements relative to the starting CDR-grafted antibody were 15.1° C. (C_{H}2), 20.9° C. (Fab), and 4.3° C. (C_{H}3), as shown in FIG. 5B. A non-exhaustive test of alternate buffer formulations identified a histidine-based buffer, pH 7.0, further improved the Fab Tₘ by nearly 2° C. to 92° C., as shown in FIG. 5B.

MS2-binding affinity of the APE979 Fab was assayed using a Biacore T200 (GE Healthcare, Waukesha, Wis.). For antibody fragments in scFv format, approximately 200 response units (RU) of MS2 were immobilized (CMS chip), and samples were tested in a concentration range from approximately 10-fold above the K_{D} to approximately 10-fold below the K_{D}. For full-length IgGs, a capture assay was used to allow accurate assessment of antibody affinity. Antibody was captured on a surface of approximately 3,000 RU mouse anti-human IgG Fc, and antigen was flowed over the captured IgG surface again using a range from 10-fold above to 10-fold below the K_{D} in each case. Surfaces were regenerated using 3 M MgCl₂. Association and dissociation kinetic values (kₐ and k_{d}) were determined from a best fit of the data with the 1:1 Langmuir global fitting procedure to sensorgrams using the Biacore T200 Evaluation Software, version 1.0. Biacore binding analysis confirmed that the APE979 antibody not only maintained full antigen binding activity, but exhibited a 30-fold improvement in MS2 affinity relative to the original CDR-grafted antibody, as shown in Table 1. Affinity improvement resulted from a 10-fold improvement in kₐ and a 3-fold improvement in k_{d}.

To further assess the extent of APE979 stabilization, a panel of progressively stabilized MS2 antibody variants was subjected to a one-hour thermal challenge at high temperature. Thermal challenge activity assays were performed by heating anti-MS2 antibody variants for one hour at a defined temperature (70-89° C.) and then cooling to 4° C. Heat-treated samples were compared to unheated samples for each antibody variant using Biacore analysis. Percent antigen-binding activity was determined by comparing experimental Rmax values for each sample to that of the unheated control. APE979 was the most stable antibody, maintaining over 60% activity after one hour at 89° C., as shown in FIG. 5C. In contrast, both the anti-MS2 scFv and the initial CDR-grafted antibody, APE443, showed a complete loss in activity after one hour at 70° C.

Because the conformation of the IgG Fc region, and particularly the lower hinge/C_{H}2, is important for Fc gamma receptor binding and the elicitation of antibody effector function, the binding of the combined stabilized antibody APE979, to the high affinity Fc gamma receptor, CD6450 was examined. Fc receptor binding was measured by immobilizing approximately 1,000 RU of soluble CD64 (R&D Systems, Minneapolis, Minn.) on a CM5 chip and testing antibody samples at 500 and 250 nM. Relative binding affinities were determined by comparing Rmax values between antibody samples. Stabilized APE979 exhibited no loss in CD64 binding in comparison to the starting antibody, APE443, and to a positive control antibody with known effector function.

The results of this example confirm that the protein stabilization method described herein can be used in combination to generate antibodies comprising stable framework regions in accordance with the invention.

### EXAMPLE 4

This example demonstrates a method of affinity maturing antibodies comprising stable framework regions.

A stable HEK293 c18 cell line expressing the anti-MS2 antibody APE556 (see Example 2) modified with a C-terminal transmembrane domain on the heavy chain for surface expression was generated as described in Bowers et al., Proc. Natl. Acad. Sci. USA, 108(51): 20455-20460 (2011). Antibody surface expression was confirmed by staining with FITC-labeled goat-anti-human C_{H}1. Cells were transiently transfected with an activation-induced cytidine deaminase (AID) expression vector for mutagenesis. After five days, cells were subjected to selection by fluorescence-activated cell sorting (FACS) using fluorescently labeled MS2 antigen. Co-expression of heavy and light chain genes with the AID enzyme induced SHM in the antibody resulting in in situ generation of genetic diversity in the antibody variable domain. Cells were stained by incubating for 30 minutes at 4° C. with MS2-DyLight-649 or MS2-WFP-DyLight-649 starting at 30 nM for early sorts and decreasing to 40 pM in the later sort rounds. To stain for IgG expression, FITC-Goat anti-Human IgG was added (1:2000) for 30 minutes at 4° C. The highest antigen binding cells, normalized for antibody expression, were sorted using a BD Influx cell sorter (BD Biosciences, San Jose, Calif.). Sequencing of 30 heavy chains (HC) and light chains (LC) from sorted cells subsequent to each FACS round revealed enriching SHM-induced mutations. High throughput sequencing was additionally utilized to generate over 100,000 heavy chain sequences for pre- and post-round 5 sort populations as described in Bowers et al., supra.

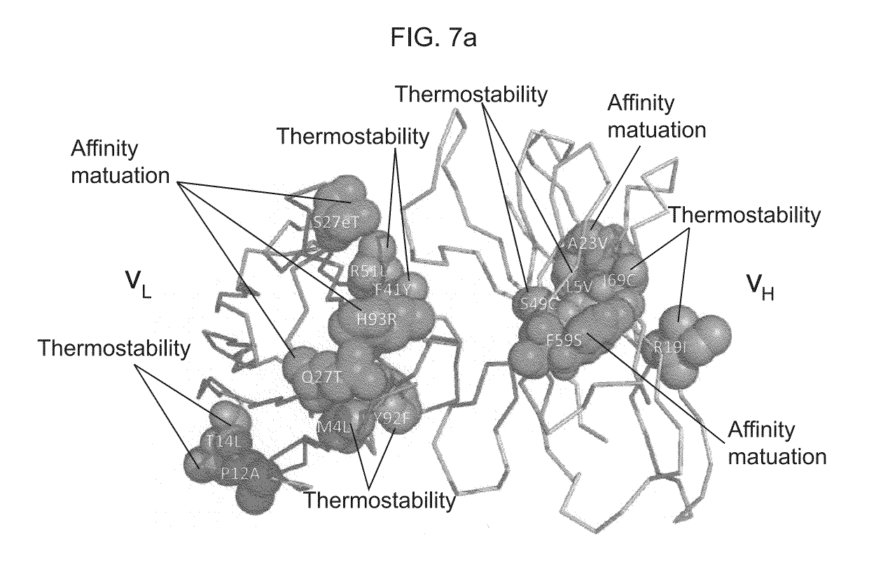

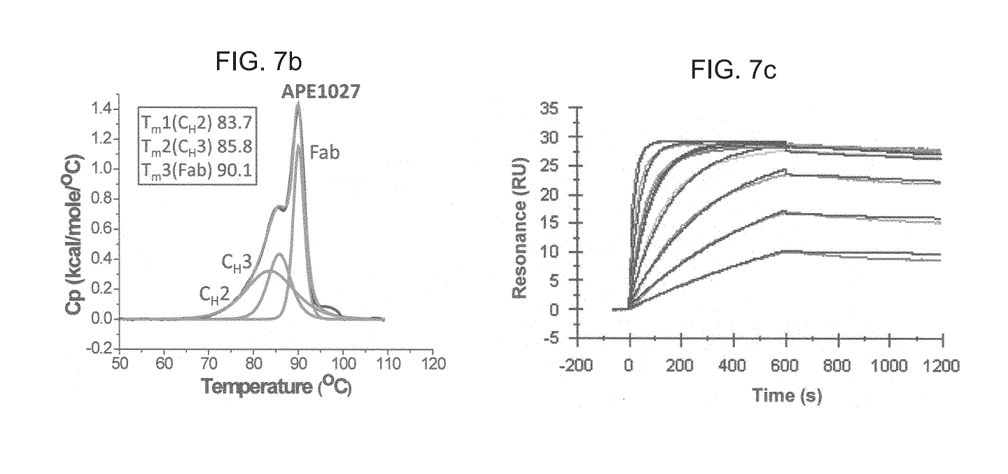

Two enriching mutations were observed in the heavy chain of APE556, i.e., A23V and F59S. The A23V substitution was located in the framework 1 region immediately adjacent to the first CDR loop, and F59S was located just outside of the CDR2, as shown in FIG. 7A.

In order to further explore SHM diversity in the light chain, four libraries of approximately 100 members each were constructed to recombine frequently observed SHM events. Corresponding amino acid substitutions were incorporated into the CDR loops of the CDR-grafted anti-MS2 antibody. For example, Library 1 consisted of the following variations: Q27QEL, S27eSTNGRDE, and H93HRLYNQ. Mutations were generated by overlap extension PCR using degenerate primers. Individual clones were sequenced, and HEK293 cultures were transiently transfected with HC/LC pairs in 96-well format. To rank variant antibodies based on K_{D}, supernatants were directly screened by Biacore 4000 using direct capture of secreted antibodies to measure 2×2. The best variants (K_{D}≦10 nM) were re-transfected on a larger scale, purified, and analyzed by Biacore T200 to obtain full binding kinetics data.

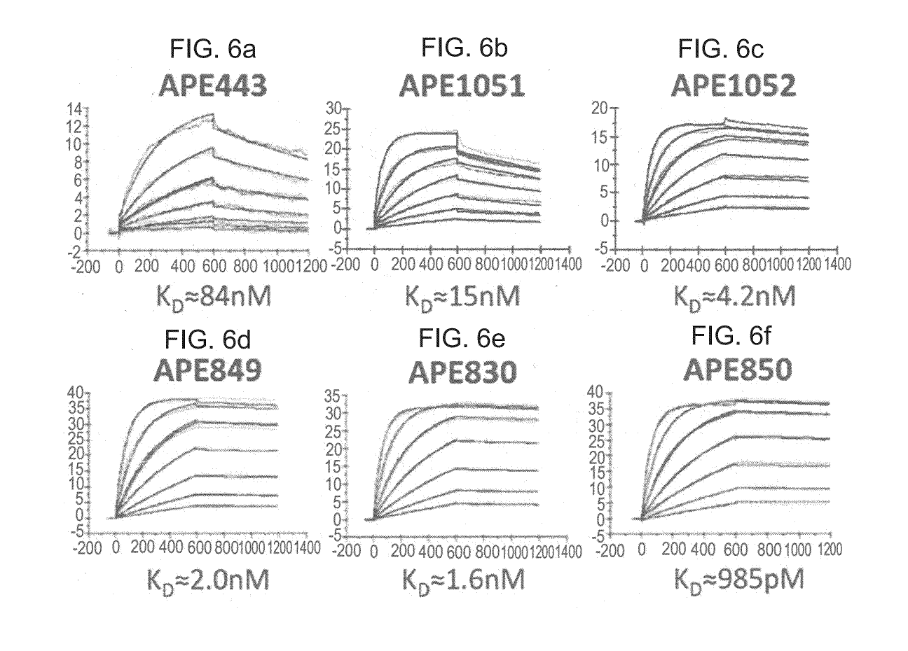

After mammalian cell expression, Biacore screening identified an antibody containing a triple mutant light chain with Q27E, S27eT, and H93R substitutions (APE1052) that gave a 20-fold improvement in K_{D} over the initial grafted antibody, as shown in FIG. 6A. The affinity of this antibody was improved 40-fold when combined with the F59S heavy chain mutation. Recombining all five mutations, reflected in FIGS. 6A-6E, into a single antibody, APE850, resulted in a final affinity for MS2 antigen of 985 pM, as shown in FIG. 6F. This represented an 85-fold improvement in K_{D} over the initial grafted antibody with a 12-fold improvement in kₐ and a 7-fold improvement in k_{d}.

In order to combine stability with increased binding affinity, CDR loops from the affinity matured APE85 anti-MS2 antibody were grafted onto the stabilized framework, with the resulting antibody denoted APE1027. Binding analysis by Biacore revealed a slight improvement in MS2 binding relative to the affinity matured antibody with a K_{D} of 880 pM, as shown in FIG. 7B. In addition, the grafted antibody completely maintained stability with a Fab Tₘ over 90° C., although there was a slight reduction (<1° C.) in C_{H}2 and C_{H}3 Tₘ, as shown in FIG. 7C.

This results of this example confirm the production of affinity matured antibodies comprising stable framework regions in accordance with the invention.

### EXAMPLE 5

This example demonstrates a method of producing a human antibody comprising stable framework regions using a combination of methods in accordance with the invention.

A mouse Fab targeting the Clostridium botulinum hemagglutanin 33 antigen (HA33) was selected for optimization as a part of DARPA Antibody Technology Program to develop stable, high-affinity antibodies for use in biosensors. The anti-HA33 Fab was generated from mice immunized with the HA33 antigen, and was obtained from the U.S. Army Edgewood Chemical Biological Center.

The anti-HA33 Fab was expressed transiently in HEK293 c-18 cells in chimeric IgG format with a human IgG1 Fc region. In this respect, heavy and light chain CDR1, CDR2, and CDR3 sequences derived from the mouse anti-HA33 antibody were grafted into the stable human hV_{H}3-23 and hVK2D-30 framework regions and formatted as a full-length immunoglobulin using human IgG1 kappa constant regions with an added intra-domain disulfide bond in the CH2 domain for stability. The starting mouse V_{H}14-3 framework region was 59% identical to the stable hV_{H}3-23 HC framework region, excluding the CDRs. Similarly, the starting mouse VLIgκV12-4 framework region was 57% identical to the stable human hVκ2D-30 framework region. A chimeric IgG comprising the mouse anti-HA33 mouse variable region with the same human constant regions, excluding the added disulfide, was generated for use as a control.

Full-length human IgG antibody variants also were expressed transiently in HEK293 c-18 cells, purified using a protein A/G agarose resin (Thermo Scientific, Waltham, Mass.), washed with 6 column-volumes of 1×PBS, pH 7.4, and eluted with 100 mM glycine, pH 3.0, followed by buffer exchange into 1×PBS, pH 7.4. All mutagenesis was carried out using the QuickChange II site-directed mutagenesis kit (Agilent Technologies, Santa Clara, Calif.).

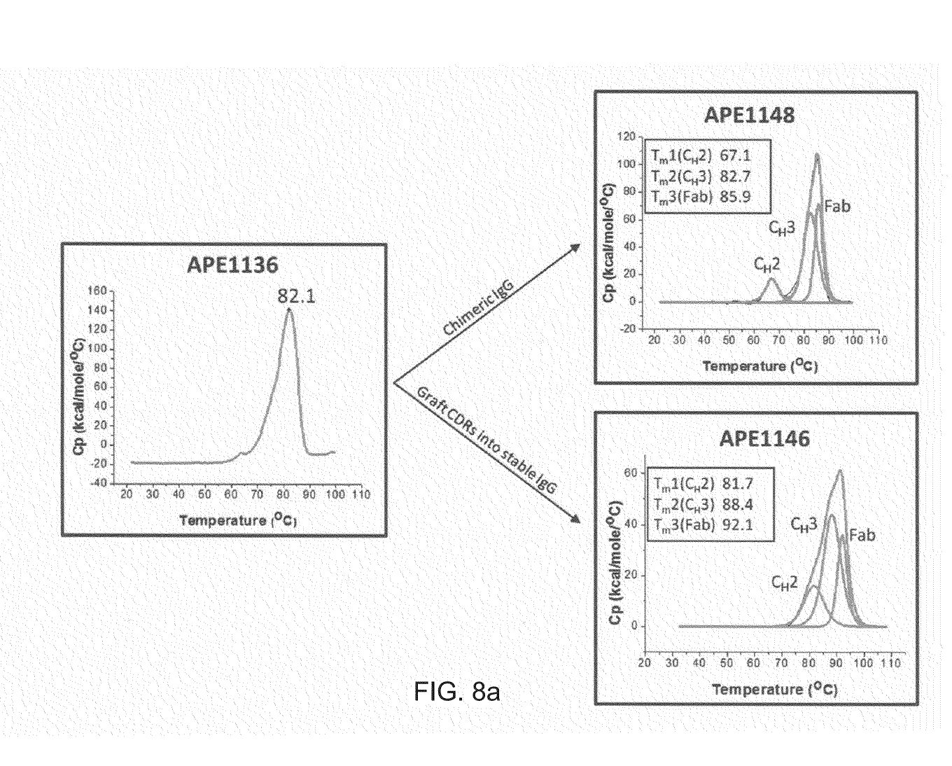

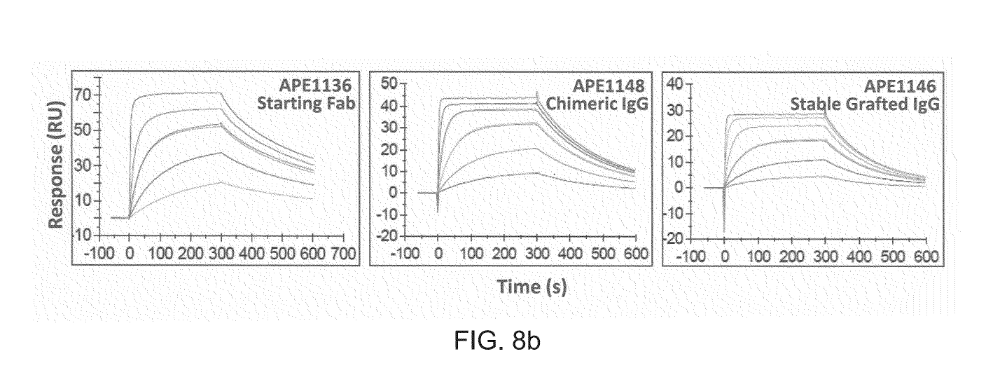

The thermal unfolding profiles of the anti-HA33 antibody variants were measured by differential scanning calorimetry (DSC) using the VP-Capillary DSC system (GE Healthcare, Waukesha, Wis.). All antibodies were tested in phosphate buffered saline (PBS), pH 7.4 at protein concentrations ranging from 0.7-1.0 mg/ml at a scan rate of 1° C./minute. Samples were heated from 20-110° C. Data analysis was performed using Origin 7 software. Transition mid-point values (Tₘ) were determined from the thermogram data using the non-two-state model which employs the Levenberg-Marquardt non-linear least-square method. DSC analysis of the stable grafted anti-HA33 antibody, denoted APE1146, exhibited a typical IgG curve with three unfolding transitions, representing the Fab, CH2, and CH3 domains of the antibody, as shown in FIG. 8A. A direct comparison of variable domains revealed a 10° C. improvement in transition mid-point values (Tₘ) relative to the starting anti-HA33 Fab (denoted APE1136), as shown in FIGS. 8A and 8B. A similar analysis of the chimeric Ig, denoted APE1148, revealed a 4° C. improvement in Tₘ relative to the starting anti-HA33 Fab APE1136, which indicates that some of the observed stabilization was the result of reformatting the Fab as a full-length human IgG.

Binding affinity measurements for the anti-HA33 antibody were obtained by surface immobilization of antigen. Specifically, kinetic analysis was performed using a Biacore T200 (GE Healthcare, Waukesha, Wis.). A capture assay was used to allow accurate assessment of antibody affinity and to minimize potential avidity effects due to the bivalent nature of the full-length antibody. Antibody (1 mg/ml) was captured on a surface of approximately 3,000 RU of mouse anti-human IgG Fc for 60 seconds at a flow rate of 10 ml/min, resulting in low capture levels between 50-100 RU. Antigen was flowed over the captured IgG surface again for 600 seconds at 30 mL/min using a range from 10-fold above to 10-fold below the K_{D} in each case. Surfaces were regenerated using 3 M MgCl₂. Association and dissociation kinetic values (kₐ and k_{d}) were determined from a best fit of the data with the 1:1 Langmuir global fitting procedure to sensorgrams using the Biacore T200 Evaluation Software, version 1.0. Kinetic analysis indicated that grafting HA33 CDRs into the stable human frameworks resulted in a modest 1.5-fold loss in HA33 antigen-binding affinity relative to the starting mouse Fab APE1136, with K_{D} values of 9 nM and 6 nM, respectively, as shown in Table 2 and FIG. 8B

Additionally, a subset of mutations known to improve stability in the context of the stable human framework regions was incorporated at analogous positions in the chimeric antibody denoted APE1196, as shown in Table 2. These mutations resulted in a further 4° C. increase in Tₘ, and the antibody retained wild-type HA33 binding affinity.

TABLE 2
Biophysical properties of anti-HA33 antibodies

| | V_{H} | V_{L} | Fab Tₘ | ΔFab Tₘ | | | |
|---|---|---|---|---|---|---|---|
| Antibody | mutations^{b} | mutations^{b} | (° C.)^{a} | (° C.) | K_{D} | | Source of Mutations |
| APE1136 | WT | WT | 82.1 | | 6 | nM | Starting antibody |
| APE1146 | CDR graft into | CDR graft into | 92.1 | 10.0 | 9 | nM | CDR graft into stable VH/VL |
| | stable hV_{H} | stable hV_{κ} | | | | | |
| APE1148 | WT | WT | 85.9 | 3.8 | 6 | nM | Chimeric starting antibody |
| APE1196 | WT with Q5V, | WT with M4L | 89.0 | 6.9 | 6 | nM | Stabilizing mutations incorporated |
| | G49C, I69C | | | | | | into chimeric antibody with |
| | | | | | | | stabilized C_{H}2 domain |
| APE1373 | — | G66E | 88.2 | 6.1 | 4 | nM | Affinity maturation |
| APE1481 | H35N | N50D, G66E | | | 910 | pM | Affinity maturation |
| APE1532 | H35N, Q64R | N50D, G66E | | | 660 | pM | Affinity maturation |
| APE1553 | H35N, A53L, | N50D, G66E | 88.2 | 6.1 | 30 | pM | Affinity maturation |
| | Q64R | | | | | | |
| APE1854 | H35N, A53L, | G66E | 92.0 | 9.9 | 45 | pM | Affinity maturation |
| | Q64R | | | | | | |

^{a}Tₘ values determined by DSC as described above
^{b}Mutations made in the context of the stable CDR-grafted antibody, APE1146, unless otherwise specified

The results of this example confirm the production of an antibody amino acid sequence comprising a stable human framework region in accordance with the invention.

### EXAMPLE 6

This example demonstrates a method of affinity maturing antibodies comprising stable framework regions.

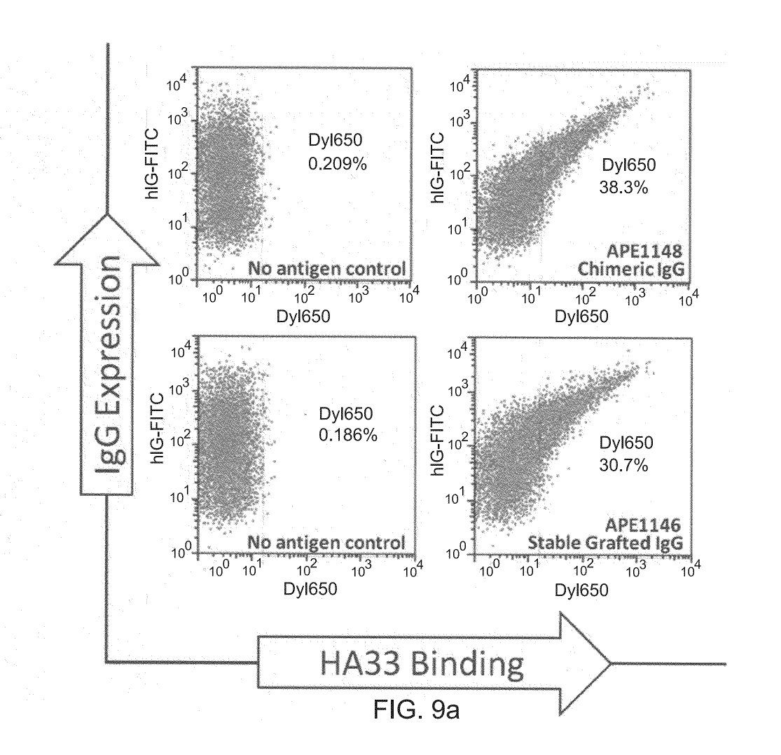

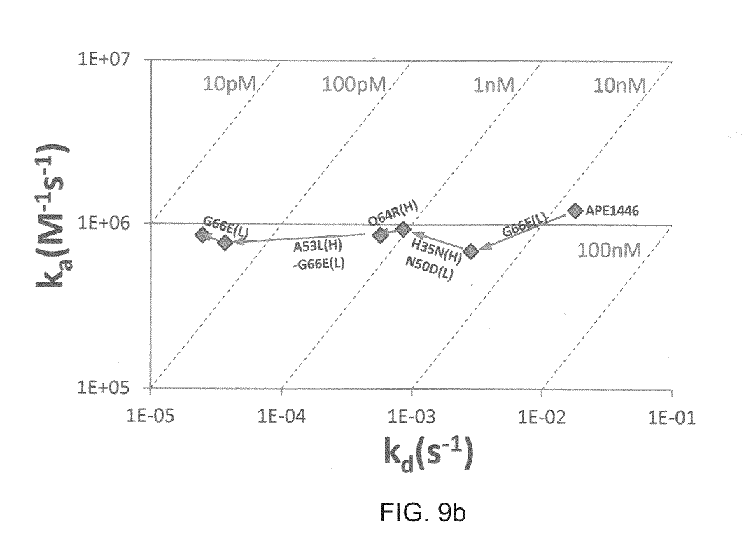

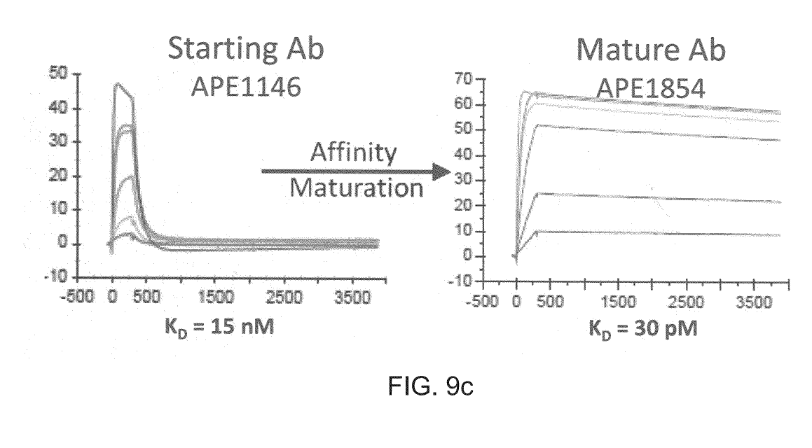

Stable HEK293 c18 cells expressing either the starting chimeric anti-HA33 antibody APE1148 (see Example 5) or the stable CDR-grafted antibody APE1146 (see Example 5), both of which were modified with a C-terminal transmembrane domain on the heavy chain for surface expression, were generated as described in Bowers et al., Proc. Natl. Acad. Sci. USA, 108(51): 20455-20460 (2011). Antibody surface expression was confirmed by staining with FITC-labeled goat-anti-human CH1. Cells were transiently transfected with an expression vector encoding activation-induced cytidine deaminase (AID) for mutagenesis. After five days, cells were subjected to selection by fluorescence-activated cell sorting (FACS) using fluorescently labeled HA33 antigen. Co-expression of heavy and light chain genes with the AID enzyme induced SHM in the antibody resulting in in situ generation of genetic diversity in the antibody variable domain. Cells were stained by incubating for 30 minutes at 4° C. with MS2-DyLight-649 or MS2-WFP-DyLight-649 starting at 30 nM for early sorts and decreasing to 40 pM in the later sort rounds. To stain for IgG expression, FITC-Goat anti-Human IgG was added (1:2000) for 30 minutes at 4° C. The highest antigen binding cells, normalized for antibody expression, were sorted using a BD Influx cell sorter (BD Biosciences, San Jose, Calif.). The results of the FACS analysis are show in FIG. 9A. Sequencing of 30 heavy chains (HC) and light chains (LC) from sorted cells subsequent to each FACS round revealed enriching SHM-induced mutations, which were incorporated into the stabilized antibody for kinetic analysis.

Three enriching mutations were observed in the chimeric APE1148 heavy chain, i.e., H35N, A53L, and Q64R. No light chain mutations were observed to enrich in the APE1148 light chain. In contrast, two enriching mutations were observed in the stable APE1146 light chain, i.e., N50D and G66E, though no mutations significantly enriched in the APE1146 heavy chain. Each of the APE1148 enriching heavy chain mutations and the APE1146 enriching light chain mutations was incorporated into the stable APE1146 framework, and each lead to improved HA33 binding affinity, with the most significant contribution provided by the A53L HC mutation, as shown in FIG. 9B. Additionally, the A53L substitution required the simultaneous enrichment of two mutations from the starting codon, GCG, to the Leucine encoding codon CTG. The affinity of the mature antibody containing all five enriching mutations, denoted APE1553, was 500-fold improved from the starting antibody, APE1146, with a KD of 30 pM, as shown in Table 2 and FIG. 9C.

In order to determine the impact of affinity maturation on the stability of the anti-HA33 antibody, APE1553 was analyzed by DSC as described above. DSC analysis revealed a 4° C. loss in Tₘ relative to APE1146. Removal of the G66E LC mutation was found to completely restore stability to this antibody with minimal impact on affinity, binding HA33 with a KD of 45 pM (see Table 2).

The results of this example confirm the production of affinity matured antibodies comprising stable framework regions in accordance with the invention.

### EXAMPLE 7

This example demonstrates a method of producing a human antibody comprising stable framework regions using a combination of methods in accordance with the invention.

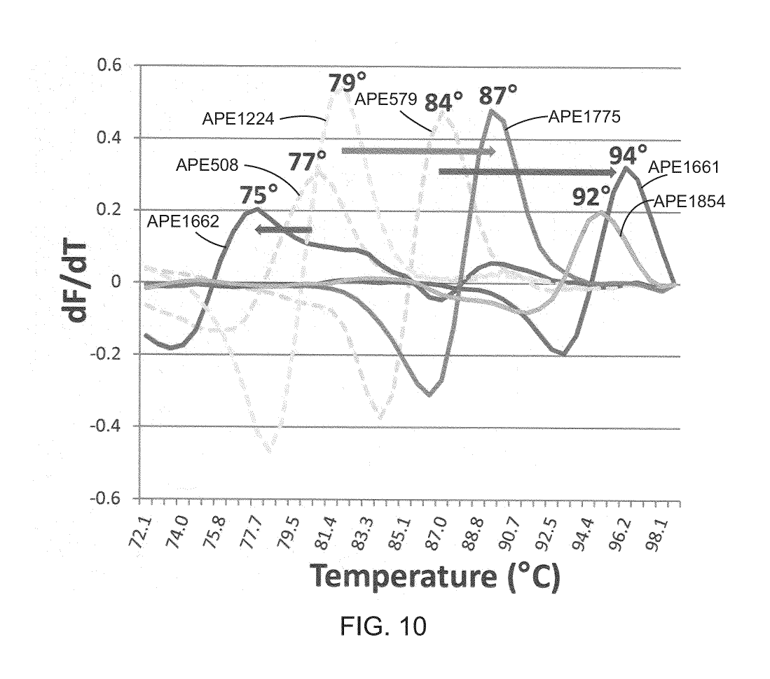

To further demonstrate the usefulness of the stable IgG framework described in the foregoing Examples, CDRs from the following therapeutically relevant antibodies were grafted onto the stable human hV_{H}3-23 IgG and hVκ2D-30 framework scaffolds: an anti-β-NGF antibody, an antibody targeting the C345C subunit of complement protein C5, an anti-IL-17A antibody, Denosumab, Omalizumab, Cetuximab, Trastuzumab, and an anti-TNFα antibody that was selected based on its significant divergence from the stable framework. The anti-β-NGF antibody was 94% identical to the stable HC framework and 61% identical to the stable LC framework, excluding the CDRs. The grafted anti-β-NGF antibody, APE1661, demonstrated a 10° C. improvement in melting temperature as measured by thermofluor assay, with a stabilized Tₘ of 94° C. The APE1661 antibody, however, exhibited a 2-fold loss in β-NGF binding affinity, as shown in Table 3. Similarly, the anti-C345C antibody was 94% identical to the stable HC framework and 70% identical to the stable LC framework. The grafted anti-C345C antibody exhibited an 8° C. improvement in Tₘ, though this antibody maintained full binding affinity for C345C, as shown in Table 3 and FIG. 10. APE508 binds to IL-17A with high affinity, and was the only antibody not stabilized by the graft, as shown in Table 3. APE508 is 89% identical to the stable HC framework and 70% identical to the stable LC framework. The grafted IL17-A antibody, APE1662, demonstrated a small 1.6-fold improvement in antigen binding affinity, as shown in Table 3 and FIG. 10.

TABLE 3
Biophysical properties of stable grafted antibodies

| Antibody | Antigen | Tₘ (° C.)^{a} | K_{D} | | Description |
|---|---|---|---|---|---|
| APE579 | β-NGF | 84 | 6 | nM | WT |
| APE1661 | β-NGF | 94 | 12 | nM | Stable Graft |
| APE508 | IL17-A | 77 | 340 | pM | WT |
| APE1662 | IL17-A | 75 | 210 | pM | Stable Graft |
| APE1224 | C5-C345C | 79 | 6 | nM | WT |
| APE1775 | C5-C345C | | 5 | nM | Stable Graft |
| APE1854 | HA33 | 92 | 45 | pM | Tₘ control |

^{a}Tₘ values determined by thermofluor assay as described above

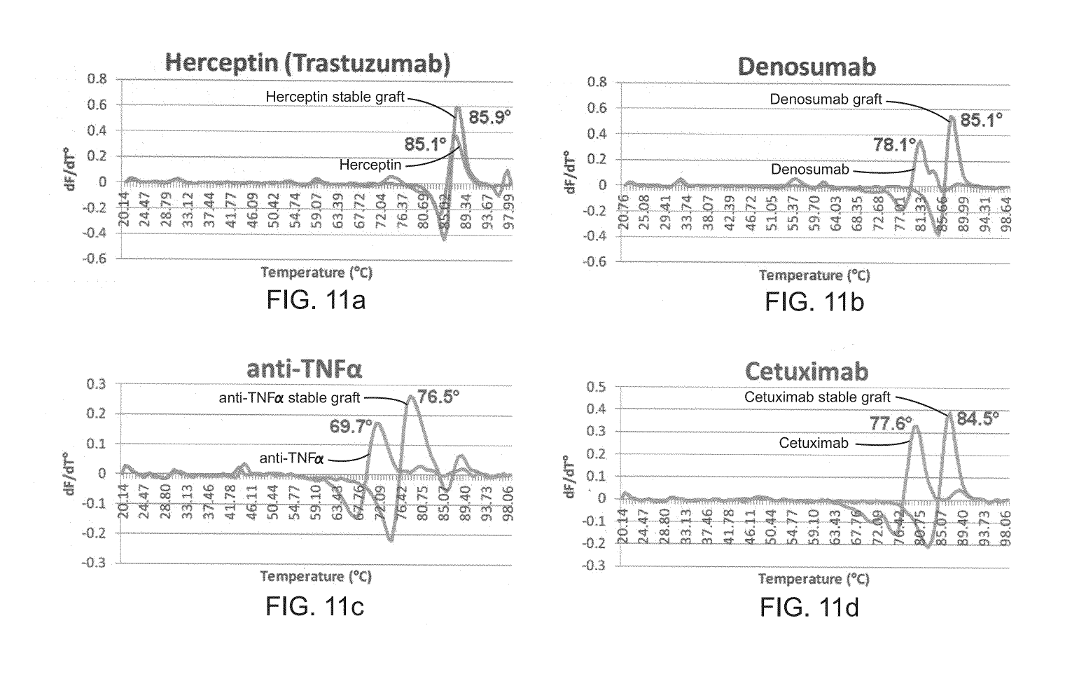

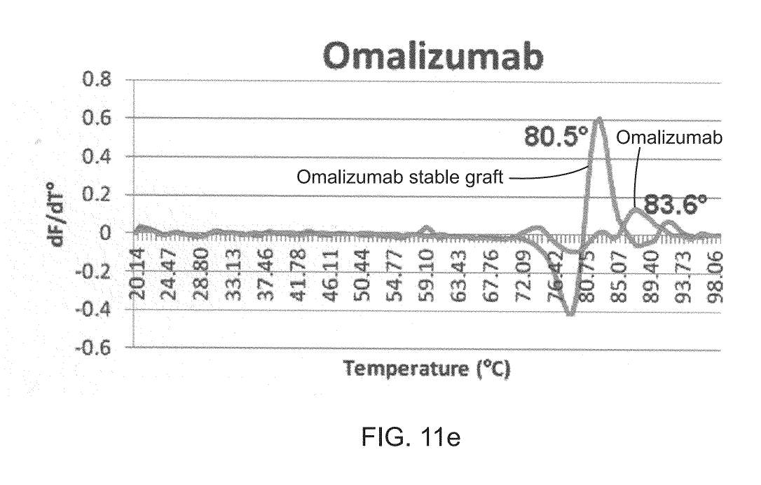

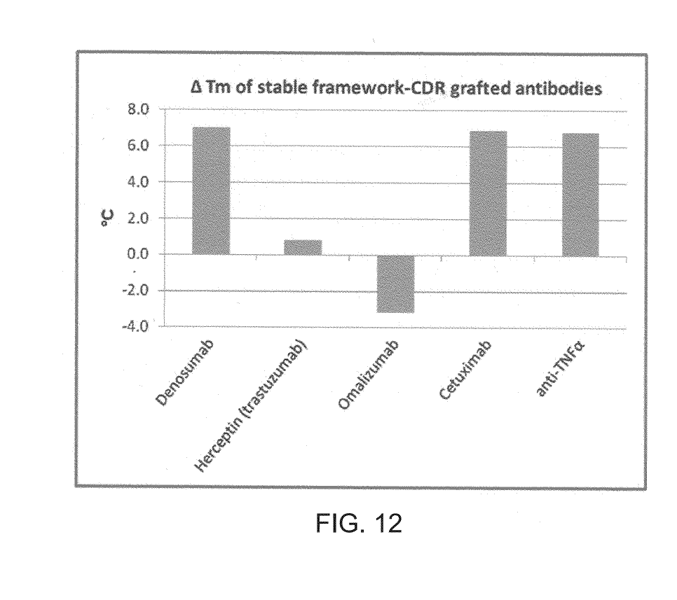

The results of the stabilization analysis of the other therapeutically relevant antibodies are set forth in Table 4 and FIGS. 11A-E. Both the original and stabilized variants of each antibody were tested for thermostability by thermofluor assay. Tₘ analysis was carried out using ProteoStat Thermal Shift Stability Assay (Enzo Life Sciences, Farmingdale, N.Y.) with samples at 0.1 mg/mL heated from 20-99° C. at a rate of 0.5° C./second. Peak fluorescence values indicated Tₘ values for each antibody. Substantial improvement in Tₘ was observed for Denosumab, Cetuximab, and anti-TNFα, with increases in Tₘ of 7° C., 6.9° C., and 6.8° C., respectively, as shown in FIG. 12. Trastuzumab exhibited a slight 0.8° C. improvement in Tₘ, while Omalizumab was not stabilized.

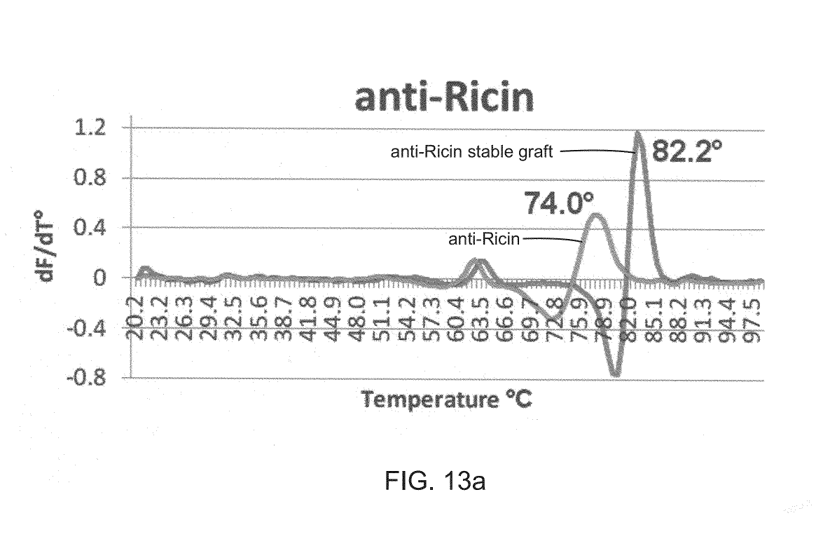

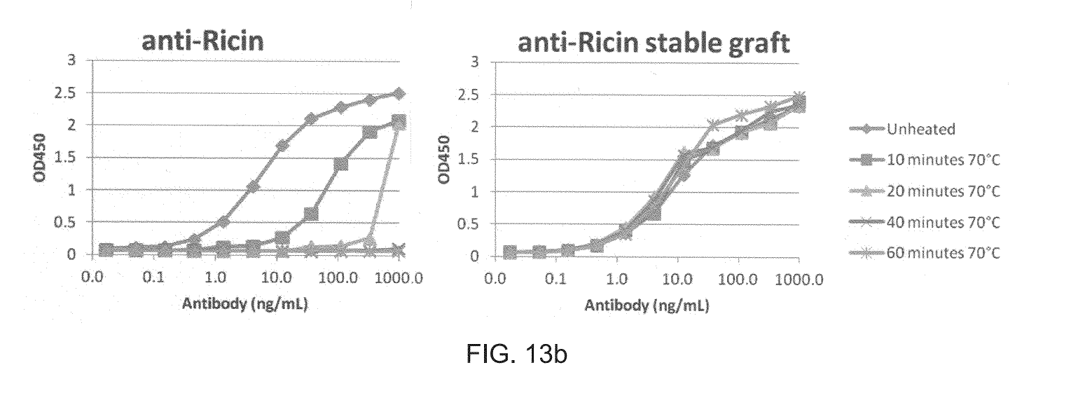

A mouse anti-Ricin antibody also was stabilized by grafting CDRs onto the stable IgG framework. The Tₘ of this antibody was improved by 8.2° C. by thermofluor assay, as shown in Table 4 and FIG. 13A. Anti-ricin antibody variants were heated at 70° C. for specific time periods at specific concentrations and then cooled to 4° C. before testing ricin binding activity by ELISA. For the ELISA assay, plates were coated with 1 μg/mL of antigen, blocked with 3% BSA in PBS, and incubated for 1 hour with each antibody variant before detection with goat anti-human IgG HRP. The stabilized antibody maintained full ricin binding activity after heating for 1 hour at 70° C., while the starting antibody lost all activity after heating for 40 minutes at 70° C., as shown in FIG. 13B.

TABLE 4

| | | | | | | | Stabilized |
|---|---|---|---|---|---|---|---|
| | | | CDR3 | # of SHM events | | | Amino Acid |
| | Starting VH | Starting VL | Lengths | in frameworks | Tₘ | Tₘ | SEQ ID NO |
| Antibody | framework | framework | (HC, LC) | (excluding CDRs) | Original | Stabilized | (HC, LC) |
| Denosumab | hIGHV3-23 | hIGKV3-20 | 13, 9 | 1 | 78.1 | 85.1 | 310, 319 |
| Omalizumab | hIGHV3-66 | hIGKV1-39 | 12, 9 | 5 | 83.6 | 80.5 | 308, 317 |
| Trastuzumab | hIGHV3-66 | hIGKV1-39 | 11, 9 | 5 | 85.1 | 85.9 | 311, 320 |
| anti-TNFα | mIGHV9-3-1 | mIGKV6-32 | 8, 9 | 9 | 69.7 | 76.5 | 307, 316 |
| Cetuximab | mIGHV2-2-3 | mIGKV5-48 | 11, 9 | 5 | 77.6 | 84.5 | 309, 318 |
| anti-βNGF | hIGHV3-23 | hIGKV1-27 | 14, 9 | 1 | 84.8 | 94.0 | 305, 314 |
| anti-IL17-A | hIGHV3-7 | hIGKV3-20 | 13, 9 | 5 | 77.5 | 74.3 | 306, 315 |
| anti-C5-C345C | hIGHV3-23 | hIGKV3-20 | 10, 10 | 1 | 79.5 | 87.4 | 304, 313 |
| anti-MS2 | mIGHV5-4 | mIGKV1-110 | 11, 9 | 6 | 74.4 | 92.4 | 322, 326 |
| anti-HA33 | mIGHV14-3 | mIGKV12-4 | 8, 9 | 3 | 82.1 | 92.0 | 324, 327 |
| anti-Ricin | mIGHV14-3 | mIGKV10-94 | 6, 9 | 2 | 74.0 | 82.2 | 312, 321 |

The results of this example confirm the stabilization of therapeutically relevant antibodies in accordance with the inventive method.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated amino acid sequence which comprises the framework regions of an immunoglobulin heavy chain variable region polypeptide of any one of SEQ ID NO: 1-SEQ ID NO: 189, except that each of two or more of residues 5, 19, 49, 50, 51, 64, 68, 69, 70, 71, 72, 73, and 75 thereof is replaced with a different amino acid residue.

2. The isolated amino acid sequence of claim 1, which comprises the framework regions of an immunoglobulin heavy chain variable region polypeptide of any one of SEQ ID NO: 1-SEQ ID NO: 189, wherein:
(a) residue 5 is replaced with a valine (V) residue,
(b) residue 19 is replaced with an isoleucine (I) residue,
(c) residue 49 is replaced with a cysteine (C) residue,
(d) residue 50 is replaced with a cysteine (C) residue,
(e) residue 51 is replaced with a cysteine (C) residue,
(f) residue 64 is replaced with a cysteine (C) residue,
(g) residue 68 is replaced with a cysteine (C) residue,
(h) residue 69 is replaced with a cysteine (C) residue, residue 70 is replaced with a cysteine (C) residue,
(j) residue 71 is replaced with a cysteine (C) residue,
(k) residue 72 is replaced with a cysteine (C) residue,
(l) residue 73 is replaced with a cysteine (C) residue,
(m) residue 75 is replaced with a cysteine (C) residue, or
(n) any combination of two or more of (a) through (m).

3. The isolated amino acid sequence of claim 1, which comprises the framework regions of an immunoglobulin heavy chain variable region polypeptide of any one of SEQ ID NO: 1-SEQ ID NO: 189, wherein:
(a) residue 5 is replaced with a valine (V) residue,
(b) residue 19 is replaced with an isoleucine (I) residue,
(c) residue 49 is replaced with a cysteine (C) residue, and
(d) residue 69 is replaced with a cysteine (C) residue.

4. An isolated amino acid sequence which comprises the framework regions of an immunoglobulin light chain variable region polypeptide of any one of SEQ ID NO: 190-SEQ ID NO: 291, except that each of two or more of residues 4, 12, and 14 thereof is replaced with a different amino acid residue.

5. The isolated amino acid sequence of claim 4, which comprises the framework regions of an immunoglobulin light chain variable region polypeptide of any one of SEQ ID NO: 190-SEQ ID NO: 291, wherein:
(a) residue 4 is replaced with a leucine (L) residue,
(b) residue 12 is replaced with an alanine (A) residue,
(c) residue 14 is replaced with a leucine (L) residue, or
(d) any combination of two or more of (a) through (c).

6. The isolated amino acid sequence of claim 4, which comprises the framework regions of an immunoglobulin light chain variable region polypeptide of any one of SEQ ID NO: 190-SEQ ID NO: 291, wherein:
(a) residue 4 is replaced with a leucine (L) residue,
(b) residue 12 is replaced with an alanine (A) residue, and
(c) residue 14 is replaced with a leucine (L) residue.

7. An isolated amino acid sequence comprising the constant region of an immunoglobulin heavy chain polypeptide comprising of any one of SEQ ID NO: 292-SEQ ID NO: 295, except that each of residues 12 and 104 thereof is replaced with a different amino acid residue.

8. The isolated amino acid sequence of claim 7, which comprises the constant region of an immunoglobulin heavy chain polypeptide comprising any one of SEQ ID NO: 292-SEQ ID NO: 295, wherein:
(a) residue 12 is replaced with a cysteine (C) residue, and
(b) residue 104 is replaced with a cysteine (C) residue.

9. The isolated amino acid sequence of claim 1, which comprises a transition mid-point value (Tₘ) in vitro of 70-100° C.

10. An isolated antigen binding agent comprising the amino acid sequence of claim 1.

11. The isolated antigen binding agent of claim 10, which is antibody, an antibody conjugate, or an antigen-binding fragment thereof.

12. The isolated antigen binding agent of claim 10, which is an antibody fragment selected from the group consisting of F(ab′)2, Fab′, Fab, Fv, scFv, dsFv, dAb, and a single chain binding polypeptide.

13. An isolated or purified nucleic acid sequence encoding the amino acid sequence of claim 1.

14. A vector comprising the isolated or purified nucleic acid molecule of claim 13.

15. An isolated cell comprising the vector of claim 14.

16. A composition comprising the isolated amino acid sequence of claim 1 and a pharmaceutically acceptable carrier.

17. A composition comprising the vector of claim 14 and a pharmaceutically acceptable carrier.

18. A method of improving the antigen-binding activity of the amino acid sequence of claim 1, which method comprises subjecting a nucleic acid sequence encoding the amino acid sequence to somatic hypermutation (SHM), whereby the antigen-binding activity of the amino acid sequence is improved.

19. A method of improving the antigen-binding activity of the amino acid sequence of claim 1, which method comprises deleting 1-10 amino acid residues from the amino acid sequence, whereby the antigen-binding activity of the amino acid sequence is improved.

20. The method of claim 18, wherein the antigen-binding activity is measured as antigen binding affinity, antigen binding specificity, and/or antigen cross-reactivity.

21. A method of producing the isolated amino acid sequence of claim 1, which method comprises providing an amino acid sequence which comprises an unmodified framework region of an immunoglobulin heavy chain variable region, and subjecting the amino acid sequence to one or more of the following:
(a) grafting one or more non-native complementarity determining regions (CDR) into the amino acid sequence,
(b) introducing one or more non-native disulfide bonds into the amino acid sequence,
(c) introducing one or more non-native consensus amino acid residues into the amino acid sequence, or
(d) introducing one or more stabilizing amino acid residues into the amino acid sequence, whereby a thermostable framework region of an immunoglobulin heavy chain variable region is produced.

22. The method of claim 21, which further comprises subjecting a nucleic acid sequence encoding the thermostable framework region of an immunoglobulin heavy chain variable region to somatic hypermutation.

23. A method of preparing the isolated amino acid sequence of claim 4, which method comprises providing an amino acid sequence which comprises an unmodified framework region of an immunoglobulin light chain variable region, and subjecting the amino acid sequence to one or more of the following:
(a) grafting one or more non-native complementarity determining regions (CDR) into the amino acid sequence,
(b) introducing one or more non-native disulfide bonds into the amino acid sequence,
(c) introducing one or more non-native consensus amino acid residues into the amino acid sequence, or
(d) introducing one or more stabilizing amino acid residues into the amino acid sequence, whereby a thermostable framework region of an immunoglobulin light chain variable region is produced.

24. The method of claim 23, which further comprises subjecting a nucleic acid sequence encoding the thermostable framework region of an immunoglobulin light chain variable region to somatic hypermutation.

25. An isolated antigen binding agent comprising the amino acid sequence of claim 4.

26. An isolated antigen binding agent comprising the amino acid sequence of claim 7.

27. An isolated or purified nucleic acid sequence encoding the amino acid sequence of claim 4.

28. A vector comprising the isolated or purified nucleic acid molecule of claim 27.

29. An isolated cell comprising the vector of claim 28.

30. An isolated or purified nucleic acid sequence encoding the amino acid sequence of claim 7.

31. A vector comprising the isolated or purified nucleic acid molecule of claim 30.

32. An isolated cell comprising the vector of claim 31.

33. A composition comprising the isolated amino acid sequence of claim 4 and a pharmaceutically acceptable carrier.

34. A composition comprising the isolated amino acid sequence of claim 7 and a pharmaceutically acceptable carrier.

35. A composition comprising the antigen binding agent of claim 10 and a pharmaceutically acceptable carrier.

36. A composition comprising the antigen binding agent of claim 25 and a pharmaceutically acceptable carrier.

37. A composition comprising the antigen binding agent of claim 26 and a pharmaceutically acceptable carrier.
